(19) United States
(12) Patent Application Publication
(10) Pub. No.: US 2024/0392344 A1
(43) Pub. Date: Nov. 28, 2024

(54) DEVICES AND METHODS FOR TARGETED POLYNUCLEOTIDE APPLICATIONS

(71) Applicant: DIMENSION GENOMICS INC, LA JOLLA, CA (US)

(72) Inventors: Michael David Austin, San Diego, CA (US); William K. Ridgeway, Encinitas, CA (US)

(73) Assignee: DIMENSION GENOMICS INC, LA JOLLA, CA (US)

(21) Appl. No.: 18/694,652

(22) PCT Filed: Sep. 28, 2022
(86) PCT No.: PCT/US22/44998

§ 371 Date: Mar. 22, 2024

Related U.S. Application Data
(60) Provisional application No. 63/250,119, filed on Sep. 29, 2021.

(51) Int. Cl. C12Q 1/6809; C12Q 1/6876

(52) U.S. Cl. CPC C12Q 1/6809; C12Q 1/6876; C12Q 2600/154

(57) ABSTRACT

Disclose herein are methods and devices for interrogating a region of interest within an immobilized nucleic acid molecule with a contact probe, where said region of interest is determined at least in part through an analysis of said molecule's physical map generated by optical interrogation.

### CROSS-REFERENCE TO RELATED APPLICATIONS

This document claims the benefit of priority to U.S. Provisional Application Ser. No. 63/250,119, filed Sep. 29, 2021, the contents of which are hereby incorporated by reference.

### BACKGROUND

Optical physical mapping methods of long nucleic acid molecules have demonstrated an effective means of generating a high-throughput genomic maps with a universal labelling scheme that does not require significant a priori knowledge of the underlying sequence of the molecules. These physical maps are extremely powerful, as they can provide contextual information as to where various genomic information and higher order structures are physically located with respect to each other within the molecule via analysis of an alignment of the physical map to a reference. This information is not directly available from high throughput shotgun sequencing, and computational approaches to assemble genomes are complex and ultimately limited in their ability to make inferences. The optical physical map's resolution however is limited by many factors, most significantly the optical system used for imaging, and so the biological and clinical utility of the data generated with such an optical physical map may be insufficient for certain applications.

Interrogating a long nucleic acid molecule with a contact probe, such as an AFM, offers the opportunity to analyze a long nucleic acid molecule at much higher resolution, including potentially single nucleotide resolution. However, such resolution comes at the cost of extremely low throughput interrogation, limiting the applications to only very targeted interrogations of a small fraction of a human genome. Conversely, optical interrogation is capable of extremely high-throughput interrogation, demonstrating the ability to interrogate several whole human genomes a day at significant coverage. There exists a need to couple these methods with a physical map such that an analysis of the physical map generated by optical interrogation provides not only a region of interest (ROI) within the genome to examine, but also the physical coordinates in xyz space in which said ROI is located, such that a contact probe can be guided to that ROI to perform a subsequent high resolution interrogation. With such a method, ROIs can be selected at least partially based on the positional relationship of various genomic information within the molecule, and in many cases without a priori knowledge of the precise ROI sequence composition.

### SUMMARY OF THE INVENTION

Disclosed here are methods and devices for interrogating with a contact probe at least one region of interest (ROI) within at least one long nucleic acid molecule from a sample. The methods generally involve a modified and at least partially immobilized at least one nucleic acid on a substrate or open fluidic device in a substantially elongated configuration, where the degree of modification along or within the at least one molecule comprises at least two bound labeling bodies that generate a physical map along or within the at least one molecule, and whose pattern can be optically interrogated and analyzed at least in part by an alignment of said physical map to a reference to identify specific ROI(s) within the physical map(s) of the at least one molecule, along with the ROI(s)'s corresponding physical coordinates with respect to the underlying substrate or open fluidic device; and then further interrogating the ROI(s) by directing a contact probe to interrogate within the desired coordinates of the ROI(s). The present invention further provides a computer program and interrogation system product for use in a subject method.

Also disclosed are methods and devices for interrogating with a contact probe at least one region of interest (ROI) within at least one long nucleic acid molecule from a sample. The methods generally involve a at least partially immobilized at least one nucleic acid on a substrate or open fluidic device in a substantially elongated configuration, where the higher order nucleic acid structure(s) along or within the at least one molecule comprises a physical map along or within the at least one molecule, and whose pattern can be optically interrogated and analyzed at least in part by an alignment of said physical map to a reference to identify specific ROI(s) within the physical map(s) of the at least one molecule, along with the ROI(s)'s corresponding physical coordinates with respect to the underlying substrate or open fluidic device; and then further interrogating the ROI(s) by directing a contact probe to interrogate within the desired coordinates of the ROI(s). The present invention further provides a computer program and interrogation system product for use in a subject method.

Disclosed herein are methods of characterizing a region of interest of a nucleic acid molecule. Aspects of this embodiment variously comprise one or more of the following elements: attaching the nucleic acid molecule to a surface of at least one point on the nucleic acid; determining a physical map of at least a portion of the nucleic acid molecule; comparing the physical map of at least a portion of the nucleic acid molecule to a Reference to identify a segment of the physical map that has a co-relationship to the at least a segment of the Reference; correlating the segment of the physical map of at least a portion of the nucleic acid molecule that differs from the correlating Reference to a region of interest on the nucleic acid molecule; subjecting the region of interest on the nucleic acid molecule to a second physical characterization. In some aspects, the elements are performed in order as recited above. In some aspects, the elements are performed on a single nucleic acid molecule.

In some aspects the surface is exposed. In some aspects the surface is not interior to a flow cell. In some aspects the surface is not interior to a fluidic device. In some aspects the surface is accessible to exterior mechanical manipulation. In some aspects attaching the nucleic acid molecule comprises binding a chromatin constituent associated with the nucleic acid molecule to a chromatin constituent affinity partner. In some aspects attaching comprises immobilizing the nucleic acid to the surface. In some aspects determining a physical map of at least a portion of the nucleic acid molecule comprises determining an AT concentration of the at least a portion of the nucleic acid molecule. In some aspects determining a physical map of at least a portion of the nucleic acid molecule comprises determining a GC concentration of the at least a portion of the nucleic acid molecule. In some aspects determining a physical map of at least a portion of the nucleic acid molecule comprises determining a nucleic acid subsequence pattern for a recurring subsequence of the at least a portion of the nucleic acid molecule. In some aspects the nucleic acid subsequence pattern comprises a repeat element pattern. In some aspects the repeat element comprises a transposon. In some aspects the repeat element comprises a retroelement. In some aspects the repeat element comprises an Alu repeat. In some aspects the repeat element comprises an octomer. In some aspects the repeat element comprises a hexamer. In some aspects determining a physical map of at least a portion of the nucleic acid molecule comprises determining a nucleic acid higher order structure pattern. In some aspects the nucleic acid higher order structure pattern comprises a nucleic acid knot pattern. In some aspects the nucleic acid higher order structure pattern comprises a nucleic acid binding protein binding pattern. In some aspects the nucleic acid higher order structure pattern comprises a topological pattern. In some aspects determining a physical map of at least a portion of the nucleic acid molecule comprises determining a nucleic acid associate protein binding pattern. In some aspects the nucleic acid associate protein binding pattern is a chromatin protein binding pattern. In some aspects the nucleic acid associate protein binding pattern is an exogenous protein binding pattern. In some aspects the nucleic acid associate protein binding pattern is a CRISPR protein complex binding pattern. In some aspects the nucleic acid associate protein binding pattern is a transcription factor binding pattern. In some aspects the nucleic acid associate protein binding pattern is a histone binding pattern. he method of any of the previous embodiments, wherein the nucleic acid associate protein binding pattern is a modified histone binding pattern. In some aspects determining a physical map of at least a portion of the nucleic acid molecule comprises determining a nucleic acid modification pattern. In some aspects the nucleic acid modification pattern results from contacting bound labelling bodies. In some aspects the nucleic acid modification pattern is a DNA methylation pattern. In some aspects determining a physical map of at least a portion of the nucleic acid molecule does not comprise sequencing the at least a portion of the nucleic acid molecule. In some aspects determining a physical map of at least a portion of the nucleic acid molecule requires no more than 1 second. In some aspects determining a physical map of at least a portion of the nucleic acid molecule requires no more than 1/100 of a second. In some aspects the comparing comprises aligning. In some aspects aligning the physical map of at least a portion of the nucleic acid molecule to a reference comprises identifying a segment of the physical map of at least a portion of the nucleic acid molecule that is absent from the reference. In some aspects aligning the physical map of at least a portion of the nucleic acid molecule to a Reference comprises identifying a segment of the physical map of at least a portion of the nucleic acid molecule that is inverted relative to the Reference. In some aspects aligning the physical map of at least a portion of the nucleic acid molecule to a Reference comprises identifying a segment of the physical map of at least a portion of the nucleic acid molecule is translocated relative to the Reference. In some aspects aligning the physical map of at least a portion of the nucleic acid molecule to a Reference comprises identifying a segment of the physical map of at least a portion of the nucleic acid molecule that that is duplicated relative to the Reference. In some aspects aligning the physical map of at least a portion of the nucleic acid molecule to a Reference comprises identifying a segment of the physical map of at least a portion of the nucleic acid molecule that differs by at least 5% relative to the Reference. In some aspects aligning the physical map of at least a portion of the nucleic acid molecule to a Reference comprises identifying a segment of the physical map of at least a portion of the nucleic acid molecule that that differs by at least 10% relative to the Reference. In some aspects aligning the physical map of at least a portion of the nucleic acid molecule to a Reference comprises identifying a segment of the physical map of at least a portion of the nucleic acid molecule that differs by at least 20% relative to the Reference. In some aspects aligning the physical map of at least a portion of the nucleic acid molecule to a Reference to identify a segment of the physical map of at least a portion of the nucleic acid molecule that differs by at least 50% relative to the Reference. In some aspects the Reference comprises a predictive physical map. In some aspects the Reference is derived from a nucleic acid sequence. In some aspects the nucleic acid sequence is a genomic sequence. In some aspects the nucleic acid sequence is derived from a reference organism. In some aspects the nucleic acid sequence is derived from a cancer-free cell. In some aspects the Reference is previously obtained. In some aspects the Reference is concurrently obtained. In some aspects the Reference is obtained from a tissue distal to a tissue from which the nucleic acid molecule is obtained.

In some aspects the tissue and the nucleic acid are obtained from a common individual. In some aspects the tissue is disease free. In some aspects the tissue is cancer free. In some aspects the nucleic acid molecule is obtained from a cancerous cell. In some aspects the tissue is cancerous. In some aspects the tissue exhibits a disease. In some aspects the nucleic acid molecule is obtained from a healthy cell. In some aspects the nucleic acid molecule is obtained from a disease-free cell. In some aspects the tissue and the nucleic acid differ in age. In some aspects the tissue is a preserved tissue. In some aspects the nucleic acid is from a later obtained cell. In some aspects the nucleic acid is from an earlier obtained cell. In some aspects correlating the segment of the physical map of at least a portion of the nucleic acid molecule that differs from the Reference to a region of interest on the nucleic acid molecule comprises identifying a location of the region of interest on the nucleic acid molecule on the surface. In some aspects subjecting the region of interest on the nucleic acid molecule to a second physical characterization comprises removing a cover slip covering the nucleic acid molecule. In some aspects subjecting the region of interest on the nucleic acid molecule to a second physical characterization occurs on an exposed area of the surface. In some aspects subjecting the region of interest on the nucleic acid molecule to a second physical characterization comprises generating a second physical characterization of the region of interest on the nucleic acid molecule. In some aspects the second physical characterization depicts a characteristic different from that initially characterized. In some aspects the second physical characterization depicts an AT pattern. In some aspects the second physical characterization depicts a GC pattern. In some aspects the second physical characterization depicts a protein binding pattern. In some aspects the second physical characterization depicts secondary structure concentration. In some aspects the second physical characterization depicts a histone modification pattern. In some aspects the second physical characterization depicts a nucleic acid modification pattern. In some aspects the second physical characterization depicts an octomer distribution pattern. In some aspects the second physical characterization depicts a hexamer distribution pattern. In some aspects the second physical characterization depicts a transposable element pattern. In some aspects the second physical characterization comprises a nucleic acid probe binding pattern. In some aspects the second physical characterization presents the number of repeats of a repeated element. In some aspects the nucleic acid probe binding pattern is assayed using a fluorophore bound to a nucleic acid probe. In some aspects the nucleic acid probe binding pattern is assayed using a barcode tag bound to a nucleic acid probe. In some aspects the second physical characterization comprises obtaining a nucleic acid sequence. In some aspects the second physical characterization comprises subjecting the region to a contact probe. In some aspects the contact probe determines a nucleic acid sequence for at least a portion of the region. In some aspects the contact probe is an atomic force microscopy probe. In some aspects the contact probe determines a position of the region in an axis perpendicular to the region. In some aspects the second physical characterization comprises physically manipulating the region.

In some aspects the portion of the nucleic acid that differs from the reference is inverted relative to the reference. In some aspects the portion of the nucleic acid that differs from the reference is translocated relative to the reference. In some aspects the portion of the nucleic acid that differs from the reference is duplicated relative to the reference. In some aspects the portion of the nucleic acid that differs from the reference is absent from the reference. In some aspects the second physical map comprises a sequence of the portion of the nucleic acid that differs from the reference. In some aspects the sequence is determined in situ. In some aspects the sequence is determined by direct manipulation of the nucleic acid on the surface. In some aspects the sequence is determined using atomic force microscopy. In some aspects the sequence is determined using hybridization to a probe of known sequence. In some aspects the nucleic acid is fixed to a surface. In some aspects the surface is exposed. In some aspects the surface is not a flow cell interior. In some aspects the surface is accessible to physical manipulation. In some aspects the surface is covered by a removable cover slip.

In some aspects the physical map differs from the reference. In some aspects the landmark is a known variable region on the reference. In some aspects the landmark aligns with the region of interest. In some aspects the landmark is removed a known distance from a region on the reference that corresponds to the region of interest on the nucleic acid molecule. In some aspects the second physical characterization comprises a higher resolution map at the region of interest on the nucleic acid molecule than the physical map. In some aspects the second physical characterization comprises a nucleic acid sequence of the region of interest of the nucleic acid. In some aspects the second physical characterization comprises determining a second physical map of the region of interest. In some aspects determining the physical map on the nucleic acid molecule does not preclude subjecting the region of interest on the nucleic acid molecule to a second physical characterization. In some aspects the reference is a physical map of a nucleic acid from a non-diseased cell. In some aspects the reference is a physical map of a nucleic acid from a diseased cell. In some aspects the reference is a physical map of a nucleic acid from a cell exhibiting a phenotype of interest. In some aspects the reference is derived from a nucleic acid sequence. In some aspects the nucleic acid sequence is a genomic nucleic acid sequence.

In some aspects comparing comprises aligning. In some aspects calculating the spatial extent of a region of interest comprises calculating the smallest rectangle inclusive of two or more landmarks. In some aspects calculating the spatial extent of a region of interest comprises calculating the coordinates of an enclosed area containing the landmark whereby the landmark is not closer than 1 um to any point in the periphery. In some aspects calculating the spatial extent of a region of interest comprises calculating the coordinates of an enclosed area that is a fixed distance upstream or downstream of the landmark. In some aspects calculating the spatial extent of a region of interest comprises calculating the coordinates of an enclosed area based on a landmark and scaled by the observed distances between two or more landmarks. In some aspects calculating the spatial extent of a region of interest comprises calculating the coordinates of an enclosed area to be a fixed distance from a landmark and excluding regions devoid of nucleic acids. In some aspects identifying comprises finding regions of the physical map that differ from the Reference. In some aspects identifying comprises finding regions of the physical map that are similar to a specific portion of the Reference.

Similarly disclosed herein are methods of analyzing a nucleic acid that allow rapid general assessment of the nucleic acid to identify a region of interest, followed by a second, in some cases more specific, analysis of a particular region of interest, such as a region of interest identified with the use of the physical map. Various embodiments comprise one or more of generating a physical map of the nucleic acid in no more than 1 second, comparing the physical map to a reference, and generating a second physical map of a portion of the nucleic acid. In some aspects, the second physical map is of higher resolution than the initial physical map.

Also disclosed herein are systems for analyzing a nucleic acid. Some such systems comprise one or more of the following: an open surface to which the nucleic acid is attached (immobilized), a lens for capturing an optical signal indicative of a physical map of the nucleic acid, and an contact probe for determining a characteristic of a subregion of the nucleic acid. In particular, many such systems allow the physical manipulation of a nucleic acid for which a physical map is determined. Some aspects comprise a stored reference physical map and a processing unit to compare the stored reference physical map to a nucleic acid physical map generated from the fluorescence. In some aspects the processing unit is configured to identify a difference between the stored reference physical map to the nucleic acid physical map generated from the optical signal.

Similarly disclosed herein are methods of analyzing a nucleic acid, comprising one or more of the steps of attaching the nucleic acid to a surface; determining a physical map for at least a portion of the nucleic acid; using the physical map to identify a region of interest in the nucleic acid molecule; and subjecting the region of interest on the nucleic acid molecule to a second physical characterization. In some cases the landmark is a previously identified segment of interest, or is indicative of a distal or overlapping region of interest.

Relatedly, disclosed herein are methods of analyzing a population of nucleic acids. In some cases the population is analyzed through a method comprising generating distinct physical maps of members of the population of nucleic acids, and directing a contact probe to a region within at least one physical map, wherein at least one physical map is generated per molecule within the population per second. In some aspects, the physical maps are generated successively, for example using a common resource on one element at a time. Alternately, some such maps may be generated concurrently. Regions of interest are in some cases identified as regions that are not shared in common among various members od the population, such that non-uniform nucleic acid segments are selectively identified for follow-on analysis.

Also disclosed herein are methods of characterizing a region of interest of a nucleic acid molecule. In various aspects, these methods comprise one or more of the steps of attaching the nucleic acid molecule to a surface of at least one point on the nucleic acid; determining a physical map of at least a portion of the nucleic acid molecule; identifying at least one landmark by comparing the physical map of at least a portion of the nucleic acid molecule to a reference; calculating the spatial extent of a region of interest relative to the landmark; and subjecting the region of interest on the nucleic acid molecule to a second physical characterization. In some aspects these elements are performed in their entirety in order as listed.

It is understood that the various methods and systems as disclosed herein in some cases share common or overlapping aspects. Accordingly, an aspect listed for a method or system herein is equally applied to any of the methods or systems disclosed herein, such that any aspect or element of a method or system herein may be understood in relation to any method or system herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### Definitions

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

The terms used in this specification generally have their ordinary meanings in the art, within the context of the invention, and in the specific context where each term is used. Certain terms are discussed below, or elsewhere in the specification, to provide additional guidance to the practitioner in describing the devices and methods of the invention and how to make and use them. It will be appreciated that way. Consequently, alternative language and synonyms may the same thing can typically be described in more than one be used for any one or more of the terms discussed here.

Synonyms for certain terms are provided. However, a recital of one or more synonyms does not exclude the use of other synonyms, nor is any special significance to be placed upon whether or not a term is elaborated or discussed herein. All publications, patent applications, patents, and other references mentioned herein are incorporated by reference. In the case of conflict, the present specification, including definitions, will control. In addition, the materials, methods, and examples are illustrative only and are not intended to be limiting.

The invention is also described by means of particular examples. However, the use of such examples anywhere in the specification, including examples of any terms discussed herein, is illustrative only and in no way limits the scope and meaning of the invention or of any exemplified term. Likewise, the invention is not limited to any particular embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

As used herein, “about” or “approximately” in the context of a number shall refer to a range spanning +/−10% of the number, or in the context of a range shall refer to an extended range spanning from 10% below the lower limit of the listed range to 10% above the listed upper limit of the range.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application.

The use of the term “combination” is used to mean a selection of items from a collection, such that the order of selection does not matter, and the selection of a null set (none), is also a valid selection when explicitly stated. For example, the unique combinations including the null of the set {A,B} that can be selected are: null, A, B, A and B.

Sample. The term “sample,” as used herein, generally refers to a biological sample of a subject which at least partially contains nucleic acid originating from said subject. The biological sample may comprise any number of macromolecules, for example, cellular long nucleic acid molecules. The sample may be a cell sample. The sample may be a cell line or cell culture sample.

The sample may be a CTC (circulating tumor cells) or CFC (circulating fetal cells) sample. The sample can include one or more cells. The sample may be one or more droplets containing a biological material. The sample can include one or more microbes. The biological sample may be a nucleic acid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

DNA. The terms “nucleic acid”, “nucleic acid molecule”, “oligonucleotide” and “polynucleotide”, “nucleic acid polymer”, “nucleic acid fragment”, “polymer” are used interchangeably and refer to a polymeric form of nucleotides of any length, either deoxyribonucleotides or ribonucleotides, or analogs thereof. The terms encompass, e.g., DNA, RNA and modified forms thereof. Polynucleotides may have any three-dimensional structure, and may perform any function, known or unknown. Non-limiting examples of polynucleotides include a gene, a gene fragment, exons, introns, messenger RNAs (mRNA), transfer RNAs, ribosomal RNAs, lncRNAs (Long noncoding RNAs), lincRNAs (long intergenic noncoding RNAs), ribozymes, cDNA, ecDNAs (extrachromosomal DNAs), artificial minichromosomes, cfDNAs (circulating free DNAs), ctDNAs (circulating tumor DNAs), cffDNAs (cell free fetal DNAs), recombinant polynucleotides, branched polynucleotides, plasmids, vectors, isolated DNA of any sequence, control regions, isolated RNA of any sequence and configuration including circular RNA, nucleic acid probes, and primers.

Unless specifically stated otherwise, the nucleic acid molecule can be single stranded, double stranded, or a mixture there-of. For example, there may be hairpin turns or loops. Unless specifically stated otherwise, the nucleic acid molecule may contain nicks.

Long Nucleic Acid Molecule. Unless specifically stated otherwise, a “long nucleic acid fragment” or “long nucleic acid molecule” is double strand nucleic acid of at least 1 kbp in length, and is thus a kind of macromolecule, and can span to an entire chromosome. It can originate from any source, man-made or natural, including single cell, a population of cells, droplets, an amplification process, etc. It can include nucleic acids that have additional structure such as structural proteins histones, and thus includes chromatin. It can include nucleic acid that has additional bodies bound to it, for example labeling bodies, DNA binding proteins, RNA.

Higher Order Nucleic Acid Structure. A “higher order nucleic acid structure”, or “structure”, or “higher order structure” refers to any 2nd, 3rd, or 4th order DNA structure, including any body bound to said nucleic acid molecule. The nucleic acid molecule may be linear or circular. Nucleic acids can have any of a variety of structural configurations, e.g., be single stranded, double stranded, triplex, replication loop or a combination of both, as well as having higher order intra- or inter-molecular secondary/tertiary/quaternary structures, e.g., chromosomal territories, chromosome boundaries, chromosome regions, compartments, Topologically Associating Domains (TAD), chromatin loop and local direct regulatory factors binding, condensing associated loops, cohesin associated loops, guide nucleic acid, argonaut complexes, CRISPR Cas9 complexes, nucleoprotein complexes, insulator complexes, enhancer-promoter complexes, ribonucleic acid (RNA), small interfering RNA (siRNA), micro RNA (miRNA), guide RNA (gRNA), long non-coding RNA (lncRNA), repeat region binding proteins, telomere modification proteins, nucleic acid repair proteins, regulatory factor binding proteins, nucleic acid binding proteins, proteins, histone deacetylase (HDAC), chromatin remodeling protein, methyl-binding protein, transcription factor transcription complexes, bending with kinks of the genomic DNA polymers such as hairpins, replication loops, triple stranded regions, in cis or trans fashion etc. The nucleotides within the nucleic acid may have any combination of epigenomic state including but not limited to such as methylation or acetylation states. The nucleic acid can originate from any source, man-made or natural, including single cell, a population of cells, droplets, an amplification process, etc. In some embodiments, these structures include compounds and/or interactions of nucleic acids and proteins. In some embodiments, these structures include 2D and 3D configurations of the nucleic acid beyond the linear 1D polymer chain. These 2D and 3D configurations can be formed via interactions with proteins, other nucleic acid molecules, or external boundary conditions. Non limiting examples of boundary conditions include a micro or nanofluidic chamber, a well on or in substrate or defined within a fluidic device, a droplet, a nucleus. The nucleic acid can include nucleic acids that has additional structure such as structural proteins including but not limited to such as any regulatory binding sites complexes, enhancer/transcription factor complex and their interaction with a nucleic acid molecule, Cohesins complex SMC (structural maintenance of chromosomes), ATPase subunits (Smc1 and Smc3), non-SMC regulatory subunits (Rad21/Scc1/Mcd1 and SA1/SA2/Scc3), Sgo1, mitotic kinases (pololike kinase 1 (Plk1) and aurora B), protein phosphatase 2A (PP2A), chromosome passenger complex (CPC), topo II decatenation, condesins, CTCF proteins, PDS5 proteins, WAPL proteins, condensin I, condensin II, CAP-G, histones and their derivative complexes, and thus includes chromatin. In some embodiments, higher order structure can include exogenous nuclei acid genome integration complex, in particular, an exogenous nuclei acid genome integration complex that comprises viral genome integration complexes or recombinant nucleus acid. In some embodiments, higher order structure can include extrachromosomal episomes physical docking complexes, in particular, where such complexes host chromosomes through binding sites. In some embodiments, the higher order nucleic acid structure comprises extrachromosomal nucleic acid deriving from a host chromosome. All of above, not limiting, could be target of labelling, physical or conformational biomarkers indicating the presence of certain state of genome organization or the shift between the states, that could be associated with pathogenomic consequences.

In particular, higher order nucleic acid structure can refer to the various levels of genome organization contained within a cell nucleus [Jerkovic, 2021], [Kempfer, 2020] either individually, collectively, or a sub-set there-of Such genomic organization starts with linear primary DNA winding around histones to form nucleosomes, which are organized into clutches, each containing ˜1-2 kb of DNA. Nucleosome clutches form chromatin nanodomains (CNDs) ˜100 kb in size, where most enhancer-promoter (E-P) contacts take place. At the scale of ˜1 Mb, CNDs and CCCTC-binding factor (CTCF)-cohesin-dependent chromatin loops form topologically associating domains (TADs) and loop domains. On the higher scale up to 100s of megabases, chromatin segregates into gene-active and gene-inactive compartments (A and B, respectively) and into compartment-specific contact hubs, formation of sister chromatid axes. At the highest topological level, the nucleus is organized into chromosome territories.

Hybridization. As used herein, the terms “hybridization”, “hybridizing,” “hybridize,” “annealing,” and “anneal” are used interchangeably in reference to the pairing of complementary or substantially complementary nucleic acids. Hybridization and the strength of hybridization (i.e., the strength of the association between the nucleic acids) is influenced by such factors as the degree of complementary between the nucleic acids, stringency of the conditions involved, the Tm (melting temperature) of the formed hybrid, and environmental conditions, for example: temperature and pH. “Hybridization” methods involve the annealing of one nucleic acid to another, complementary nucleic acid, i.e., a nucleic acid having a complementary nucleotide sequence.

Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

In the context of this document, where hybridization occurs between nucleic acid strand and a double-stranded nucleic acid molecule, it should be understood that such hybridization is being done under conditions of either partial or full denaturation of the double-stranded nucleic acid molecule, unless otherwise specifically stated.

Labelling Body. A “labelling body” used herein is a physical body that can bind to a nucleic acid molecule, or to a body directly or indirectly bound to a nucleic acid molecule, which can be used to generate a signal that can be detected with interrogation, that differs from a detected signal (or lack there-of) that would be generated by said nucleic acid without said body. A labelling body may be a fluorescent intercalating dye that when bound to nucleic acid, can be used in a fluorescent imaging system to identify the presence of said nucleic acid. In another example, a labelling body may by a compound that binds specifically to methylated nucleotides, and gives a current blockade signal when transported through a nanopore, thus reporting a signal as to said molecule's methylation state. In another example, a fluorescent probe specifically hybridized to a sequence of a nucleic acid, thus providing confirmation with a fluorescent imaging system that the sequence is present on said nucleic acid. In another example, a fluorescent probe specifically binds to a specific protein (e.g.: DNA binding protein), with said protein bound to a long nucleic acid molecule. In some cases, the absence of the labelling body, is itself the signal. In some cases, the signal associated with the labelling body is an attenuation, blocking, displacement, quenching, or modification of a signal from another labelling body. Non-limiting examples include: binding of a dark labelling body to the nucleic acid to displace an existing bond fluorescent body; binding of a dark labelling body to the nucleic acid to block a fluorescent labelling body from binding; quenching a near-by fluorescent labelling body bond to a nucleic acid; directly, or indirectly, reacting with a fluorescent labelling body bond to a nucleic acid to reduce its fluorescence. In some cases, the labelling body is not physically attached to the nucleic molecule at the time of interrogating said nucleic molecule and labelling body. For example, a labelling body may be attached to a nucleic acid molecule via a cleavable linker. At the desired time, the linker is cleaved, releasing said labelling molecule which is then detected by interrogation.

Interrogation. “Interrogation” is a process of assessing the state of a nucleic acid, a long nucleic acid molecule, a higher order nucleic acid structure, a nucleic acid-protein complex, or other bio-molecule with an interrogation system. In some embodiments, the state of nucleic acid is assessed by interrogating the state of at least one labelling body on the nucleic acid by measuring a signal generated directly, or indirectly from the labelling body. It may be a binary assessment, such as the labelling body is present, or not. It may be quantitative, such as how many labelling bodies are present on a molecule. It may be a signal density or intensity along a line, an area, or volume. It may be a physical count, or distance between labelling bodies along the length the molecule.

In some embodiments, interrogation is used to generate a digitized representation of a physical map.

In some embodiments, interrogation is used to assess the physical state of a higher order nucleic acid structure. The physical state of the structure being interrogated may comprise the topology of the molecule such as the presence of a loop structure, a set of hierarchical loop structures, the number of supercoils present in a loop or the degree to which one or more loops from the same or separate molecules are intertwined. The physical state of the structure being interrogated may comprise the accessibility of a region of the nucleic acid to a binding partner or a cis or trans acting factor. The physical state of the structure being interrogated may comprise the presence of partially replicated nucleic acid still in close proximity such as Okazaki fragments or a marker of newly synthesized nucleic acid such as results from a pulse of BrdU. The physical state of the structure being interrogated may comprise the level of cohesin left on metaphase chromosomes that has been manipulated experimentally or affected by genetic anomalies (e.g., by depleting either cohesin itself or Wap1), the resulting chromatids display substantially different lengths and shapes, becomes a quantitatively measurable biomarkers indicating of certain pathological states (Losada et al. 2005; Gandhi et al. 2006; Shintomi and Hirano 2009). The physical state of the structure being interrogated may comprise the amount, ratio, and distribution of condensins I and II in these chromatids. The physical state of structure being interrogated may comprise dynamic changes in genome organization, as in Cohesin release and sister chromatid resolution.

In some embodiments, the signal being interrogated may be fluorescent, photoluminescent, electro-magnetic, electrical, magnetic, physical, chemical, exhibit plasmon resonance or enhance raman signals by means of surface enhanced plasmon resonance.

The signal being interrogated may be analog or digital in nature. For example, the signal may be an analog density profile of the labelling body along the length of the nucleic acid in which the signal measured originates from multiple labelling bodies. In some embodiments, the state of the nucleic acid is directly interrogated without a labelling body, for example direct interrogation of long nucleic acid molecules in a cell via phase microscopy, or direct interrogation of nucleic acid via a current blockade nanopore. Non exhaustive examples of different interrogation methods that may be used an interrogation systems either separately, or in combination include fluorescent imaging, bright-field imaging, dark-field imaging, phase contrast imaging, epi-florescent imaging, total internal reflection fluorescence imaging, nearfield/evanescent field imaging, a wave guide, a zero mode waveguide, plasmonic signaling, confocal, scattering, light sheet, structured illumination, stimulated emission depletion, super resolution, stochastic activation super resolution, stochastic binding super resolution, multiphoton, nanopore sensing of a current, voltage, power, capacitive, inductive, or reactive signal (either column blockade through the pore, and tunneling across the pore), chemical sensing (eg: via a reaction), physical sensing (eg: interaction with a sensing probe), SEM, TEM, STM, SPM, AFM. In addition, combinations of different labelling bodies and interrogation methods are also possible. For example: fluorescent imaging of an intercalating dye on a nucleic acid, while translocating said nucleic acid through a nanopore and measuring the pore current.

Interrogation System. Used herein, “Interrogation System” is an automated, or semi-automated system for interrogating the sample. In some embodiments, whereby the sample is interrogated while within or on a fluidic device, the interrogation system interfaces with the fluidic device and controls the operation of the fluid device. In some embodiments, the interrogation system comprises a multitude of separate systems that together can be coordinated by a controller or user. For example, an instrument for loading sample into a fluidic device, an instrument for flowing said sample in said fluidic device, an instrument for imaging said sample in said fluidic device, a controller for operating software for analysis of said imaging data. In some embodiments, the interrogation system comprises an integration of all or a sub-set of systems.

In some embodiments whereby a sample is contained within, or on, a fluidic device, operation of the device by the interrogation system can comprise: manipulating the physical position and conformation of the package or long nucleic acid molecule via the application of external forces on said bodies; exposing the package or long nucleic acid molecule to an environmental condition or reagent for a time period; optically interrogating the static or dynamic configuration of the package or long nucleic acid molecule to facilitate analysis of their composition or as part of a feedback system to control operation of the device; extracting desired packages or long nucleic acid molecules from the device. The fluidic device and interrogation system can interface in a number of ways. A non-exhaustive list includes: fluidic ports (both open and sealed), electrical terminals, optical windows, mechanical pads, heat pipes or sinks, inductance coils, fluid dispensing, surface scanning probes. A non-exhaustive list of potential functions the interrogation system may perform on the device include: temperature monitoring, applying heat, removing heat, applying pressure or vacuum to ports, measuring vacuum, measuring pressure, applying a voltage, measuring a voltage, applying a current, measuring a current, applying electrical power, measuring electrical power, exposing the device to focused and/or unfocused electromagnetic waves, collecting the electromagnetic waves light generated or reflected from the device, in far or near-fiend setting, creating and measuring a temperature, electromagnetic force, surface energy or chemical concentration differential or gradient, dispensing liquid into a device well or port, or on the device surface, contacting the device surface or entity on the device surface with a contact probe (for example: an AFM tip).

In some embodiments, confirmation of the presence of a long nucleic acid molecule in a certain region of a fluid device and control over its physical position within said device is controlled by the interrogation system using a feedback controller system. Detection of the long nucleic acid molecule is via detection of at least one interrogated signal. In the preferred embodiment, the signal is an electromagnetic signal originating from a labelling body bound to said long nucleic acid molecule. In one embodiment, the control instrument feedback control system at least in part utilizes as input information the identification of a physical map profile within the long nucleic acid molecule, or absence of a physical map profile within the molecule.

In some embodiments, the interrogation system comprises localized computational processing modules within the system, adjacent computational processing modules via a direct communication connection, external computational processing modules via a network connection, or combination there-of Various examples of computational processing modules include: a PC, a micro-controller, an application specific integrated micro-chip (ASIC), a field-programmable gate array (FPGA), a CPU, a GPU, System on Chip, a network server, cloud computing service, or combinations there-of.

The interrogation system may include at least one fluidic dispensing tip that is capable of dispensing fluid drops at the desired x,y,z coordinates on the surface of the device, and in some embodiments, extracting fluid drops at the desired x,y,z coordinates on the surface of the fluidic device. Fluid dispensing and extracting may be in volumes of microliters, nanoliters, picoliters, femtoliters, or attoliters.

The interrogation system may be able to illuminate multiple light sources simultaneously, or in series, and be able to image multiple colors simultaneously, or in series. If imaging multiple colors simultaneously, this may be done on different cameras, on a single camera but different regions of the sensor array, or on the same sensor of the same camera. In some embodiments, the wavelength of light illuminated by the control instrument is chosen so as to interact with the sample, the sample labelling body, or a functionalized surface in some way. Non limiting examples include: photo-cleaving of the nucleic acid, photo-cleaving photo-cleavable linkers, manipulating optical tweezers, activating photo-activated reactions, de-protecting photolabile protecting groups, IR thermal heating.

Unless specifically stated otherwise, in this document any interrogation of a long nucleic acid molecule by an interrogation system comprises the embodiment where-by at least a portion of the long nucleic molecule is bound with at least one labelling body that comprises an intercalating fluorescent dye, and the interrogation system comprises an optical fluorescent imaging system.

Sequence. The term “sequence” or “nucleic acid sequence” or “oligonucleotide sequence” refers to a contiguous string of nucleotide bases and in particular contexts also refers to the particular placement of nucleotide bases in relation to each other as they appear in an oligonucleotide.

Sequencing can be performed by various systems currently available, such as, with limitation, a sequencing system by Illumina, Pacific Biosciences, Oxford Nanopore, Life Technologies (Ion Torrent), BGI.

Structural variation. As used herein, “structural variation”, “structural variant”, or “SV” is the variation in structure of an organism's chromosome with respect to a genomic reference. These variations include a wide variety of different variant events, including insertions, deletions, duplications, retrotransposition, translocations, inversions short and long tandem repeats, rearrangements, and the like. These structural variations are of significant scientific interest, as they are believed to be associated with a range of diverse genetic diseases. In general, the operational range of structural variants includes events >50 bp, while the “large structural variations” typically denotes events >1,000 bp or more. The definition of structural variation does not imply anything about frequency or phenotypical effects.

Reference. A “genomic reference” or “reference” is any genomic data set that can be compared to or aligned to another genomic data set. Any data formats may be employed, including but not limited to sequence data, karyotyping data, methylation data, genomic functional element data such as cis-regulatory element (CRE) map, primary level structural variant map data, higher order nucleic acid structure data, physical mapping data, genetic mapping data, optical mapping data, raw data, processed data, simulated data, signal profiles including those generated electronically or fluorescently. A genomic reference may include multiple data formats. A genomic reference may represent a consensus from multiple data sets, which may or may not originate from different data formats. The genomic reference may comprise a totality of genomic information of an organism or model, or a subset, or a representation. The reference may be a representation of a portion of a genome. The reference may be a representation of a portion of chromosome. The reference may be a representation of a gene or portion thereof. The reference may be a representation of a regulatory region or portion thereof. The reference may be a representation of a TAD, domain, region or portion thereof. The genomic reference may be an incomplete representation of the genomic information it is representing.

The genomic reference may be derived from a genome that is indicative of an absence of a disease or disorder state or that is indicative of a disease or disorder state. Moreover, the genomic reference (e.g., having lengths of longer than 100 bp, longer than 1 kb, longer than 100 kb, longer than 10 Mb, longer than 1000 Mb) may be characterized in one or more respects, with non-limiting examples that include determining the presence (or absence) of a particular feature, a particular haplotype, a particular genetic variations, a particular structural variation, a particular single nucleotide polymorphism (SNP), and combinations thereof, referring not only to being present or absent from the genomic reference in its entirety, but also from a particular region of genomic reference, as defined by the neighboring genomic content. Moreover, any suitable type and number of characteristics of the genomic reference can be used to characterize the sample nucleic acid, as derived (or not derived) from a nucleic acid indicative of the disorder or disease based upon whether or not it displays a similar character to the reference.

In some cases, the genomic reference is a physical map. This can be generated in any number of ways, including but not limited to: raw single molecule data, processed single molecule data, a digitized representation of a physical map generated from a sequence or simulation, a digitized representation of a physical map generated by assembling and/or averaging multiple single molecule physical maps, or combination there-of. For example, based on a known, or partially known sequence, a simulated digitized physical map can be generated based on the method of generating a physical map used. In an embodiment where-by the physical map comprises labelling bodies at known sequences, a discrete ordered set of segment lengths in base-pairs can be generated. In an embodiment where-by the physical map comprises a continuous analog signal of labeling signal density along the sequence length, in base-pairs based on simulated local hydrogen bonds dissociation kinetics between the double helices, in chemical moiety modification, regulatory factor association or structural folding patterns based on nucleotide sequence and predicted functional element database maps.

In some cases, the genomic reference is data obtained from microarrays (for example: DNA microarrays, MMChips, Protein microarrays, Peptide microarrays, Tissue microarrays, etc), or karyotypes, or FISH analysis. In some cases, the genomic reference is data obtained from proximity 3D Mapping technologies or 3D physical mapping technologies.

In some cases, characterizations of the comparison or alignment with the genomic reference may be completed with the aid of a programmed computer processor. In some cases, such a programmed computer processor can be included in a computer control system.

Alignment. An “alignment” is any process where-by genomic information that can be represented as a collection of information along at least one axis is statistically compared to at least one other genomic information that can be represented as a collection of information along at least one axis. In the preferred embodiment, the statistical comparison results in the orientation and overlap of the two genomic information that provides the best global similarity within their respective axis(axes). In the preferred embodiment, the statistical comparison output provides a similarity score or confidence score associated with the best global similarity, along with coordinates within their respective axis(axes) of the best global similarity. The genomic information can be raw, processed, digitized, in-silico, or simulated. Examples of different axis can include base-pairs, k-mers, domains, molecule length, molecule depth, molecule width, physical dimensions (for example: nm).

For embodiments where-by the genomic information being aligned is a sequence, the similarity score can be determined in a number of different manners including BLAST, available over the world wide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments, with a restricted affine gap penalty model. See Meth. Mol. Biol. 70: 173-187 (1997). Also, the GAP program using the Needleman and Wunsch alignment method can be utilized to align sequences using a general class of gap models. See J. Mol. Biol. 48: 443-453 (1970).

In some embodiments, a subject computer program will analyze genomic information by comparing two or more physical maps with each other to generate a similarity score. In some embodiments, at least one physical map is a digitized representation of an interrogated physical map of a long nucleic acid molecule. In some embodiments, at least one physical map is a digitized representation of a reference. In some embodiments, at least one physical map is in a digitized representation of a simulated long nucleic acid molecule. For example, in some embodiments, a subject computer program will compare a first physical map with at least a second physical map; and will compute their alignment. The similarity between a first physical map and at least a second physical maps will in some embodiments be computed by aligning the first physical map and the at least second physical map with one another; and recording a similarity score value of the best alignment. The score function will in some embodiments be the likelihood that the first physical map and the at least second physical map(s) are derived from the same molecule, or the same genome. The likelihood may be derived from a Bayesian prior modeling various noise processes, where noise processes include, e.g., sizing error, false negative, false positive, etc. The alignment may be optimized using a dynamic programming algorithm. In some embodiments, the similarity between a first physical map and at least a second physical map will in other embodiments be computed by comparing the output of a heuristic function applied to the physical map.

Physical Mapping. “Physical mapping” or “mapping” of nucleic acid comprises a variety of methods of extracting genomic, epigenomic, functional, or structural information from a physical fragment of long nucleic acid molecule, in which the information extracted can be associated with a physical coordinate on the molecule. As a general rule, the information obtained is of a lower resolution than the actual underlying sequence information, but the two types of information are correlated (or anti-correlated) spatially within the molecule, and as such, the former often provides a ‘map’ for sequence content with respect to physical location along the nucleic acid. In some embodiments, the relationship between the map and the underlying sequence is direct, for example the map represents a density of AG content along the length of the molecule, or a frequency of a specific recognition sequence. In some embodiments, the relationship between the map the underlying sequence is indirect, for example the map represents the density of nucleic acid packed into structures with proteins, which in turn is at least partially a function of the underlying sequence. In some embodiments, the physical map is a linear physical map, in which the information extracted can be assigned along the length of an axis, for example, the AT/CG ratio along the major axis of long nucleic acid molecule. In some embodiments, the “linear physical map” or “1D physical map” is generated by interrogating labelling bodies that are bound along an elongated portion of a long nucleic acid molecule's major axis. For clarity, a string occupying 3D space in a coiled state can be represented as straight line, and thus extracted values along the 3D coil, can be represented as binned values along a 1D representation of the string, and thus constitute a linear physical map. In some embodiments, the physical map is a “2D physical map”, in which the information extracted can be assigned within a plane that comprises the molecule, for example: karyotyping. In some embodiments, the physical map is a “3D physical map”, in which the information extracted can be assigned in 3D volume in which the molecule occupies. For example, tagging with super-resolution techniques to identify in (x,y,z) space the location of the tag within the chromosome as demonstrated with OligoFISSEQ [Nguyen, 2020], or in-situ genome sequencing [Payne, 2020].

In some embodiments, the physical map comprises the physical pattern of higher order nucleic structures within the long nucleic molecule. In some embodiments, the physical map comprises the locations of TADs within the molecule. In some embodiments, the physical map comprises the locations of histones within the molecule. In some embodiments, the physical map comprises the locations of loops within the molecule. In some embodiments, the physical map comprises the locations of knots within the molecule. In some embodiments, the physical map comprises the locations of binding factors within the molecule.

In some embodiments, the physical map of a long nucleic acid molecule comprises multiple physical map types that are merged into a single physical map. For example, a long nucleic acid molecule with a fluorescent physical map that correlates with the localized AT density along the length of the molecule merged with a second physical map that indicates the locations of loops along the length of the molecule.

The first and most widely used form of physical mapping is karyotyping, where-by metaphase chromosomes are treated with a stain process that preferentially binds to AT or CG regions, thus producing ‘bands’ that correlate with the underlying sequence as well as the structural and epigenomic patterns of the nucleic acid [Moore, 2001]. However, the resolution of such a process with respect to nucleotide sequence is quite poor, about 5-10 Mbp, due to the condensed nature of nucleic acid being imaged. More recent methods of using linear mapping of elongated interphase genomic DNA have been generated by imaging nucleic acid digested at known restriction sites [Schwartz, 1988, U.S. Pat. No. 6,147,198] (eg: see FIG. 1(A)), imaging attached fluorescent probes at nicking sites [Xiao, 2007] (eg: see FIG. 1(B)), imaging the fluorescent signature of a nucleic acid molecule's methylation pattern [Sharim, 2019], imaging the fluorescent signature of a chromatin's histone [Riehn, 2011], electrical detection of bound probes to a nucleic acid through a sensor [Rose, 2013, 2014/0272954], and electrical detection of the methylation signature on a nucleic acid using a nanopore sensor [Rand, 2017].

Another method of linear physical mapping is to measure the AT/CG relative density or local melting temperature along the length of an elongated nucleic molecule (eg: see FIG. 1(C)). Such a signal can either be used to compare or align against other similar maps, or against a map generated in-silico from sequence data. There are many ways of generating such a signal. For example, the signal can be fluorescent or electrical in nature. Nucleic acid can be uniformly stained with an intercalating dye, and then partially melted resulting in the relative loss of dye in regions of rich AT content [Tegenfeldt, 2009, U.S. Pat. No. 10,434,512]. Another method is to expose double stranded nucleic acid to two different species that compete to bind to the nucleic acid. One species is non-fluorescent and preferentially binds to AT rich regions, while the other species is fluorescent and has no such bias [Nilsson, 2014]. Yet another method is to use two different color dyes that differentially label the AT and CG regions.

Mapping using such non-condensed interphase nucleic acid polymer strands has improved upon the resolution of the primary sequence information, however the maps were stripped of any native structural folding or bound supporting proteins information and are often extracted from bulk solution of pooled samples with many potentially heterogeneous cells. Recently, 3D physical maps have been demonstrated where-by tags attached to chromosomes as specific locations are interrogated directly or indirectly to determine their relative position within the chromosome in 3D space (see [Jerkovic, 2021] for a review of the various methods). These methods can include super resolution microscopy methods such as SIM, SMLM, and STED, Oligopaint FISH methods, multiplexed oligopaint FISH methods, and OligoFISSEQ methods. In addition, also included are in-situ sequencing methods such as OligoFISSEQ [Nguyen, 2020]. Note, in this document, “3D physical Map(ping)” is different from “Proximity 3D Map(ping)”, which is defined elsewhere in this document.

FIG. 1 demonstrates a variety of different embodiments for generating and interrogating a long nucleic acid molecule linear physical map. In FIG. 1(A), a physical map of a long nucleic acid molecule 104 is generated by cleaving the molecule at particular sequence sites (eg: recognition sites for restriction enzymes) thus resulting in gaps 105 where the cleaving event took place. Along the length of a molecule, a dye is attached non-specifically (eg: using an intercalating dye) such that child molecules from the originating the parent molecule can be interrogated to generate a signal 101 that follows the physical length (0106) of the parent molecule. The signal can then be used determined the lengths and order of the individual child molecules {103-x}, and thus generating the parent molecule's physical map. In most embodiments of this method, the parent molecule is combed onto a surface and then cleaved, so as to maintain physical proximity and relative order of the child molecules. However, such an embodiment could also be implemented in at least a partially elongated state within an elongating channel of a confined fluidic device such that the order of the child molecules can be interrogated [Ramsey, 2015, U.S. Pat. No. 10,106,848]. In some embodiments, a mixture of different cleaving sites may be used simultaneously.

In FIG. 1(B), a physical map of a long nucleic acid molecule 114 is generated by sparsely binding label bodies 115 along the length of the molecule, with the binding sites correlated (or anti-correlated) with a set of specific target(s). In some methods, the labelling body is bound directly to a sequence motif target. In some methods, the labelling body generating a signal is bound indirectly via a process, for example: a sequence specific nick is generated, followed by incorporation of nucleotides starting at the nick site, some of which may be capable of generating a signal. The long nucleic acid molecule with labelling bodies is interrogated, generating signals 111 from the label bodies 115 along the physical length of the molecule 116. The distance between the signals, a collection of lengths and orders {113-x} then represents the molecule's physical map. In some embodiments, further information can be generated by also interpreting the relative magnitudes of the signals 112 from the various labelling sites. When fluorescent interrogation is used, different color labelling bodies can be used to represent different specific sites.

In FIG. 1(C), a physical map of a long nucleic acid molecule 124 is generated by densely binding labelling bodies 125 along the length of the molecule, such that the binding pattern correlates (or anti-correlates) with the underlying physical sequence content of the molecule. For example, the relative AT/CG content, or the relative melting temperature, or the relative density of methylated CGs. Due to the dense nature of the labelling bodies in this method, the physical map is not a collection of lengths and orders, but rather an analog signal 121 that varies in intensity along the physical length of the molecule 126.

The method of interrogation to generate a physical map is typically fluorescent imaging, however different embodiments are also possible, including a scanning probe along the length of a combed molecule on a surface, or a constriction device that measures the coulomb blockade current through or tunneling current across the constriction as the molecule translocate through.

Unless specifically stated otherwise, a physical map refers to any of the previously mentioned methods, including combinations there-of. For example, a long nucleic acid molecule may have a physical map generated from the AT/TCG density with a fluorescent labelling body along the length of the molecule, and then also have a physical map generated from the methylation profile along the length of the molecule by constriction device as the molecule is transported through said constriction device.

Elongated Nucleic Acid. The majority of linear physical mapping methods that use fluorescent imaging or electronic signals to extract a signal related to the underlying genomic, structural, or epigenomic content employ some form of method to at least locally ‘elongate’ the long nucleic acid molecule such that the resolution of the physical mapping in the region of elongation can be improved, and disambiguates reduced. A long nucleic acid molecule in its natural state in a solution will form a random coil. Thus, a variety of methods have been developed to ‘uncoil’ and elongate the molecule.

By binding a portion of long nucleic acid molecules on a functionalized solid surface, the molecule is elongated by flowing a solution and ultimately pulled taut, coming into full contact with the substrate surface [Bensimon, 1997, U.S. Pat. No. 7,368,234], a technique typically called ‘combing’ DNA. Alternatively, there are other long polymer elongation methods such as fluid flow induced elongation with ends anchoring on surface [Gibb, 2012], aqueous solution hydrodynamic focusing by laminar flows [Chan, 1999, U.S. Pat. No. 6,696,022], linearization by confining nanochannels [Tegenfeldt, 2005], long nucleic acid molecules in microfluidic device pulled by two angled opposing externally applied forces in a presence of physical obstacle features[Volkmuth, 1992], molecules hydrodynamically trapped in a fluidic device by simultaneously exposed to two opposing externally applied forces [Tanyeri, 2011].

Most of the time, the elongation state of at least a portion of the long nucleic acid molecule has to be sustained by an external force before otherwise returning to its natural random coiled state, unless at least a portion of the nucleic acid is retained in the elongated state by physical confinement without a sustaining external force [Dai, 2016].

Unless specifically stated otherwise, an ‘elongated’ or ‘partially elongated’ nucleic acid is a long nucleic acid fragment for which at least one segment of the major axis of the molecule comprising at least 1 kb can be projected against a 2D plane, and does not overlap with itself. For clarity, for embodiments where-by long nucleic acid includes additional structure, for example as when the nucleic acid is contained in chromatin, compacted with histones, the major axis refers to the larger chromatin molecule, not the nucleic acid strand itself. Therefore statements in this disclosure such as “along the length of the molecule” when referring to long nucleic acid molecules, refers to along the length of the major axis.

Proximity 3D mapping. In this document, “proximity 3D mapping” refers to protocols that involve capturing the proximity relationship of at least two strands of nucleic acid, either of the same chromosome or not, by crosslinking them together directly or indirectly. For reference [Kempfer, 2020], and [Szabo, 2019] reviews these various techniques, of which a non-exhaustive list includes the following: 3C, 4C, 5C, Hi-C, TCC, PLAC-seq, ChIA-PET, Capture-C, C-HiC, Single-Cell HiC, GAM, SPRITE, ChIA-Drop.

Barcode. As used herein a “barcode” is a short nucleotide sequence (e.g., at least about 4, 6, 8, 10, 12, 14, 16, 18, 20, 25, 30, 35 nucleotides long) that encodes information. The barcodes can be one contiguous sequence or two or more noncontiguous sub-sequences. Barcodes can be used, e.g., to identify molecules in a partition or a bead, or a body to which an oligonucleotide is attached. In some embodiments, a bead-specific barcode is unique for that bead as compared to barcodes in oligonucleotides linked to other beads. In another example, a nucleic acid from each cell can be distinguished from nucleic acid of other cells due to the unique “cellular barcode.” Such partition-specific, cellular, or bead barcodes can be generated using a variety of methods. In some cases, the partition-specific, cellular, or particle barcode is generated using a split and mix (also referred to as split and pool) synthetic scheme, for example as described in [Agresti, 2014, 2016/0060621]. More than one type of barcodes can in some embodiments be in the oligonucleotides described herein.

In some embodiments, the information associated with the barcode may be an identification of a single, a particular, a type, a sub-set, a specific selection, a random selection, a group of body, where the body may be a molecule, a higher-order nucleic acid structure, an organelle, a sample, a subject. In some embodiments, the information associated with the barcode may be a process, a time-stamp, a location, a relationship with another body and/or barcode, an experiment id, a sample id, or an environmental condition. In some embodiments multiple information content may be stored in the barcode, using any encoding technique.

In some embodiments the barcode is single strand. In some embodiments the barcode is double-stranded. In some embodiments, the barcode has both single and double strand components. In some embodiments the barcode is at least partially comprised of 2D and/or 3D structures, for example hairpins or a DNA origami structure.

In some embodiments, the information encoded in the barcode is done using error checking and/or error-correcting techniques to ensure the validity of the information stored within. For example, the use of hamming codes. In some cases where multiple information content is stored in the barcode, the separate pieces of information are encoded separately with their respective nucleotides within the barcodes. In other cases, the nucleotides can be shared using an encoding scheme. In some cases, compression techniques can be used to reduce the number of nucleotides needed.

In some embodiments, the information encoded in the barcode includes uniquely identifying the molecule to which it is conjugated. These types of barcodes are sometimes referred to as “unique molecular identifiers” or “UMIs”. In still other examples, primers can be utilized that contain “partition-specific barcodes” unique to each partition, and “molecular barcodes” unique to each molecule. After barcoding, partitions can then be combined, and optionally amplified, while maintaining “virtual” partitioning based on the particular barcode. Thus, e.g., the presence or absence of a target nucleic acid comprising each barcode can be counted or tracked (e.g. by sequencing) without the necessity of maintaining physical partitions.

The length of the barcode sequence determines how many unique barcodes can be differentiated. For example, a 1 nucleotide barcode can differentiate 4, or fewer, different samples or molecules; a 4 nucleotide barcode can differentiate 256 samples or less; a 6 nucleotide barcode can differentiate 4096 different samples or less; and an 8 nucleotide barcode can index 65,536 different samples or less.

In some embodiments, the barcode sequences are designed or randomly generated using a selection software for choosing barcodes that are: without hairpin, or containing even base composition (15%-30% A,T,G and C), or without homopolymers (default allows 3 bases of same nucleotides), or without simple repeats, or without low complexity sequences, or not identical to common vector or adaptor sequences. Furthermore, barcodes can be designed to be unique even if there are 3 mismatch sequencing errors.

Barcodes are typically synthesized and/or polymerized (e.g., amplified) using processes that are inherently inexact. Thus, barcodes that are meant to be uniform (e.g., a cellular, particle, or partition-specific barcode shared amongst all barcoded nucleic acid of a single partition, cell, or bead) can contain various N−1 deletions or other mutations from the canonical barcode sequence. Thus, barcodes that are referred to as “identical” or “substantially identical” copies can in some embodiments include barcodes that differ due to one or more errors in, e.g., synthesis, polymerization, or purification errors, and thus can contain various N−1 deletions or other mutations from the canonical barcode sequence. However, such minor variations from theoretically ideal barcodes do not interfere with the methods, compositions, and kits described herein. Therefore, as used herein, the term “unique” in the context of a particle, cellular, partition-specific, or molecular barcode encompasses various inadvertent N−1 deletions and mutations from the ideal barcode sequence. In some cases, issues due to the inexact nature of barcode synthesis, polymerization, and/or amplification, are overcome by oversampling of possible barcode sequences as compared to the number of barcode sequences to be distinguished (e.g., at least about 2-, 5-, 10-fold or more possible barcode sequences), or by using error correction encoding techniques. The use of barcode technology is well known in the art, see for example [Shiroguchi, 2012] and [Smith, 2010]. Further methods and compositions for using barcode technology include those described in [Agresti, 2014, 2016/0060621].

In some embodiments, at least a portion of the barcode can also be used as a primer binding site. In some embodiments, the primer binding site is for a PCR primer. In some embodiments, all barcodes that form a set of unique barcodes contain within said barcodes a globally identical primer binding site, such that a single primer sequence can be used to bind to all barcodes. In some embodiments, the primer will be the complement sequence of the primer binding site. In other embodiments, the primer will be the same sequence as the primer binding site, as the primer will bind to a previously amplified product of the original primer binding site. In some embodiments, there may be a combination.

In addition, in some embodiments, at least a portion of the barcode can also be used a primer.

Binding. “Binding”, “bound”, “bind” as used herein generally refers to a covalent or non-covalent interaction between two entities (referred to herein as “binding partners”, e.g., a substrate and an enzyme or an antibody and an epitope). Any chemical binding between two or more bodies is a bond, including but not limited to: covalent bonding, sigma bonding, pi ponding, ionic bonding, dipolar bonding, metallic bonding, intermolecular bonding, hydrogen bonding, Van der Waals bonding. As “binding” is a general term, the following are all examples of types of binding: “hybridization”, hydrogen-binding, minor-groove-binding, major-groove-binding, click-binding, affinity-binding, specific and non-specific binding. Other examples include: Transcription-factor binding to nucleic acid, protein binding to nucleic acid.

Specifically Bind. As used herein, the terms “specifically binds” and “non-specifically binds” must be interpreted in the context for which these terms are used in the text. For example, a body may “specifically bind” to a nucleic acid molecule but have no significant preference or bias with respect the underlying sequence of said nucleic acid molecule over some genomic length scale and/or within some genomic region. As such, in the context of molecule's sequence, the body “non-specifically binds” to said nucleic acid molecule.

When in the context of binding between physically distinct molecules, “Specific binding” typically refers to interaction between two binding partners such that the binding partners bind to one another, but do not bind other molecules that may be present in the environment (e.g., in a biological sample, in tissue) at a significant or substantial level under a given set of conditions (e.g., physiological conditions).

Preferentially Bind. The term “preferentially binds” means that in comparison between at least two different binding sites (the sites can be on the same entity, or can be physically different entities), there is a non-zero probability of binding between a certain body and both sites, however conditions can exist in which the probability of binding of the certain body is preferable at one site over another.

Genomic Information. The term “genomic information” or “genomic data” here includes any information content obtained directly or indirectly from the interrogation of a nucleic acid molecule that relates directly or indirectly to the underlying conventional genomic and epigenomic content of said molecule. Such information may include at least a portion of sequence information, the orientation (5-prime, 3-prime) of the molecule with respect to said molecule's physical environment or the molecule itself, where the positional reference within the molecule may be physical length, sequence, a physical map along the molecule, or at least one labeling body bound to said molecule. The physical position of a base, or sequence with respect to said molecule's physical environment, or the molecule itself, where the positional reference within the molecule may be physical length, sequence, a physical map along the molecule, or at least one labeling body bound to said molecule. The physical position of a structural variant with respect to said molecule's physical environment, or the molecule itself, where the positional reference within the molecule may be physical length, sequence, a physical map along the molecule, or at least one labeling body bound to said molecule. The physical position of a higher order nucleic acid structure with respect to said molecule's physical environment, or the molecule itself, where the positional reference within the molecule may be physical length, sequence, a physical map along the molecule, or at least one labeling body bound to said molecule. The physical position of epigenetic data with respect to said molecule's physical environment, or the molecule itself, where the positional reference within the molecule may be physical length, sequence, a physical map along the molecule, or at least one labeling body bound to said molecule. The physical position of epigenetic data with respect to said molecule's physical environment, or the molecule itself, where the positional reference within the molecule may be physical length, sequence, a physical map along the molecule, or at least one labeling body bound to said molecule. The physical position of a labelling body bound to said molecule with respect to said molecule's physical environment, or the molecule itself, where the positional reference within the molecule may be physical length, sequence, a physical map along the molecule, or at least one additional labeling body bound to said molecule. The physical position of a body bound to said molecule with respect to said molecule's physical environment, or the molecule itself, where the positional reference within the molecule may be physical length, sequence, a physical map along the molecule, or at least one labeling body bound to said molecule.

Examples can include the relative position of a gene within a molecule as identified by an analysis of a physical map alignment to a reference along the length of said molecule measured in base-pairs, or the relative position of a cohesin loop along the length of said molecule measured in physical length distance, or the relative position of a methylation pattern with respect to the underlying sequence of the molecule.

In some embodiments, the genomic information may be the relative position of at least two independent portions of genomic information with respect to each other within the molecule, or some other physical reference location or fiducial. For example, the relative position of a TAD with respect to a labelling body within the molecule, or some other physical reference location or fiducial.

Substrate. As used herein, the term “substrate” is intended to mean a solid or semi-solid support that can serve as the foundation for the definition of features. Non limiting examples of features include wells, immobilized molecules, pillars, channels, pits. The features can randomly positioned on the substrate, or patterned. A substrate as provided herein can be modified to accommodate attachment of biopolymers by a variety of methods well known to those skilled in the art. Exemplary types of substrate materials include glass, modified glass, functionalized glass, inorganic glasses, silicon, silicon di-oxide, silicon nitride, quartz, metals, mica, fused silica, microspheres, including inert and/or magnetic particles, polysaccharides, nitrocellulose, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene polycarbonate, or combinations thereof.

Those skilled in the art will know or understand that the composition and geometry of a substrate as provided herein can vary depending on the intended use and preferences of the user. Therefore, although planar substrates such as slides, chips or wafers are often exemplified herein, given the teachings and guidance provided herein, those skilled in the art will understand that a wide variety of other substrates exemplified herein or well known in the art also can be used in the methods and/or compositions herein.

The substrate may comprise of multiple substrates that are physically connected, for example using any combination of bonding mechanism, an adhesive, a film, a vacuum.

The substrate may include various combinations of coatings.

The substrate may have a patterned surface. The patterning may be additive or subtractive in nature, or combination of both.

The substrate may comprise a component of a microfluidic device or a flow-cell.

A substrate may be a film, which itself, may be in contact with another substrate.

A substrate can be of any desired shape. For example, a substrate can be typically a thin, flat shape (e.g., a square or a rectangle or oval). In some embodiments, a substrate structure has rounded corners (e.g., for increased safety or robustness). In some embodiments, a substrate structure has one or more cut-off corners (e.g., for use with a slide clamp or cross-table). In some embodiments, where a substrate structure is flat, the substrate structure can be any appropriate type of support having a flat surface (e.g., a chip or a slide such as a microscope slide).

In some embodiments where the substrate is modified to contain one or more features, including but not limited to, wells, projections, ridges, features, or markings, the features can include physically altered sites. For example, a substrate modified with various features can include physical properties, including, but not limited to, physical configurations, magnetic or compressive forces, chemically functionalized sites, chemically altered sites, surface energy altered sites, hydrophobic/hydrophilic altered sites, and/or electrostatically altered sites.

In some embodiments, a substrate includes one or more markings on a surface of a substrate, e.g., to provide guidance for correlating spatial information with the characterization of interest. For example, a substrate can be marked with a grid of lines (e.g., to allow the size of objects seen under magnification to be easily estimated and/or to provide reference areas for counting objects). In some embodiments, fiducial markers can be included on a substrate. Such markings can be made using techniques including, but not limited to, printing, etching, sand-blasting, and depositing on the surface.

In some embodiments, a fiducial marker can be present on a substrate to provide orientation of the sample with features on the substrate, or the substrate itself.

Functionalized. A “functionalized” surface is a surface of a substrate that has been modified or engineered such as by certain chemicals, or macromolecules to elicit certain desired properties. For example: to bind specifically or non-specifically to a macromolecule, or to provide a reagent.

Immobilized. As used herein, the term “immobilized” when used in reference to molecules in direct or indirect attachment to a substrate via covalent or non-covalent bond(s) or stationery state by physical confinement or held stationery by an external force. Indirectly attached to the substrate may be via at least one additional intermediary molecule or body. In certain embodiments, covalent attachment can be used, but all that is required is that the molecules remain co-localized to the substrate under conditions in which it is intended to use. Non limiting examples include the entire molecule may be held stationary with respect to the substrate, or a portion of the molecule held stationary with respect to the substrate, while the remainder of the molecule has limited freedom of movement, or the molecule is indirectly attached to the substrate via an intermediary, and the entire molecule has some limited freedom of movement. For example, immobilization of an oligonucleotide to a substrate can occur via hybridization of said oligonucleotide to a secondary oligonucleotide, said secondary oligonucleotide at least partially containing a complementary sequence to the first, and itself immobilized to the substrate.

In certain embodiments, a molecule may be immobilized on a surface via physisorption.

In certain embodiments, molecules can include biomolecules, nucleic acid molecules, proteins, peptides, nucleotides, or any combination thereof.

Certain embodiments may make use of a substrate which has been functionalized, for example by application of a layer or coating of an intermediate material comprising reactive groups which permit covalent attachment to biomolecules, such as polynucleotides.

Exemplary bonding examples include click chemistry techniques, non-specific interactions (e.g. hydrogen bonding, ionic bonding, van der Waals interactions etc.) or specific interactions (e.g. affinity interactions, receptor-ligand interactions, antibody-epitope interactions, avidin-biotin interactions, streptavidin-biotin interactions, lectin-carbohydrate interactions, etc.). Exemplary bonding mechanism are set forth in U.S. Pat. Nos. [Pieken, 1998, U.S. Pat. No. 6,737,236]; [Kozlov, 2003, U.S. Pat. No. 7,259,258]; [Sharpless, 2002, U.S. Pat. No. 7,375,234] and [Pieken, 1998, U.S. Pat. No. 7,427,678]; and US Pat. Pub. No. [Smith, 2004, 2011/0059865], each of which is incorporated herein by reference.

Surface Energy. Surface tension of a fluid is the energy parallel to the surface that opposes extending the surface. Surface tension and surface energy are often used interchangeably. Surface energy is defined here as the energy required to wet a surface. To achieve optimum wicking, wetting and spreading, the surface tension of a fluid is decreased and is less than the surface energy, of the surface to be wetted. The wicking movement of a fluid through the channels of a fluid device occurs via capillary flow. Capillary flow depends on cohesion forces between liquid molecules and forces of adhesion between liquid and walls of channel. The Young/Laplace Equation states that fluids will rise in a channel or column until the pressure differential between the weight of the fluid and the forces pushing it through channel are equal. [Moore, 1962] Walter J. Moore, Physical Chemistry 3rd edition, Prentice-Hall, 1962, p. 730:

$$Δ p = ( 2 γcosθ ) / r$$

where Δp is the pressure differential across the surface, γ is the surface tension of the liquid, θ is the contact angle between the liquid and the walls of the channel and r is the radius of the cylinder. If the capillary rise is h and ρ is the density of the liquid then the weight of the liquid in the column is πr2ghp or the force per unit area balancing the pressure difference is ghp, therefore:

$$( 2 γcos θ ) / r = gh ρ$$

For maximum flow through capillary channels, the radius of the channel should be small, the contact angle θ should be small and γ the surface tension of the fluid should be large. The theoretical explanation of this phenomenon can be described by the classical model know as Young's Equation:

$$γ SV = γ SL + γ LV cos θ$$

which describes the relationship between the contact angle θ and surface tension of liquid-vapor interface γLV, the surface tension of the solid-vapor interface γSV, and surface tension of the liquid-vapor interface γSL. When the contact angle θ between liquid and solid is zero or so close to 0, the liquid will spread over the solid. A contact angle measurement test is used as an objective and simple method to measure the comparative surface tensions of solids. In general, a material is considered to be hydrophilic when the contact angle in this test is below 90°. If the contact angle is above 90°, the material is considered to be hydrophobic.

Combing. Defined herein, “molecular combining” or “combing” refers to the process of immobilizing at least a portion of a macromolecule, in particular nucleic acid molecules, to a substrate surface, or within a porous film on a substrate surface, such that at least a portion of the macromolecule is elongated in a plane that is substantially parallel to the surface of said substrate. The elongated portion can be fully immobilized to the substrate, or at least of portion of said portion have some degree of freedom. In some embodiments at least a portion of the molecule is elongated within a porous material film parallel to the surface of said substrate, or at least a portion of the molecule is elongated on top of a porous material film parallel to the surface of said substrate, or at least a portion of the molecule is elongated and suspended between two points. In some embodiments, the substrate surface is at least part of a fluidic device.

In one embodiment, a single nucleic acid molecule binds by one or both extremities (or regions proximal to one or both extremity) to a modified surface (e.g., silanised glass) and are then substantially uniformly stretched and aligned by a receding air/water interface. Schurra and Bensimon (2009) “Combing genomic DNA for structure and functional studies.” Methods Mol. Biol. 464: 71-90; See also U.S. Pat. No. [Bensimon, 1995, U.S. Pat. No. 7,122,647], both of which are herein incorporated by reference in their entirety.

The percentage of fully-stretched nucleic acid molecules depend on the length of the nucleic acid molecules and method used. Generally, the longer the nucleic acid molecules stretched on a surface, the easier it is to achieve a complete stretching. For example, according to [Conti, 2003], over 40% of a 10 kb DNA molecules could be routinely stretched with some conditions of capillary flow, while only 20% of a 4 kb molecules could be fully stretched using the same conditions. For shorter nucleic acid fragments, the stretching quality can be improved with the stronger flow induced by dropping coverslips onto the slides. However, this approach may shear longer nucleic acid fragments into shorter pieces and is therefore may not suitable for stretching longer molecules. See e.g., [Conti, 2003]Conti, et al. (2003) Current Protocols in Cytometry John Wiley & Sons, Inc. and [Gueroui, 2002] Gueroui, et al. (Apr. 30, 2002) “Observation by fluorescence microscopy of transcription on single combed DNA.” PNAS 99(9): 6005-6010, both of which are hereby incorporated by reference in their entirety. See also [Bensimon, 1994, U.S. Pat. No. 5,840,862], [Bensimon, 1995, WO 97/18326], [Bensimon, 1999, WO 00/73503], [Bensimon, 1995, U.S. Pat. No. 7,122,647] which are hereby incorporated by reference in their entirety. [Lebofsky, 2003] “Single DNA molecule analysis: applications of molecular combing.” Brief Funct. Genomic Proteomic 1: 385-96, hereby incorporated by reference in its entirety.

In some embodiments, the long nucleic acid molecule is attached to a substrate at one end and is stretched by various weak forces (e.g., electric force, surface tension, or optical force). In this embodiment, one end of the nucleic acid molecule is first anchored to a surface. For example, the molecule can be attached to a hydrophobic surface (e.g., modified glass) by adsorption. The anchored nucleic acid molecules can be stretched by a receding meniscus, evaporation, or by nitrogen gas flow. See e.g., [Chan, 2006] “A simple DNA stretching method for fluorescence imaging of single DNA molecules.” Nucleic Acids Research 34(17): e1-e6, herein incorporated by reference in its entirety.

In the general methods described herein where-by one end of the molecule is bound to a surface during stretching, the nucleic acids can be stretched by a factor of 1.5 times the crystallographic length of the nucleic acid. Without being bound by a particular theory, the ends of the nucleic acid molecule are believed to be frayed (e.g., open and exposing polar groups) that bind to ionizable groups coating a modified substrate (e.g., silanized glass plate) at a pH below the pKa of the ionizable groups (e.g., ensuring they are charged enough to interact with the ends of the nucleic acid molecule). The rest of the double-strand nucleic acid molecule cannot form these interactions. As the meniscus retracts, surface retention creates a force that acts on the nucleic acid molecule to retain it in the liquid phase; however this force is inferior to the strength of the nucleic acid molecule's attachment; the result is that the nucleic acid molecule is stretched as it enters the air phase; as the force acts in the locality of the air/liquid phase, it is invariant to different lengths or conformations of the nucleic acid molecule in solution, so the nucleic acid molecule of any length will be stretched the same as the meniscus retracts. As this stretching is constant along the length of a nucleic acid molecule, distance along the strand can be related to base content.

The pH of the solution used in a receding meniscus method can affect the efficiency of nucleic acid binding to the substrate. On hydrophobic surfaces good binding efficiency can be reached at a pH of approximately 5.5. For example, at pH 5.5, approximately 40-kbp DNA is 10 times more likely to bind by an extremity than by a midsegment. [Allemand, 1997] “pH-Dependent Specific binding and Combing of DNA.” Biophysical Journal 73: 2064-2070, herein incorporated by reference in its entirety.

In another embodiment, the nucleic acid molecule is stretched by dissolving the long nucleic acid molecules in a drop of buffer and running down the substrate. In a further embodiment, the long nucleic acid molecules are embedded in agarose, or other gel. The agarose comprising the nucleic acid is then melted and combed along the substrate.

In another embodiment, the nucleic acid molecule is combed on the surface by a receding meniscus, whereby the receding speed is controlled by a physical blade or mechanical fixture (herein collectively called “blade”) positioned above the surface onto which the molecule is to be combed, and said blade is moved relative to the surface of the combing surface, while maintaining a solution that comprises the meniscus pinned to the blade. In the preferred embodiment, the height of the blade and its speed relative to the combing surface are optimized for the combing application. In some embodiments, the blade's speed is more than 1 micron/second, or more than 10 microns/second, or more than 100 microns/second, or more than 1,000 microns/second. In some embodiments, the blade is in direct contact with the combing surface. In some embodiments, the blade is more than 1 micron above the combing surface, or more than 10 microns, or more than 100 microns, or more than 1,000 microns. In some embodiments, the height of the blade above the combing surface is maintained by a physical spacer. In some embodiments, the space is integrated in the blade. In some embodiments, the spacer is integrated in the substrate that comprises the combing surface.

In another embodiment, the nucleic acid molecule is combed on a transfer substrate, and then said transfer substrate is made contact with a target substrate, transferring the molecule. As an example, nucleic acid molecules are combed onto a PDMS substrate, which is then contacted with the target substrate, as previously demonstrated [Lee, PNAS, 2005].

In another embodiment, the molecule is attached to the substrate at least one specific point, allowing the remainder of the molecule a substantial amount of degree of freedom, such that portion of elongation in the molecule is obtained by the application of an an external force on the molecule in a direction that is substantially parallel to the surface of the substrate. Examples of such embodiments include “DNA curtains” [Gibb, 2012] where-by the point of attachment is a controlled process, or the point of attachment can be random via interactions of the molecule with fluidic features, for example pillars as shown by [Craighead, 2011, U.S. Pat. No. 9,926,552].

In some embodiments, molecular combing can be performed by elongating the molecules by flowing with an applied external force said molecules in a confining fluidic channel of an open fluidic device, such that after elongation in the device, the molecule is presented in an elongated state on the surface of the device, or within a porous film on the surface of the device. In the preferred embodiment, the applied external force is a fluid flow. In the most preferred embodiment, the fluid flow is driven by a capillary force. In one embodiment, the molecule is elongated via an elongation channel that can elongate the molecule via methods described elsewhere in this disclosure, including confining dimensions, external force, interaction with physical obstacles, interaction with a functionalized surface, or combination there-of. In some embodiments, the fluidic channels of the device not fully confined, such that after evaporation of the transporting solution, the molecules are at least partially immobilized on the surface of the device in an elongated state. In some embodiments, the cross section of fluidic channels of the device is of triangular tapered shape, with wider opening at the top and infinitely narrower bottom, substantially enclosed or not fully enclosed, such that after evaporation of the transporting solution, the suspended molecules are drawn down towards increasingly confining narrower bottom to be increasingly elongated, at least confined in a small volume of solution or partially immobilized on the surface of the device in a linearized state. In such embodiments with the cross section of fluidic channels of the device is of triangular tapered shape, with wider opening at the top and infinitely narrower bottom, the suspended molecules drawn down towards increasingly confining narrower bottom to be increasingly elongated and linearized, in ultra-confined small volume of solution or immobilized on the surface of the device, would be compatible with ultrahigh or super resolution imaging or interrogation. In some embodiments, as shown in in FIG. 2, a molecule 205 is elongated in a confined elongation channel of a microfluidic device (204), here with channel dimensions (202) that provide a confining environment and/or physical obstacles (203) that aid in promoting elongation. A gelling material within the solution that surrounds the molecule within the microfluidic device is then gelled. Finally, the molecules (215) are made accessible to the surface of the device via removal of the roof (201) while maintain the molecules within the gel film, or by using a porous roof material.

Fluidic Device. The term “microfluidic device” or “fluidic device” as used herein generally refers to a device configured for fluid transport and/or transport of bodies through a fluid, and having a fluidic channel in which fluid can flow with at least one minimum dimension of no greater than about 100 microns. The minimum dimension can be any of length, width, height, radius, or cross-sectional axis. A microfluidic device can also include a plurality of fluidic channels. The dimension(s) of a given fluidic channel of a microfluidic device may vary depending, for example, on the particular configuration of the channel and/or channels and other features also included in the device.

Microfluidic devices described herein can also include any additional components that can, for example, aid in regulating fluid flow, such as a fluid flow regulator (e.g., a pump, a source of pressure, etc.), features that aid in preventing clogging of fluidic channels (e.g., funnel features in channels; reservoirs positioned between channels, reservoirs that provide fluids to fluidic channels, etc.) and/or removing debris from fluid streams, such as, for example, filters. Moreover, microfluidic devices may be configured as a fluidic chip that includes one or more reservoirs that supply fluids to an arrangement of microfluidic channels and also includes one or more reservoirs that receive fluids that have passed through the microfluidic device. In addition, microfluidic devices may be constructed of any suitable material(s), including polymer species and glass, or channels and cavities formed by multi-phase immiscible medium encapsulation. Microfluidic devices can contain a number of microchannels, valves, pumps, reactor, mixers and other components for producing the droplets. Microfluidic devices may contain active and/or passive sensors, electronic and/or magnetic devices, integrated optics, or functionalized surfaces. The physical substrates that define the microfluidic device channels can be solid or flexible, permeable or impermeable, or combinations there-of that can change with location and/or time. Microfluidic devices may be composed of materials that are at least partially transparent to at least one wavelength of light, and/or at least partially opaque to at least one wavelength of light.

A microfluidic device can be fully independent with all the necessary functionality to operate on the desired sample contained within. The operation may be completely passive, such as with the use of capillary pressure to manipulate fluid flows [Juncker, 2002], or may contain an internally power supply such as a battery. Alternatively, the fluidic device may operate with the assistance of an external device that can provide any combination of power, voltage, electrical current, magnetic field, pressure, vacuum, light, heat, cooling, sensing, imaging, digital communications, encapsulation, environmental conditions, etc. The external device maybe a mobile device such as a smart phone, or a larger desk-top device.

The containment of the fluid within a channel can be by any means in which the fluid can be maintained within or on features defined within or on the fluidic device for a period of time. In most embodiments, the fluid is contained by the solid or semi-solid physical boundaries of the channel walls. FIG. 3 shows an example where-by channel walls with cross-sections such as rectangles (302), triangles (303), ovals (304), and mixed geometry (305) are all defined within a fluidic device (301). In other embodiments, fluidic containment within the fluidic device may be at least partially contained via solid physical features in combination with surface energy features [Casavant, 2013], or an immiscible fluid [Li, 2020]. Examples of a fluid being at least partially confined within physical boundaries include various channels physically defined on the surface of a fluidic device (306) such as grooves (307, 308) and rectangles (309, 310), all of which are filled with liquid of sufficiently minimal quantity, that surface tension allows for the liquid to be physically maintained within the channels, and not overflow. In other embodiments, the channel (311) could be a defined by a groove in a corner (312) of a fluidic device, or the channel (314) could be defined by two physically separated boundaries (313 and 315) of a fluidic device, or the channel (321) could be defined by a corner (320) of a fluidic device. In other embodiments, the channel (317) is defined by a hydrophilic section (318) on the surface of a fluidic device (316) where-by the hydrophilic section is bounded by hydrophobic sections (319) on the surface of the fluidic device. In all cases, these embodiments are non-limiting examples. Used herein, an “open fluidic device” is a fluidic device that comprises at least one fluidic channel in which the solution in said channel is at least partially exposed to a gas-phase interface. Examples include air, water vapor, oxygen, nitrogen, or mixtures thereof. In particular, with regards to the operation of an open fluidic device, the selection of the gas composition, pressure, and other environmental conditions may be controlled, and may be critical to the desired operation of the open fluidic device. For example, for a particular period of time, a particular temperature, or humidity, or dew point, or wavelength exposure may be desired.

In some embodiments, the fluidic device includes an “electrowetting device” or “droplet microactuator”, which is a type of microfluidic device capable of controlled droplet operations within the fluidic device via specific application of local electric fields. Non limiting examples of such devices include a liquid droplet surrounded by air on an open surface, and a liquid droplet surrounded by oil sandwiched between two surfaces. A detailed review of the various configurations of use, and physics of droplet control are provided by [Mugele, 2005] and [Zhao, 2013], both of which are provided here for reference.

It should be understood that some of the principles and design features described herein can be scaled to larger devices and systems including devices and systems employing channels and features reaching the millimeter or even centimeter scale channel cross-sections. Thus, when describing some devices and systems as “microfluidic,” it is intended that the description apply equally, in certain embodiments, to some larger scale devices. In addition, it should be understood that some of the principles and design features described herein can be scaled to smaller devices and systems including devices and systems employing channels and features that are 100s of nanometers, or even 10s of nanometers, or even single nanometers in scale channel cross-sections. Thus, when describing some devices and systems as “microfluidic,” it is intended that the description apply equally, in certain embodiments, to some smaller scale devices. As an example, a device may have input wells to accommodate liquid loading from a pipette that are millimeters in diameter, which are in fluidic connection with channels that are centimeters in length, 100s of microns wide, and 100s of nm deep, which are then in fluidic connection with nanopore constriction devices that are 0.1-10 nm in diameter.

A variety of materials and methods, according to certain aspects of the invention, can be used to form articles or components such as those described herein, e.g., channels such as microfluidic channels, chambers, etc. For example, various articles or components can be formed from solid materials, in which the channels can be formed via micromachining, film deposition processes such as spin coating and chemical vapor deposition, laser fabrication, photolithographic techniques, bonding techniques, deposition techniques, lamination techniques, molding techniques, etching methods including wet chemical or plasma processes, multi-phase immiscible medium encapsulation and the like. For patterning, a variety of methods may be employed, including but not limited to: photolithography, electron-beam lithography, nanoimprint lithography, AFM lithography, STM lithography, focused ion-beam lithography, stamping, embossing, molding, and dip pen lithography. For bonding, a variety of methods may be employed, including but not limited to: thermal bonding, adhesive bonding, surface activated bonding, fusion bonding, anodic bonding, plasma activated bonding, laser bonding, and ultra sonic bonding.

In one set of embodiments, various structures or components of the articles described herein can be formed of a polymer, for example, an elastomeric polymer such as polydimethylsiloxane (“PDMS”), polytetrafluoroethylene (“PTFE” or Teflon®), or the like. For instance, according to one embodiment, a microfluidic channel may be implemented by fabricating the fluidic system separately using PDMS or other soft lithography techniques [Xia, 1998, Whitesides, 2001].

Other examples of potentially suitable polymers include, but are not limited to, polyethylene terephthalate (PET), polyacrylate, polymethacrylate, polycarbonate, polystyrene, polyethylene, polypropylene, polyvinylchloride, cyclic olefin copolymer (COC), polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Combinations, copolymers, or blends involving polymers including those described above are also envisioned. The device may also be formed from composite materials, for example, a composite of a polymer and a semiconductor material. The device may be formed from glass, silicon, silicon nitride, silicon oxide, quartz, metal, fused silica, mica. The device may be formed from a combination of different materials that are mixed, bonded, laminated, layered, joined, deposited, evaporated, merged, or combination there-of.

Feature. Unless specifically stated otherwise, a “feature” is a region within or on the fluidic device defined by at least one boundary. In some embodiments, a boundary is defined by patterning. In some embodiments, a boundary may be a change in a physical topology, for example: a corner, a curve, an edge, a point, a depression, an inflection, a hill. Thus, for example, a feature may be channel, a wall, a pit, a hole, a pillar, a well, a floor, a roof. In some embodiments, a boundary may be a change in material composition or property, for example: a conductive material interfacing an insulating material, or a silicon nitride material interfacing with a silicon oxide material. Thus, a feature may be magnetic cube embedded in PMMA, or a polystyrene bead on glass surface. In some embodiments, a boundary may be change in a surface property, for example: a boundary may be a hydrophobic surface interfacing with a hydrophilic surface, or a non-functionalized surface interfacing with a functionalized surface. Thus, a feature may be a hydrophobic path on a hydrophilic COC surface, functionalized cell adhesion patterns among nonfunctionalized surface, or a circle functionalized with photo-cleavable barcodes on the surface of a silicon oxide substrate.

Physical Obstacle. Unless specifically stated otherwise, a “physical obstacle” is a physical feature within a fluidic device in which a long nucleic acid molecule, in the presence of an applied force, physically interacts with, such that the molecule's physical conformation or location is different than had said physical obstacle not been present. Non-limiting examples include: pillars, corners, pits, traps, barriers, walls, bumps, constrictions, expansions. The physical obstacles need not be physically continuous with the fluidic channel, but may also be additive to the device, with non-limiting examples including: beads, gels, particles.

Environmental Condition. An “environmental condition” may comprise any property of physics, matter, chemistry that surrounds a bio-molecule that may impact said bio-molecule's physical state, thermo-dynamic state, chemical state, or reactivity to other reagents. The impact on the bio-molecule may be due to the presence of the environmental condition, or a change in the environmental condition. An environmental condition may comprise a temperature, a pressure, a dew point, a humidity level, a pH, an ionic concentration, a flow rate or direction. An environmental condition may be flux, polarization, intensity of a wavelength of light. An environmental condition may comprise a solution composition, for example a concentration of a certain reagent within a solution, or a ratio of certain reagents within a solution, or a salt composition used for a particular buffer. An environmental condition may comprise an external force acting on a bio-molecule, for example a solution or air flow rate. An environmental condition may comprise thermal conductivity property, an electrical conductivity property, an optical opacity or transparency property. An environmental condition may be an electric or magnetic field. An environmental condition may be sound of a certain frequency or intensity. An environmental condition may be an ultrasonic wave of a certain frequency or intensity.

Reagent. Used herein, a “reagent” is any substance or compound added to a system to cause, enhance, attenuate, supply, or stop a chemical reaction, including an enzymatic reaction. A reagent may be a nucleotide, a nucleotide of a certain type (eg: A, T, C, G, U), a terminating nucleotide, a reversibly terminating nucleotide, an enzyme, a polymerase, a protein, a restriction enzyme, a nicking enzyme, a polynucleotide, an at least partially double-stranded polynucleotide, an at least partially single-stranded polynucleotide, an RNA, a guide RNA, a CRISPR-associated protein (CAS).

External Force. An “external force” or “external applied force” is any applied force on a body such that the force that can perturb the body from a state of rest or no acceleration (or deacceleration), or the removal of such a force can perturb the body from a state of rest or no acceleration (or deacceleration). Non-limiting examples include hydrodynamic drag exerted by a fluid flow [Larson, 1999] (which can be imitated by a pressure differential, gravity, capillary action, electro-osmotic), an electric field, electric-kinetic force, electrophoretic force, pulsed electrophoretic force, magnetic force, dielectric-force, centrifugal acceleration or combinations there-of. In addition, the external force may be applied indirectly, for example if bead is bound to the body, and then the bead is subjected to an external force such a magnetic field, or optical teasers.

Contact Probe. Used herein, a “contact probe” system is an instrument, or a component within an instrument that is capable of positioning the point or tip of a contact probe within the desired location in (x,y,z) space with respect to a surface, preferably with nanometer position accuracy or better, and measuring a signal as a function of the xy, or xyz position. In the preferred embodiments, the contact probe is capable of measuring a signal based on its interaction with a physical object. In the preferred embodiment, the contact probe comprises part of a contact probe interrogation system, which itself is a type of interrogation system. In the preferred embodiments, the contact probe is a surface scanning probe, capable of generating a signal while the probe is physically moved in xyz space with respect to the surface by the instrument. Different types of contact probes include SPM (Scanning Probe Microscopy), AFM (Atomic Force Microscopy), HS-AFM (High Speed Atomic Force Microscopy), STM (Scanning Tunneling Microscopy), SPE (Scanning Probe Electrochemistry), CFM (Chemical Force Microscopy), LFM (lateral Force Microscopy), magnetic force microscopy (MFM), high frequency MFM, magneto-resistive sensitivity mapping (MSM), electric force microscopy (EFM), scanning capacitance microscopy (SCM), Scanning spreading resistance microscopy (SSRM), tunneling AFM and conductive AFM, contact AFM, non-contact AFM, dynamic contact AFM, tapping AFM, kelvin probe force microscopy (KPFM), piezo-response force microscopy (PFM), photothermal microscpectroscopy, scanning gate microscopy (SGM), scanning quantum dot microscopy (SQDM), scanning voltage microscopy (SVM), force modulation microscopy (FMM), ballistic electron emission microscopy (BEEM), electrochemical scanning tunneling microscopy (ECSTM), scanning Hall probe microscopy (SHPM), spin polarized scanning tunneling microscopy (SPSM), photon scanning tunneling microscopy (PSTM), scanning tunneling potentiometry (STP), synchrotron x-ray scanning tunneling microscopy (SXSTM), Scanning Probe Electrochemistry (SPE), scanning electrochemical microscopy (SECM), scanning ion-conductance microscopy (SICM), scanning vibrating electrode technique (SVET), scanning Kelvin probe (SKP), fluidic force microscopy (FluidFM), feature-oriented scanning probe microscopy (FOSPM), magnetic resonance force microscopy (MRFM), near-field scanning optical microscopy NSOM, scanning near-field optical microscopy (SNOM), scanning SQUID microscopy (SSM), scanning spreading resistance microscopy (SSRM), scanning thermal microscopy (SThM), scanning single-electron transistor microscopy (SSET), scanning thermo-ionic microscopy (STIM), charge gradient microscopy (CGM), and scanning resistive probe microscopy (SRPM). For a review of different Scanning Probe Microscopy systems, refer to [Takahashi, 2017]. For clarity, a contact probe need not necessarily make intimate physical contact with the sample, or any object, to measure a signal from said sample.

Scanning tunneling microscopy was the first SPM technique developed in the early 1980's. STM relies on the existence of quantum mechanical electron tunneling between the probe tip and sample surface. The tip is sharpened to a single atom point and is raster scanned across the surface, maintaining a probe-surface gap distance of a few angstroms without actually contacting the surface. A small electrical voltage difference (on the order of millivolts to a few volts) is applied between the probe tip and sample and the tunneling current between tip and sample is determined. As the tip scans across the surfaces, differences in the electrical and topographic properties of the sample cause variations in the amount of tunneling current. In certain embodiments of the invention, the relative height of the tip may be controlled by piezoelectric elements with feed-back control, interfaced with a computer. The computer can monitor the current intensity in real time and move the tip up or down to maintain a relatively constant current. In different embodiments, the height of the tip and/or current intensity may be processed by the computer to develop an image of the scanned surface.

Because STM measures the electrical properties of the sample as well as the sample topography, it is capable of distinguishing between different types of conductive material, such as different types of metal in a metal barcode. STM is also capable of measuring local electron density. Because the tunneling conductance is proportional to the local density of states (DOS), STM can also be used to distinguish carbon nanotubes that vary in their electronic properties depending on the diameter and length of the nanotube. STM may be used to detect and/or identify any nano-barcodes that differ in their electrical properties.

In some embodiments where the contact probe comprises an AFM system, the system can operate in a variety of different modes, and thus measure a variety of different signals, depending on the selection of the probe type, its mode of operation, and the probe's tip sharpness. Non limiting examples of different AFM modes include non-contact mode, contact mode, tapping mode, dry mode, wet mode, high-frequency mode, ultra-high frequency mode, force-modulation mode, conductive mode, magnetic mode, super-sharp tip mode, diamond tip mode, high-aspect ratio mode, electron beam deposited tip mode, and carbon-nano-tube tip mode. In some embodiments, the contact probe can operate in a dry environment, or a humid environment, or a liquid environment. In some embodiments, the point of the contact probe can be functionalized with chemical moieties, biological bodies, or affinity groups to enable biochemical interaction with the physical object being probed. For a review of various functionalization that have been demonstrated on contact probes, refer to [Ebner, 2019]. In some embodiments, the point of the contact probe may include a carbon nanotube, a nanorod, or a nanospike. In some embodiments, the tip of the contact probe may include a pore, or nanopore, that allows for a fluidic connection to a fluidic channel or fluidic chamber within the contact probe.

In AFM microscopy, the probe is attached to a spring-loaded or flexible cantilever that is in contact with the surface to be analyzed. Contact is made within the molecular force range (i.e., within the range of interaction of Van der Waal forces). Within AFM, different modes of operation are possible, including contact mode, non-contact mode and TappingMode™.

In contact mode, the atomic force between probe tip and sample surface is measured by keeping the tip-sample distance constant and measuring the deflection of the cantilever, typically by reflecting a laser off the cantilever onto a position sensitive detector. Cantilever deflection results in a change in position of the reflected laser beam. As in STM, the height of the probe tip may be computer controlled using piezoelectric elements with feedback control. In some embodiments of the invention a relatively constant degree of deflection is maintained by raising or lowering the probe tip. Because the probe tip may be in actual (Van der Waal) contact with the sample, contact mode AFM tends to deform non-rigid samples. In non-contact mode, the tip is maintained between about 50 to 150 angstrom above the sample surface and the tip is oscillated. Van der Waals interactions between the tip and sample surface are reflected in changes in the phase, amplitude or frequency of tip oscillation. The resolution achieved in non-contact mode is relatively low.

In TappingMode™, the cantilever is oscillated at or near its resonant frequency using piezoelectric elements. The AFM tip periodically contacts (taps) the sample surface, at a frequency of about 50,000 to 500,000 cycles per second in air and a lower frequency in liquids. As the tip begins to contact the sample surface, the amplitude of the oscillation decreases. Changes in amplitude are used to determine topographic properties of the sample.

In this document, “scan” when used in association with a contact probe refers to the controlled movement of the contact probe in x, y, and z space while interrogating a sample, with respect to the physical position of the sample being interrogated. In some embodiments whereby the contact probe mode of operation comprises the contact probe to vibrate, the scan may comprise the controlled movement of the time averaged position of the tip over some sampling time period in x, y, z space while interrogating a sample, with respect to the physical position of the sample being interrogated. In some embodiments, a scan may comprise moving the contract probe along a path in 3D space. In some embodiments, a scan may comprise moving the contract probe along a path within a 2D plane. In some embodiments, a scan may comprise moving the contract probe along a path within a 1D line. In some embodiments, a scan may comprise a constant velocity movement. In some embodiments, a scan may comprise a velocity that varies or changes with time. In some embodiments, a scan may comprise a series of stops and starts. In some embodiments, a scan may comprise moments where the probe is motionless during interrogation, or the average velocity of the contact probe over some sampling time period in x, y, z space is zero. In some embodiments, a scan may comprise moving the contact probe tip in a circular fashion within a 2D plane, or an oval fashion, or rectangular fashion, or a closed path fashion.

A fundamental limitation for all contact probe interrogation systems in the inherent serial nature of data collection, as such, when the contact probe is operating, there typically is a trade-off with respect to scanning speed, spatial resolution, and measurement noise of the signal being measured. For example: a single scan of length L, consisting of a movement along a path in the xy plane, will be traversed in time T, and assuming a constant velocity, collect P pixels of data, each representing a length segment L/P, and each requiring T/P amount of time to collect. In order to achieve high spatial resolution (small L/P) and low-noise measurement for each pixel, L must be reduced, or T must be increased, or both, until the mechanical and sensor limitations with respect to the contact probe interrogation system are encountered. As such, it's highly advantageous to position the contact probe tip only in the regions of interest, so as to efficiently use time.

In some embodiments, a contact probe interrogation system comprises multiple contact probes. In some embodiments, the collection of contact probes are all of the same type. In some embodiments, at least one contact probe within the set of contact probes is different. In some embodiments, the contact probes can all act independently with respect to their movement and orientation of their respective tips with their respective scanning surface. In some embodiments, at least two contact probes share at least one shared movement and orientation of their respective tips with respect to the scanning surface. For example: two contact probes may have independent z control, but share the same stage xy and rotation.

Computer Based system. A “computer-based system” or “computer program” refers to the hardware means, software means, and data storage means used to analyze information. The minimum hardware of a subject computer-based system comprises a central processing unit (CPU), input means, output means, data storage means, access to the Internet and data available therein. A skilled artisan can readily appreciate that any one of the currently available computer-based system are suitable for use in the present invention. The data storage means may comprise any manufacture comprising a recording of the present information as described above, or a memory access means that can access such a manufacture.

### BRIEF DESCRIPTION OF DRAWINGS

For all drawings, the use of roman numerals: i), ii), iii), iv), etc are to denote a passage of time. Unless specifically stated, the figures are not drawn to scale.

FIG. 1(A) demonstrates an embodiment of generating a linear physical map along the length of a long nucleic acid molecule by cleaving the molecule at known recognition sites producing an ordered pattern of lengths.

FIG. 1(B) demonstrates an embodiment of generating a linear physical map by attaching label bodies at known recognition sites producing an ordered pattern of segments.

FIG. 1(C) demonstrates an embodiment of generating a linear physical map by attaching label bodies along the length of molecule in a manner such the density of the labelling bodies correlates with the underlying AT/CG ratio.

FIG. 2 demonstrates an enclosed fluidic device and method for generating combed linearly elongated nucleic acid molecule in parallel fashion, with (i) showing the molecules being flown into an enclosed channel, and with (ii) showing said molecules after the roof is removed from the channel.

FIG. 3 demonstrates different, non-limiting embodiments of confined and non-confined channel types within a fluidic device.

FIG. 4 demonstrates an embodiment whereby (i) a population of long nucleic acid molecules elongated on a substrate or open fluidic device have their respective physical maps optically interrogated, and then (ii) said physical maps are aligned to a reference to identify at least one ROI, and then (iii) a contact probe is directed to the physical location of the ROI to further interrogate the ROI.

FIG. 5 demonstrates an embodiment whereby a contact probe is directed to an ROI of a long nucleic acid molecule elongated on a substrate or open fluidic device, where said ROI is determined from an analysis of the molecule's physical map, and the ROI is located on a portion of the molecule that is suspended.

FIG. 6(A) demonstrates an embodiment whereby a contact probe is directed to an ROI of a long nucleic acid molecule elongated on a substrate or open fluidic device to interrogate the topological profile of the molecule with its bound labeling bodies within the ROI.

FIG. 6(B) demonstrates an embodiment whereby a contact probe is directed to an ROI of a long nucleic acid molecule elongated on a substrate or open fluidic device to interrogate the topological profile of the molecule with its bound labeling body or higher order structure within the ROI.

FIG. 6(C) demonstrates an embodiment whereby a contact probe is directed to an ROI of a long nucleic acid molecule elongated on a substrate or open fluidic device to interrogate the molecule ROI, where the ROI comprises a single-strand nucleic acid, and the contact probe measures a signal as it scans along the length of the molecule.

FIG. 7(A) demonstrates an embodiment whereby a contact probe is directed to an ROI of a long nucleic acid molecule elongated on a substrate or open fluidic device to interrogate the conductivity profile of the molecule with its bound labeling bodies within the ROI.

FIG. 7(B) demonstrates embodiment whereby a contact probe is directed to an ROI of a long nucleic acid molecule elongated on a substrate or open fluidic device to interrogate the molecule ROI, where the ROI comprises a single-strand nucleic acid, and the contact probe measures an electrical signal as it scans along the length of the molecule.

FIG. 8 demonstrates an embodiment where-by the contact probe is used to generate a topological physical map that is of a much higher resolution than the fluorescent physical map within the ROI.

FIG. 9(A) demonstrates an embodiment of a patterned open fluidic device to be used for combing on to the surface long nucleic acid molecules.

FIG. 9(B) demonstrates an embodiment of combing long nucleic acid molecules onto the surface of a patterned open fluidic device by means of a blade.

FIG. 10 demonstrates an embodiment of identifying an ROI consisting of an insertion within a physical map generated by optical interrogation of a long nucleic acid molecule by alignment of said map to a reference, and then further interrogating said ROI with a contact probe to resolve the presence of a nucleic acid segment that is repeated 7 times.

FIG. 11(A) demonstrates an embodiment whereby the positional relationship of certain genomic information within a physical map of an immobilized long nucleic acid molecule on the surface of a substrate or open fluidic device is used at least in part to identify an ROI.

FIG. 11(B) demonstrates an embodiment whereby the positional relationship of certain genomic information within a physical map of an immobilized long nucleic acid molecule on the surface of a substrate or open fluidic device is used at least in part to identify an ROI.

FIG. 11(C) demonstrates an embodiment whereby the positional relationship of certain genomic information within a physical map of an immobilized long nucleic acid molecule on the surface of a substrate or open fluidic device is used at least in part to identify an ROI.

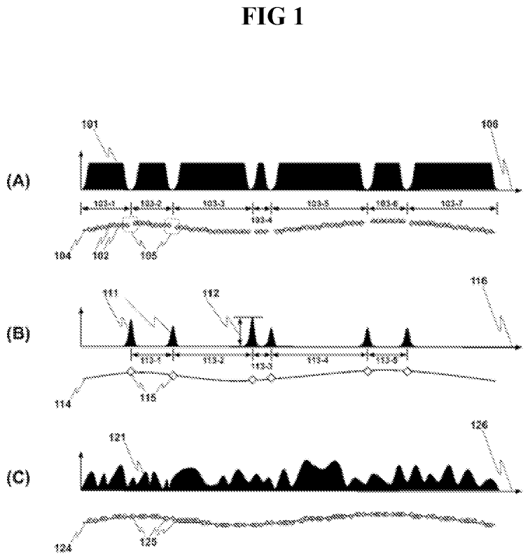

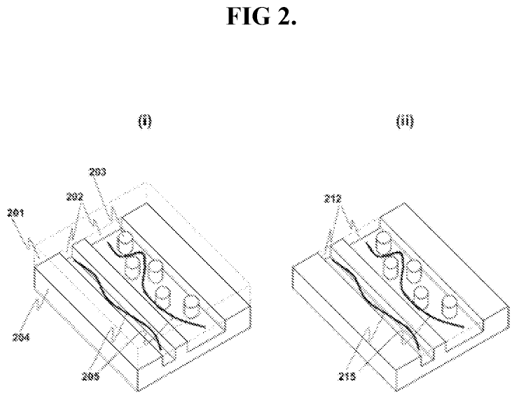

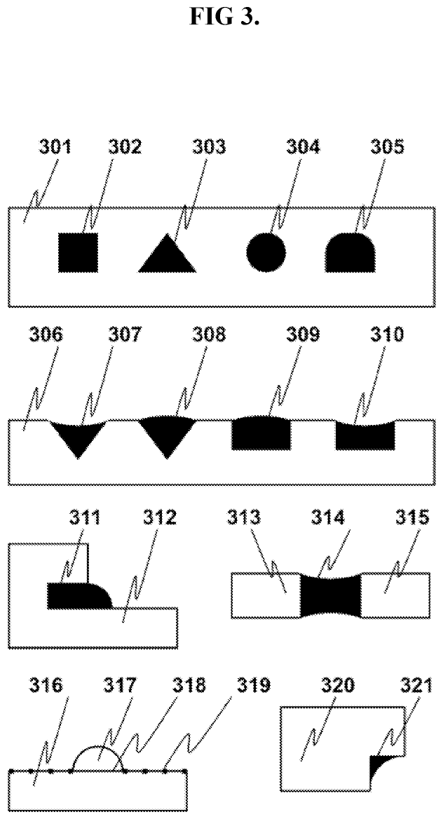

### DETAILED DESCRIPTIONS

Disclosed here are methods and devices for interrogating at least one region of interest (ROI) within at least one long nucleic acid molecule from a sample. The methods generally involve at least two modified long nucleic acid molecules on an substrate or open fluidic device in a substantially elongated configuration, where the degree of modification within the molecules generates a physical map within each molecule of sufficient variation to distinguish between said molecules, and where said physical maps can be optically interrogated and then compared or aligned to a reference, the resulting output of which is at least partially used to identify at least one ROI within at least one molecule, and then registering said at least one ROI's physical coordinates with respect to the underlying substrate or open fluidic device; and then further interrogating the at least one ROI by directing a contact probe to scan within the at least one ROI's registered coordinates to measure a signal. The present invention further provides a computer program and interrogation system product for use in a subject method.

In some embodiments, the optical interrogation of the long nucleic acid molecule comprises fluorescent imaging. For example, the fluorescent imaging of a fluorescent physical map along an elongated molecule, where in some embodiments, said physical map is comprising a plurality of bound intercalating dyes varying in density per base-pair in correlation with the underlying AT-CG content to form a melt-map. In some embodiments, the optical interrogation of the long nucleic acid molecule comprises brightfield imaging. For example, the bright field imaging of a physical map within a metaphase chromosome, where in some embodiments, said physical map is comprising a plurality of bound stain dye molecules that vary in density within the long nucleic acid molecule in correlation with the local AT-CG content to form a karyotype banding pattern.

In some embodiments, the targeted interrogation by a contact probe of the ROI allows for the generation of genomic information within the ROI. In some embodiments, the genomic information comprises sequence information. In some embodiments, the genomic information comprises a new physical map. In some embodiments, the genomic information comprises a higher-resolution version of the physical map that was previously generated by optical interrogation. In some embodiments, the genomic information comprises the presence, change, or lack of a structural variation, sequence, or higher-order nucleic acid structure.

In some embodiments, at least one additional ROI may be interrogated by the contact probe within a molecule based at least partially on the analysis of an ROI previously interrogated on said molecule or a different molecule by the contact probe. In some embodiments, the analysis may include data generated from the optical physical maps.

The sample containing long nucleic acid molecules will in many embodiments include more than one long nucleic acid molecule. The long nucleic acid molecules that are presented on the surface of a substrate or open fluidic device can include more than 1 distinct species, or more than 10 distinct species, or more than 100 distinct species, or more than 10,000 distinct species, or more than 100,000 distinct species, or more than 1,000,000 distinct species. The term “different species” refers to long nucleic acid molecules that differ from one another in nucleotide sequence by at least one nucleotide.

In some embodiments, the long nucleic acid molecule is immobilized during optical interrogation. In some embodiments, the long nucleic acid molecule is immobilized during interrogation by the contact probe.

In embodiments whereby the long nucleic acid molecule is immobilized prior to optical or contact probe interrogation, the molecule may be modified to generate a physical map before or after immobilization.

In some embodiments, a subject computer program will store one or more of the following information: 1) the physical location(s) of an immobilized long nucleic acid molecule on a substrate or open fluidic device; 2) an in-silco representation of said molecule's physical map generated by optical interrogation, with said physical map mappable along or within said molecule in base-pair space, or with said physical map mappable to the physical coordinates along or within said molecule on the substrate or open fluidic devices in physical position space; and 3) any ROI(s) along with their respective coordinates with respect to the originating molecule or underlying substrate or open fluidic device determined at least in part by an analysis of the physical map aligned to at least one reference; and 4) the genomic information obtained by a scan of the ROI(s) by the contact probe.

In some embodiments, the process for determination of the ROI(s) within a long nucleic acid molecule may include additional information from features obtained with the optical interrogation of said molecule. In some embodiments, the additional feature may include the identification of higher order structures in the molecule. In some embodiments, the additional feature may include the identification of knots, folds, loops, or spirals in the molecule. In some embodiments, the additional feature may include the identification of long nucleic acid molecule being a circular molecule, or having originated from a circular molecule. In some embodiments, the additional feature may include the identification of at least one labelling body bound to the molecule. In some embodiments, the additional feature may include the identification of at least one protein bound to the molecule. In some embodiments, the additional feature may include the identification of variation in the molecule's stretch or density per unit length or area on the substrate or open fluidic device. As an example, an ROI may be selected based on the observation of a loop structure in the long nucleic acid molecule that is in proximity of a gene that is identified by an analysis of the physical map. In another example, an ROI may be selected within a certain region of the physical map of a long nucleic acid molecule coupled with the observation that the molecule is circular in topology. In another example, and ROI may be selected based on the observation of a bound protein in the long nucleic acid molecule that is in proximity of a transcription factor that is identified by an analysis of the physical map.

In some embodiments, the substrate or open fluidic device will include fiducials, or markers, or physical registration points that allow for the interrogation system to obtain a repeatable x-y coordinate grid of the surface of the substrate or open fluidic device.

In some embodiments, the surface of the substrate or open fluidic device onto which the long nucleic acid molecules are immobilized retains nucleic acids. In some embodiments, the surface comprises a nucleic acid protection layer adsorbed onto the surface, which layer protects the immobilized nucleic acids from degradation. In some embodiments, the nucleic acid protection layer includes one or more agents that inhibit nucleic acid degradation. For example, in some embodiments, the nucleic acid protection layer includes one or more nuclease inhibitors. RNase inhibitors include, e.g., diethylpyrocarbonate.

In some embodiments, the surface of the substrate or open fluidic device onto which the nucleic acids are immobilized allows for one or more modification steps and/or other steps (e.g., washing), while maintaining the capacity to retain the long nucleic acid molecules. The surface of the insoluble support onto which the nucleic acids are immobilized also allows for one or more drying steps. The surface of the substrate or open fluidic device onto which the nucleic acids are immobilized does not exhibit any undesired chemical or electronic interaction with a contract probe.

In some embodiments, the surface of the substrate or open fluidic device onto which long nucleic acid molecules are immobilized is chemically modified to retain nucleic acids. Chemical modification of the surface is generally carried out by reacting the surface with a linking agent. A suitable linking agent comprises a moiety that binds to the surface (a surface binding moiety); and a moiety that binds to the nucleic acid (a nucleic acid binding moiety). In some embodiments the linking agent can be selectively cleaved or broken, allowing for separation of the long nucleic acid molecule from the surface. In some embodiments the cleavage is photo-cleavage. In some embodiments the cleavage is chemical cleavage. In some embodiments, the cleavage is done selectively based on some selection criterion.

In some embodiments, a linking agent is a silane compound, e.g., an organosilane such as a glycidoxypropyltrimethoxysilane or an aminopropyltriethoxysilane. In some embodiments, a linking agent comprises a silane moiety that binds to a surface; and an organic moiety that binds to a nucleic acid (e.g., covalently or non-covalently binds nucleic acid). An organic moiety that binds to a nucleic acid will in some embodiments comprise an amino group or a primary amine. Suitable silane compounds include, but are not limited to, epoxy-silane, 3-aminopropyl triethoxysilane (APTES), 3-glycidoxypropyltrimethoxy silane, vinyl silane, chlorosilane, and the like.

In some embodiments, nucleic acids are immobilized onto the surface by charge, e.g., the surface of the insoluble support is derivatized such that it has a net positive charge. In some embodiments, the surface is derivatized using APTES.

In one preferred embodiment of the open fluidic device, the input sample solution and any associated reagent solutions required to operate the device, can be loaded via manual pipette dispensing or automated liquid handling systems. In one preferred embodiment of the open fluidic device, the operation of the device may be controlled by at least one control instrument, which in turn, may be controlled by a program, computer based system, or a person(s). Operation of the device by the control instrument can include manipulating the physical position and conformation of the long nucleic acid molecule via the application of external forces, exposing the molecule to various reagent compositions and concentrations for various time periods and temperatures, optically interrogating the molecule to facilitate analysis of its composition or physical map as part of a feedback system to control the operation of the device, or extracting desired molecules or portions of molecules from the device. The open fluidic device and control instrument can interface in a number of ways. A non-exhaustive list includes: fluidic ports (both open and sealed), electrical terminals, optical windows, mechanical pads, heat pipes or sinks, inductance coils. A non-exhaustive list of potential functions the control instrument may perform on the device include: temperature monitoring, applying heat, removing heat, modifying an environmental conditions, measuring an environmental condition, applying pressure or vacuum to ports, measuring vacuum, measuring pressure, applying a voltage, measuring a voltage, applying a current, measuring a current, applying electrical power, measuring electrical power, exposing the device to focused and/or unfocused light, collecting the light generated or reflected from the device.

In one embodiment, the operation of the optical interrogation of the long nucleic acid molecule on the substrate or open fluidic device is controlled by a control instrument. In one embodiment, the operation of the contact probe interrogation of the long nucleic acid molecule's ROI(s) on the substrate or open fluidic device is controlled by a control instrument. The control instrument may be centrally located, or have different parts distributed for different or redundant functions.

In order to run the operation software on the control instrument, and perform collection and analysis of the data generated via interrogation, optically or with a contact probe, a non-exhaustive list of potential options include: localized processing within the control instrument, adjacent processing via a direct communication connection, external processing via a network connection, or combination there-of Various examples of processing modules include: a PC, a micro-controller, an application specific integrated micro-chip (ASIC), a field-programmable gate array (FPGA), a CPU, a GPU, a network server, cloud computing service, or combinations there-of.

In some embodiments, the control instrument may include an imaging system capable of optical interrogation, which may include any of the following types of imaging, or combinations there-of: fluorescent, epi-florescent, total internal reflection fluorescence, dark field, bright field, confocal.

In some embodiments, the control instrument may be able to fire multiple light sources simultaneously, or in series, and be able to image multiple colors simultaneously, or in series. If imaging multiple colors simultaneously, this may be done on different cameras, on a single camera but different regions of the sensor array, or on the same sensor of the same camera. In some embodiments, the wavelength of light fired by the control instrument is chosen so as to interact with the sample, the sample labeling body, or a functionalized surface in some way. Non limiting examples include: photo-cleaving of the nucleic acid, photo-cleaving photo-cleavable linkers, manipulating optical tweezers, activating photo-activated reactions.

In some embodiments, the control instrument may have at least one photosensitive sensor, of which non-limiting examples include: CMOS camera, SCMOS camera, CCD camera, photomultiplier tube (PMT), Time Delay & Integration (TDI) sensor, photodiode, light dependent resistor, photoconductive cell, photo-junction device, photo-voltaic cell.

In some embodiments, the control instrument may have at least one xy-stage or xyz stage, allowing for the imaging system to image different regions of the device, or other devices in the control instrument.

In some embodiments, the control instrument may have 1 or more motors or actuators capable of adjusting the device's interrogation region relative the control instrument's optical path, including rotation, z, tip, and tilt, based on an auto-focus feedback system, software analysis of image quality, device accessibility requirements, user access, or combination there-of.

The control instrument may be capable of robotic transport of one or more devices to different parts of the control instrument.

In some embodiments the substrate or open fluidic device can include fiducial markers or alignment markers that can be used to enable visual alignment of the substrate or device either manually or with the control instrument's program. In some embodiments, there are multiple zones on the substrate or open fluidic device, with each zone designed to physically isolate different input samples. In some embodiments, there are fiducial markers on the substrate or device that guide the user or automated dispensing system where on the device to dispense solution.

In the preferred embodiment, the control system comprises a contact probe interrogation system capable of positioning the tip in xyz space relative to physical coordinate system defined on the surface of the substrate or open fluidic device by the control system.

In some embodiments, the substrate forms part of a fluidic device. In the preferred embodiment, the fluidic device is an open fluidic device. In some embodiments, the open fluidic device comprises fluidic channels that allow for the flow via an external force of at least one long nucleic acid molecule within the channel. In some embodiments, at least a portion of the long nucleic acid molecule can be maintained in an elongated state within the fluid confines of the fluidic channel during optical interrogation of the molecule's physical map. In some embodiments the channel has a confining dimension of 10 microns or less, or 5 microns or less, or 2 microns or less, or 1 micron or less, or 500 nm or less, or 200 nm or less, or 100 nm or less, or 50 nm or less. In some preferred embodiments, the external force applied on the long nucleic acid molecule in the open fluidic channel is electro-kinetic in nature. In some preferred embodiments, the external applied force is a fluid flow, with said flow driven at least in part by capillary forces.

In some embodiments where-by long nucleic molecules can be transported in a fluidic channel of an open fluidic device, the long nucleic acid molecule's physical map can be interrogated by flowing the molecule into the detection region for optical interrogation. In some embodiments where-by the long nucleic acid molecules are immobilized on the surface of a substrate or open fluidic device, the immobilized molecules can be interrogated by physically moving the substrate or open fluidic device relative to the detection region for optical interrogation.

In some embodiments where-by the long nucleic acid molecule is transported through a fluidic channel of an open fluidic device, the contact probe may interrogate the molecule's ROI while said ROI is contained within said fluidic channel's solution, with the contact probe entering the solution via the open portion of the channel. In some embodiments, the contact probe interrogates the molecule's ROI after at least the solution containing said ROI is evaporated, allowing for said ROI to be immobilized on the surface of the confining physical features of the open channel. In some embodiments, a solution may be re-introduced to the channel, allowing for re-suspension of the molecule within the channel, and subsequent additional transport of the molecule in the channel via the application of an external force.

In some embodiments, the long nucleic acid molecules are combed onto a substrate or open fluidic device. In the preferred embodiment, the molecules are combed on the surface of the open fluidic device via a blade that controls the speed and angle of the meniscus, as said meniscus combs the molecules onto the surface of the open fluidic device. In the preferred embodiment, the open fluidic device comprises a collection of substantially hydrophilic channels separated from each other by a surface that is substantially hydrophobic. In some embodiments, the channels have a surface that is lower than the surface that separates adjacent channels. In some embodiments, this depth is less than 50 microns, or less than 20 microns, or less than 10 microns, or less than 1 micron, or less than 0.5 micron, or less than 0.2 micron, or less than 0.1 micron, or less than 0.05 micron,

In the preferred embodiments where the long nucleic acid molecule is at least partially immobilized on the surface of a substrate or open fluidic device, the surface has a surface roughness of less than 2 nm rms, or less than 1 nm rms, or less than 0.5 nm rms, or less than 0.2 nm rms. In some embodiments the surface may comprise silicon, or glass, or quartz, or fused silica, or mica, or a semiconductor.

In some embodiments the long nucleic acid molecules that are immobilized on the surface of the substrate all originate from a single cell, or collection of specific cells. In some embodiments, the collection of specific cells originates from a tissue sample, or biopsy. In some embodiments, the originating cell(s) are selected by a selection criterion and a sorting device. In some embodiments, the long nucleic acid molecules may originate from a random collection of cells.

In some embodiments, the long nucleic acid molecules that are immobilized on the surface of the substrate comprise both chromosomal and non-chromosomal long nucleic acid molecules that derive from a single cell. In some embodiments, the non-chromosomal long nucleic acid molecule is a circular DNA, in particular an ecDNA. In some embodiments, the non-chromosomal long nucleic acid molecule originates from the cell's cytosol. In some embodiments, the non-chromosomal long nucleic acid molecule is micronuclei derived

In some embodiments, the ROI may be selected at least in part from an analysis of the alignment of at least one molecule's physical map to at least one reference, with selection criteria that can change with time, including user preferences, the family health history of the originating sample's organism, the symptoms of the originating sample's organism, data from a clinical or biological or molecular test associated with the originating sample's organism. The ROI may be a gene, a structural variation (SV), a methylation pattern, a labelling body, a portion of a physical map, a sequence, a portion of a sequence, a higher order nucleic acid structure. The ROI may be an unidentified region within the physical map, or a region that may have an association with another ROI, directly or indirectly. The ROI may be a regulatory region, or a transcription factor binding site. The ROI may be associated with at least one disease. The ROI may be associated with risk-factors for development or onset of at least one disease. The ROI may be a chromosomal region, a chromatin section, a compaction feature, an interaction or binding site, a regulatory factor or complex, a binding site, a transcription factor binding site, a TAD, a CRISPR binding site or complex, an SV, a phasing block, a regulatory or modification enzyme binding site, a restriction enzymes sequence motif, a methylation binding body, a centromeric region, a sub-telomeric region, a portion of telomere, a mobile element, a repetitive element, a viral insertion site. The ROI may comprise at least a portion of a higher order structure. The ROI may comprise at least one labelling body that is bound to the long nucleic acid molecule, or a bound to a body that is bound to the long nucleic acid molecule. The ROI may comprise a region within the long nucleic acid molecule where the desired genomic information is unclear or only partially known from the optical interrogation of the molecule's physical map, and for which a higher resolution interrogation with a contact probe is desired. For example, analysis of the physical map may suggest the presence of a series of repeats flanked between two known regions identified by comparison or alignment to a reference, however the repeated sequence is too small to allow for a precise count of the repeats to be determined by an analysis of the physical map generated by optical interrogation. In this example, targeted inspection of the repeat region by the contact probe can be used to elucidate a more accurate assessment of the number of repeats. The ROI may comprise a component for which there is a temporal or dynamic aspect that may change the nature of the ROI, for example a cohesin loop that is in the process of being extruded.

The ROI may be selected based on the positional relationship of various genomic information within the physical map with respect to each other. For example, an ROI may be selected based on the order in which certain genes are located with respect to each other within the physical map. The ROI may be selected based on the positional relation of a regulatory region and a gene with respect to each other in the physical map. The ROI may be selected based on the positional relationship of a various genomic information within the physical with respect to a labeling body or a higher order nucleic acid structure. For example, an ROI may be selected based on the physical proximity of a gene to a knot, or the physical proximity of a gene to a labeling body specifically bound to a promoter region.

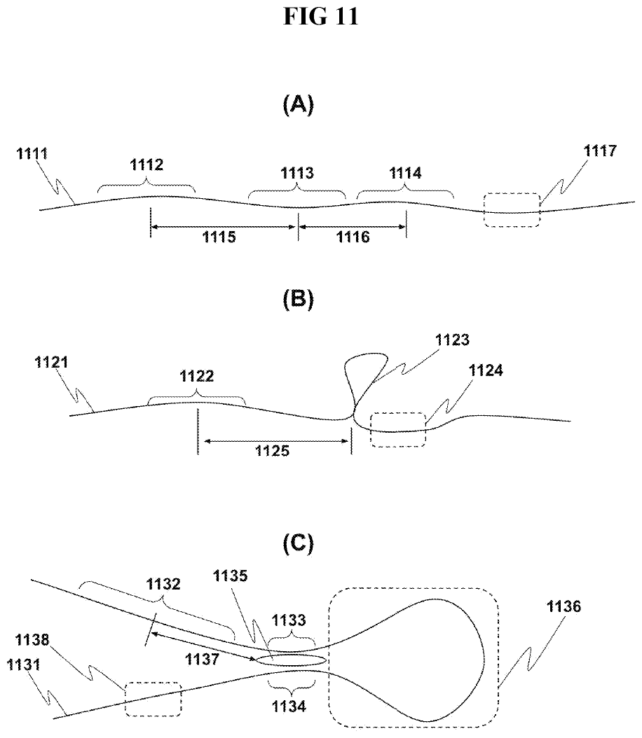

FIGS. 11(A), 11(B), and 11(B) demonstrates some embodiments whereby the positional relationship of certain genomic information within a physical map of an immobilized long nucleic acid molecule on the surface of a substrate or open fluidic device is used at least in part to identify an ROI. In FIG. 11(A), an analysis of a physical map of a long nucleic acid molecule (1111) aligned to a reference is used to identify the physical location of three separate genes within the molecule: (1112, 1113, and 1114). In this particular embodiment, the relative positional order of these genes within in the molecule, along with their physical distances from each other (1115, 1116) is used to identify a ROI within the molecule (1117). In FIG. 11(B), an analysis of a physical map of a long nucleic acid molecule (1121) aligned to a reference is used to identify a gene (1122). Furthermore, an identification of a higher order nucleic acid structure within the molecule by optical interrogation, here a loop (1123), that is within a certain distance (1125) of the identified gene, together with the gene comprises a Landmark. The location of the ROI (1124) is computed from the relative location of the loop (1123), prior knowledge of the offset from the loop to the ROI from previous studies of the region and the experimentally derived DNA stretch ratio from the distance between the gene (1122) and the loop (1123), together with the direction of the vector between the gene (1122) and the loop (1123). In FIG. 11(C), an analysis of a physical of long nucleic acid molecule (1131) aligned to a reference is used to identify a gene (1132). Furthermore a loop (1136) in the molecule that is maintained by a at least one protein (1135) is identified with optical interrogation, and an analysis of the physical proximity (1137) between the at least one protein and the gene is used to identify the at least one protein as a transcription factor complex associated with the identified gene (1132), with the associated promoter (1133) and enhancer (1134) regions. From this analysis, an ROI is selected (1138).

The ROI may be selected at least in part by some computer algorithm, or patient diagnosis, or disease hypothesis, or experimental hypothesis. The ROI may be selected by the user on-the-fly, or selected based on observations and analysis of other ROIs. The ROI may be selected at least in part based on the analysis or alignment of physical maps of other long nucleic acid molecules.

In some embodiments all identified ROI(s) are targeted. Alternately, not every or any ROI need be targeted. In some embodiments, ROI(s) are identified such that they inform the identification of additional ROI(s). In some embodiments, only a subset of ROI(s) are targeted. In some embodiments, a subset of ROI(s) from a first subset of molecules are used to identify an additional a subset of additional ROI(s) in a second subset of molecules. The first and second subsets of molecules can both each have an occupancy of at least one molecule, and the union of the first and second subsets can be zero or more molecules.

The ROI may be a single region along the length of a molecule such as a long nucleic acid molecule, or multiple regions. The ROI(s) may be each selected from separate criterion, or a combination of criterion. For example, one ROI on a long nucleic acid molecule may represent one gene, and a second ROI on the same molecule may represent a different gene. Optionally, a plurality of ROI(s) may represent a single higher-level ROI, for example, a series of ROI(s) that are all copies of the same genomic material, but located in different locations within a molecule such as a long nucleic acid molecule. An ROI may be defined as the boundary, neighbor, brake-point, or flanking region of another ROI. The ROI(s) may be continuous along the molecule, discontinuous, or combination there-of. An ROI(s) may be defined in the negative, for example the non-ROI region(s). The ROI may constitute the long nucleic molecule in its entirety, or a majority there-of, or a portion down to a small portion of a molecule such as a nucleic acid molecule. In some embodiments, there may be at least 1, 2, 3, 5, 10, 25, 100, 500, 1000, 10000, 100000 or more ROI(s) within a long nucleic acid molecule. For embodiments where-by a long nucleic acid molecule constitutes a chromosome, or a large portion of a chromosome, ROI(s) could be all, or a subset-of-all, genes along the molecule, or all, or a subset-of-all, transcription factor binding sites, or all, or a subset-of-all regulatory regions. Other ROIs are also consistent with the disclosure herein.

Such described embodiments are advantageous as contact probe interrogation systems are a relatively slow form of interrogation when compared to imaging, however they offer the ability to interrogate at a much higher resolution, including single base pair, and sub-single base-pair resolution. For example, scanning an area of 100 microns×100 microns with a contact probe interrogation device requires minutes to hours, depending on the desired spatial resolution and noise level of the scan, whereas a fluorescent interrogation of a similar sized area can be completed in milliseconds. Thus, first optically interrogating a long nucleic acid molecules on the surface of an open fluidic device or substrate to generate a physical map with an associated set of physical coordinates of ROI boundaries, regions, areas, or paths for further interrogation via a contract probe device is advantageous in that the contact probe can be controlled to scan any arbitrary region, thus the contact probe's scanning parameters (speed, scan paths, scan direction, pressure, force, frequency, pitch, period, direction, iterations, vibrating frequency, tunneling current, tip diameter, tip sharpness, tip material, etc) can be individually selected for a particular ROI, or region of the ROI, and further optimized for the desired resolution and data acquisition speed.

In the preferred embodiment, optical images of the surface of the substrate or open fluidic device are captured at a rate of more than 100 microns squared per second, or more than 1,000 microns squared per second, or more than 10,000 microns squared per second, or more than 100,000 microns squared per second, or more than 1,000,000 microns squared per second, or more than 10,000,000 microns squared per second. In some embodiments, adjacent images of the surface are stitched together to form a single optical image for analysis.

In the preferred embodiment, the physical maps of more than 1 long nucleic acid molecule can be optically interrogated per second, or more than 10 long nucleic acid molecule can be optically interrogated per second, or more than 100 long nucleic acid molecule can be optically interrogated per second, or more than 1,000 long nucleic acid molecule can be optically interrogated per second, or more than 10,000 long nucleic acid molecule can be optically interrogated per second.

In some embodiments, the present invention provides a computer program product for measuring the length of an immobilized nucleic acid and/or carrying out the conversion from length in physical coordinates (eg: nm, microns) of a long nucleic acid molecule as determined by contact probe interrogation to length in base pairs. The present invention thus provides a computer program product including a computer readable storage medium having a computer program stored on it.

Typical contact probe interrogation of a target involves scanning or rastering a probe tip across a surface line by line to record a series of information profiles as a function of x-y position on the surface that are then combined to form a representation of the ROI properties being measured, with examples of information profiles including: height (z), error, conductivity, current, charge, phase, magnetic field. The raster process takes considerable time as it is inherently serial in its operation, and dictated by the scan speed, the scan length and the number of lines recorded in the image.

The present invention provides a computer program product comprising a fast acquisition data analysis algorithm that provides for substantially improved efficiency in data collection with a contact probe interrogation system, by limiting the scanning time of the contact probe to interrogating only ROI(s), by using a parallel high-through optical interrogation process to identify the ROI(s) and their associated physical coordinates on the substrate or open fluidic device.

In some embodiments, a subject computer program product comprises an algorithm that provides for acquiring 2 or more cross-sectional profile data points at a given lateral position along a ROI. In some embodiments, a subject computer program product comprises an algorithm that provides for acquiring 2 or more lateral data points at a first position, and at least a second position within a ROI. In some embodiments, a subject computer program product comprises an algorithm that provides for correction or adjustment of the tip position, based on the cross-sectional profile data points. For example, where one or more cross-sectional profile data points indicate that the tip is off the “peak” of the parabolic cross-sectional profile of the immobilized ROI, the computer program product provides for adjustment of the tip position such that it is re-centered on the peak.

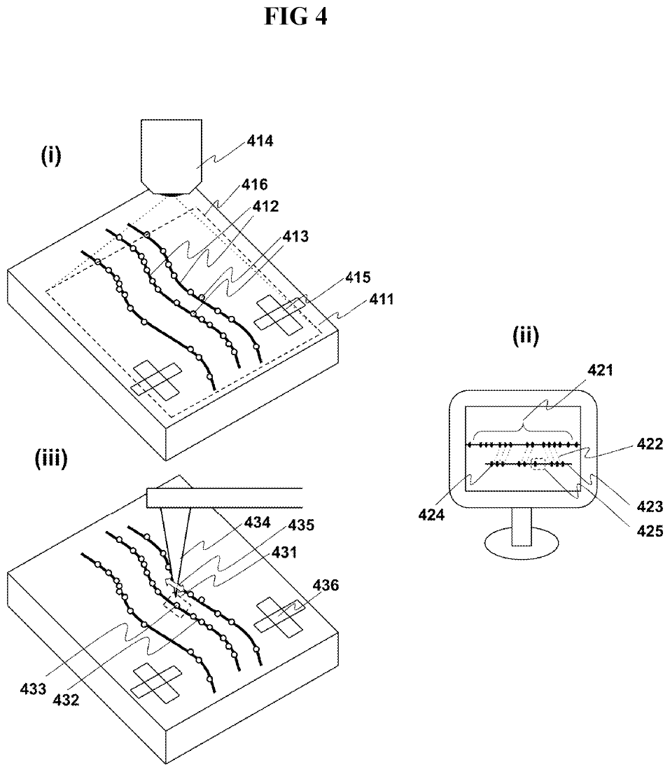

FIG. 4 demonstrates a workflow embodiment, where-by the fluorescent interrogation process is used to find ROI(s), and thus filter out the molecule(s) with no ROI(s), and so target the scanning of the contact probe to only the regions of the surface that contain ROI(s). In this embodiment workflow, at time point (i) a substrate or open fluidic device (411) is prepared with combed long nucleic acid molecules from a sample (412) that are modified with bound fluorescent labelling bodies (413) that produce a physical map along the length of each molecule. An optical interrogation system (414) captures a large field of view (FoV) fluorescent and brightfield image of the substrate surface (416). Image recognition is then employed to identify the florescent signature of each molecule's physical map to generate a digitized version, and to identify the bright field location of the fiducials (415) patterned on the surface of the substrate, providing reference positions for defining an xy plane coordinate system. Next (ii), each molecule's digitized physical map (423) is aligned (422) to one or more references (421) to identify any ROI(s) within the sample. In this example, an analysis of an alignment of a molecule to a reference identifies an insertion in the molecule with sufficient confidence that an ROI is flagged for the insert (425). With the ROI identified, (iii) the substrate or open fluidic device is positioned such that the ROI (431) is directly underneath the contact probe (434), and the contact probe system is aware of the coordinate system on the surface defined the by fiducials (436). The contact probe then performs a high-resolution raster scan (435) of the ROI.

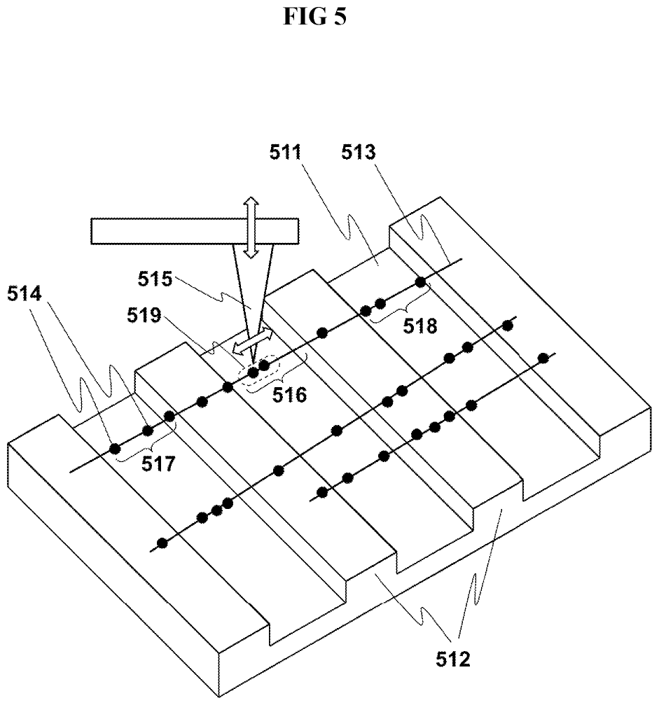

In some embodiments, at least a portion of an ROI within a long nucleic acid molecule to be interrogated by the contact probe device is physically suspended between two physical points that are topologically prominent on the surface of a substrate or open fluidic device. In the preferred embodiment, the suspended portion of the molecule is under tension. Referring to FIG. 5, long nucleic acid molecules (513) with bound labelling bodies (514) are combed, or transfer combed, to the substrate or open fluidic device (511) such that the elongated molecules are brought into contact with a collection of contact points that are topologically prominent features, in this described embodiment shown as bars (512), onto which the molecules are immobilized, yielding regions of the molecules (516, 517, 518) that are suspended above the substrate. After optical interrogation of the molecule to generate its respective digitized physical map from the labelling bodies, an ROI within the molecule is identified via alignment of the physical map to a reference. The contact probe is then brought into contact with the molecule to interrogate the ROI. In some embodiments, the long molecule does not comprise a physical map, but only a fluorescent signature to allow for its identification within x,y,z space to allow for placement of the contact probe to a suspended portion of the molecule. In some embodiments, the contact probe may interrogate a portion of the suspended long nucleic acid molecule that does not comprise an ROI.

In the preferred embodiment, the open fluidic device or substrate physically engages with a control instrument that interrogates the long nucleic acid molecule's optical physical map, is the same interrogation system instrument that directs the targeting of the contact probe to the ROI(s), such that all electrical mechanical systems within the instrument can share the same computer based system with coordinate space to target molecules and ROI(s) within the coordinate map. In some embodiments, the targeting of the contact probe interrogation is performed on a contact probe interrogation system instrument that is physically separate from the fluorescent interrogation system instrument, and fiducials on/within the open fluidic device or substrate are used to register the coordinate map between the instruments. In some embodiments, at least a portion of the sample itself may serve as fiducials.

In some embodiments, an ROI may be scanned multiple times. In some embodiments, a different scan parameters are used between scans. In some embodiments, the scan parameters may change between scans of the same ROI. Parameters that may change include: the particular sub-section(s) of the ROI, the addition of peripheral regions around the ROI, scan speed, scan velocity, scan direction, scan mode, scan force, scan resolution, scan frequency, contact probe tip type, the signal being measured, the contact probe operating mode, the contact probe tip functionalization. In some embodiments, the environment conditions may be altered between scans of the same ROI. In some embodiments, at least two different types of signals may be measured by the contact probe during the scan. Examples include height and conductance, height and lateral force, height and error.

For all embodiments an environmental condition may vary or change before, during, or after an interrogation of the ROI by the contact probe. For all embodiments, the physical location of at least a portion of the long nucleic acid molecule with respect to the substrate or open fluidic device may vary or change before, during, or after an interrogation of the ROI by the contact probe. In some embodiments, at least a portion of the long nucleic acid molecule is optically interrogated after said physical location change to register the new position(s).

In some embodiments, the targeted interrogation of the ROI by the contact probe allows for the physical or chemical manipulation of the ROI. In some embodiments, the contact probe can be used to physically separate from each other, move, or bring together into proximity, two or more sections of the long nucleic acid molecule. For example, to separate neighboring TAD boundaries within a chromosome, or to bring two non-proximal TAD boundaries into proximity together. In some embodiments, a binding event of a body to the long nucleic acid molecule may be facilitated prior, during, or after the physical manipulation of the ROI by the contact probe. In some embodiments, at least one reagent may be introduced to facilitate the binding event. In some embodiments of physical manipulation of the long nucleic acid molecule by the contact probe, the contact probe is functionalized. In some embodiments, the functionalization includes the binding of at least one reagent to the probe. In some embodiments, the contact probe may physically alter a higher order nucleic acid structure. In some embodiments, the contact probe may physically move at least a portion of a long nucleic acid molecule.

In some embodiments, unique barcodes are associated with the ROI(s) or subsets of ROI(s). The barcode can be the same for all ROI(s), but unique for the originating parent molecule, chromosome, cell, tissue, or patient. In some embodiments the barcode is known, in other embodiments it's randomly, or blindly assigned. The barcode may be associated to the ROI by binding to the ROI, either directly, or indirectly through an intermediary body. In the preferred embodiment, the barcode is attached directly or indirectly to a universal primer which then binds to the ROI. In some embodiments the unique barcode is associated with the ROI via physical confinement, for example within a shared droplet, or a shared entropic trap, or well. In some embodiments, the unique barcode is created from a unique combination of barcodes.

In some embodiments where-by a particular reagent is desired to interrogate a single-strand portion of the double strand long nucleic acid molecule, the reagent solution includes a recombinase enzyme to form D-loop as described by [Chen, 2016] such that a localized, stable de-natured portion can be maintained.

In some embodiments, the contact probe is functionalized such that the functionalized end of the contact probe can participate in a binding or enzymatic event with the nucleic acid within the ROI, either directly, or indirectly.

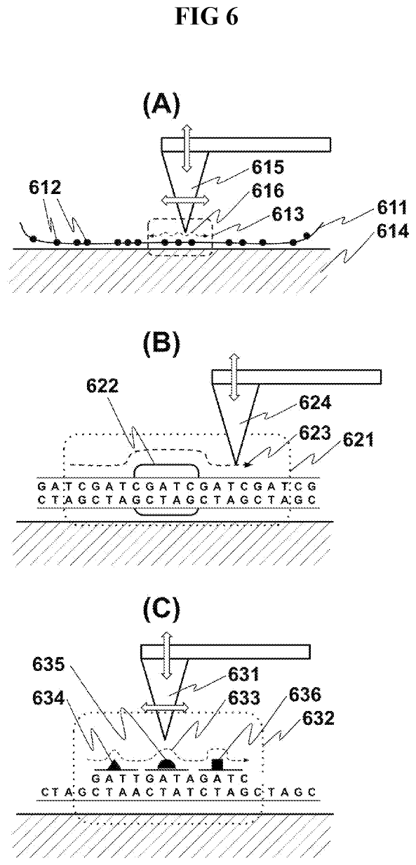

FIG. 6(A) demonstrates an embodiment where-by a contact probe (615) is brought into direct sensing contact with an ROI (613) portion of the long nucleic acid molecule bound with labelling bodies (612) and immobilized on the surface of a substrate or open fluidic chip (614). Depending on the scanning parameter selected, the contact probe can be used to discern individual nucleotides, individual k-mers (single base and double-stranded, where k can be 2, 3, 4, 5, 6, 7, 8, 9, or 10 bases in length), individual methylation status of a nucleotide, individual base-pairs, individual bound labelling bodies, and individual bound hybridized probes. In this embodiment, the contact probe is used to interrogate a more precise count and physical location of the bound labelling bodies by profiling the variation in the molecule's topology profile as the probe scans back and forth along the length of the ROI (616).

The embodiment shown in FIG. 6(B) demonstrates the detection of a bound labeling body (622) or higher order nucleic acid structure on a long nucleic acid molecule (621), where the z trace (623) along the length of the molecule generated by a scanning contact probe (624) shows a signal from the body or higher order nucleic acid structure. In some embodiments, the at least a portion of the ROI being interrogated by the contact probe may be double-stranded.

The embodiment shown in FIG. 6(C) demonstrates the interrogation of hybridized coded labelling bodies along the length of a single stranded portion of the ROI (632) by a contact probe (G31). In this example, the 3 hybridized coded labelling bodies, each hybridizing 4 nucleotides in length (634, 635, 636), and each comprising a different barcode or identifier that enables the labeling bodies to be differentiated from each other by the signal (633) generated by the contact probe. In this particular example, the barcode or identifier is associated with a specific sequence of nucleotides 4 bases in length, allowing for the determination of the underlying sequence of the ROI by the signal detected by the contact probe.

In some embodiments, at least a portion of the ROI within a long nucleic acid molecule presents a single-strand portion of the molecule. In some embodiments, the presentation of the single strand portion is facilitated at least in part by melting. In some embodiments, the melting is chemical enabled. In some embodiments, the melting is thermally enabled. In some embodiments, the presentation of the single strand portion is facilitated at least in part by introducing at least one single strand nick. In some embodiments, the presentation of the single strand portion is facilitated at least in part by an enzymatic process that includes stand-displacement.

In various embodiments of the invention, coded labeling bodies may be attached to the ROI prior to interrogation of the ROI by the contact probe. In some embodiments, coded labeling bodies may be attached to the ROI after ROI identification by optical interrogation of the physical map.

In various embodiments of the invention, the coded labeling bodies may comprise oligonucleotide probes, such as oligonucleotides of defined sequence. The oligonucleotides may be attached to a distinguishable barcode or identifiable body. In some embodiments, the identifiable body is identified by its physical size, or physical shape, or conductive property, or magnetic properties, or orientation with regards to the hybridization.

In various embodiments of the invention, oligonucleotide type coded labeling bodies may comprise DNA, RNA, or any analog thereof, such as peptide nucleic acid (PNA), which can be used to identify a specific complementary sequence in a nucleic acid. In certain embodiments of the invention one or more coded labeling body libraries may be prepared for hybridization to one or more nucleic acid molecules. For example, a set of coded labelling bodies containing all 4096 or about 2000 non-complementary 6-mers, or all 16,384 or about 8,000 non-complementary 7-mers may be used. If non-complementary subsets of oligonucleotide coded labeling bodies are to be used, a plurality of hybridizations and sequence analyses may be carried out and the results of the analyses merged into a single data set by computational methods. For example, if a library comprising only non-complementary 6-mers were used for hybridization and sequence analysis, a second hybridization and analysis using the same target nucleic acid molecule hybridized to those coded labeling bodies sequences excluded from the first library may be performed.

In some embodiments of the invention, the coded labelling body library may contain all possible sequences for a given oligonucleotide length (e.g., a six-mer library would consist of 4096 coded labeling bodies). In such cases, certain coded labelling bodies will form hybrids with complementary coded labelling body sequences. Such hybrids, as well as unhybridized coded labeling bodies, may be separated from coded labeling bodies hybridized to the target molecule using known methods, such as high performance liquid chromatography (HPLC), gel permeation chromatography, gel electrophoresis, ultrafiltration, rinsing, washing, or hydroxylapatite chromatography. Methods for the selection and generation of complete sets or specific subsets of oligonucleotides of all possible sequences for a given length are known. In various embodiments, coded labelling bodies of 2, 3, 4, 5, 6, 7, 8, 9, 10, 11, 12, 13, 14, 15, 16, 17, 18, 19, 20, 21, 22, 23, 24, 25 or more nucleotides in length may be used.

Each coded labeling body may incorporate at least one covalently or non-covalently attached barcode or identifier. The barcodes or identifier may be used to detect and/or identify individual coded labeling bodies. In certain embodiments of the invention each coded labeling body may have two or more attached barcodes or identifiers, the combination of which is sufficiently distinct to a particular coded labeling body that said coded labeling body can be differentiated from another coded labelling body. Combinations of barcodes and identifiers can be used to expand the number of distinguishable barcodes and identifiers available for specifically identifying a coded labeling body in a library. In other embodiments of the invention, the coded labelling bodies may each have a single barcode or identifier attached that is sufficiently distinct to a particular coded labeling body that said coded labeling body can be differentiated from another code labeling body. The only requirement is that the signal detected from each coded labeling body by the contact probe must be capable of distinguishably identifying that coded labeling body from a different coded labeling body.

In general, barcodes or identifiers will be covalently attached to the labeling body in such a manner as to minimize steric hindrance with the barcodes or identifier, in order to facilitate coded labeling body binding to a target long nucleic acid molecule. Linkers may be used that provide a degree of flexibility to the coded labeling body. Homo- or hetero-bifunctional linkers are available from various commercial sources.

In various embodiments of the invention, hybridization of a ROI to an oligonucleotide-based coded labeling body library may occur under stringent conditions that only allow hybridization between fully complementary nucleic acid sequences. It is understood that the temperature and/or ionic strength of an appropriate stringency are determined in part by the length of an oligonucleotide labeling body, the base content of the target sequences, and the presence of formamide, tetramethylammonium chloride or other solvents in the hybridization mixture. The ranges mentioned above are exemplary and the appropriate stringency for a particular hybridization reaction is often determined empirically by comparison to positive and/or negative controls. The person of ordinary skill in the art is able to routinely adjust hybridization conditions to allow for substantially exclusive hybridization between exactly complementary nucleic acid sequences to occur.

Once coded labeling bodies have been hybridized to a nucleic acid, adjacent coded labeling bodies may be ligated together using known methods.

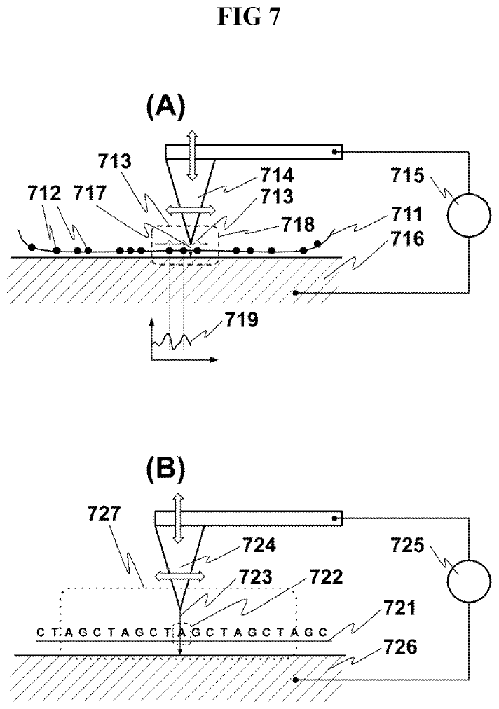

In some embodiments as demonstrated in FIGS. 7(A) and 7(B), a contact probe system capable of maintaining the height of the probe above the target ROI to be interrogated with a feedback system, while measuring an electrical signal is used to interrogate the ROI, thus generating both a z profile, and an electrical signal profile. In one embodiment, the contact probe is a scanning tunneling microscope. In another embodiment, the contact probe is a Conductive atomic force microscopy (C-AFM) system, and the electrical signal measured is the conductivity of the current path from the tip of the contact probe through the ROI sample, and into the underlying substrate or open fluidic device, for a fixed, or time-varying applied voltage.

FIG. 7(A) demonstrates an embodiment where a long nucleic acid molecule (711) combed on a substrate or open fluidic device (716), where said molecule is bound with labeling bodies (712) that form a physical map on said molecule, and said physical map is optically interrogated and aligned to a reference to identify an ROI (718), and said ROI is then interrogated by a C-AFM contract probe (714). In this embodiment, the contact probe will interrogate the z height profile (713) and electrical conductivity profile (719) of the molecule with associated bound bodies within the ROI. In this embodiment, the current from the contact probe tip, through the molecule with bound bodies, and into the substrate or open fluidic device is measured by an SMU (715). FIG. 7(B) demonstrates an embodiment where a long nucleic acid molecule (721) combed on the surface of a substrate or open fluidic device (726) has at least a portion of ROI (727) with an exposed single-strand section (722), allowing for the contact probe (724) to interrogate and resolve single bases, or k-mers, bound labeling bodies, or higher order nucleic acid structures along the single-strand portion of the ROI. In one embodiment, the contact probe (724) is used to interrogate the variation in measured current (723) along the length of the ROI (727). This measured current may be compared or aligned to a database of reference currents for known sequences.

In some embodiments, the contact probe may measure an electrical signal associated with a single nucleotide, or a base-pair, or a k-mer, or a bound labeling body, or a hybridized labeling body with a barcode or identifier that is associated with the specific hybridization sequence, or a higher order nucleic acid structure. In all previous embodiments, the electrical signal measured may vary with the presence, lack, or physical configuration of the objecting being measured with respect to the ROI's originating long nucleic acid molecule.

In some embodiments, the electrical properties of single nucleotides or base-pairs within the ROI can be altered by modifying the ROI to incorporate modified nucleotides with distinct electrical properties.

In the preferred embodiments, the SMU (715 or 725) provides the bias voltage and modulation waveform supplied to the contact probe tip. The SMU is also used to measure the time-dependent tunneling current variations. The acquired tunneling current signals can be stored in an optional data storage device for later analysis, or processed immediately using a high throughput, real-time method. In either approach, the acquired tunneling current signals are processed and can then be aligned or compared to a predetermined signal or signals characteristic of or associated with a known or simulated ROI to determine the degree of similarity.

According to quantum mechanics, the tunneling current, is a linear function of the bias voltage, so that the tunneling conductance, is a constant for a given orientation and composition of the portion of the ROI being interrogated by an electrical signal with respect to the contact probe tip. This approximation is accurate only for low bias voltages since it does not include effects attributable to the internal states of the portion of the ROI being interrogated. Taking into account the internal states, the tunneling conductance is modified when the energy of the tunneling electron is in the vicinity of Δεij≡εi−εj, where εi and εj are the energies of the state |i> and the state |j> of the portion of the ROI being interrogated, respectively. A corresponding “resonance voltage” can be determined from the variation of the tunneling conductance, and can be used to identify the portion of the ROI being interrogated.

Characteristics attributable to inelastic electron tunneling are observable and useful in deriving identification information. In this process, the second derivative of the tunneling current with respect to the bias voltage shows a detectable peak at the “resonance voltage.” (see “Single-Molecule Vibrational Spectroscopy and Microscopy”, B. Stipe, et al., Science, Vol. 280, pp. 1732-1735, 12 Jun. 1998, incorporate herein by reference.)

In some embodiments the physical map comprises a labeling body that is comprised of an affinity tag or hapten such as biotin and a complementary body is combined with the labeled sample to create a mass that can be detected by an AFM tip.

In other embodiments a nanoparticle such as a CdSe/Zn quantum dot or a gold nanoparticle is prepared with a complementary affinity moiety such as streptavidin and the nanoparticles are combined with the labeled nucleic acid and subsequently washed to preserve specific interactions. Larger nanoparticles are easier to detect with AFM but have reduced ability to physically make contact with the deposited nucleic acids.

In some embodiments the same labeling bodies used for fluorescence determination of a physical map are also used to create a fine-scale physical map by means of near-field scanning microscopy with fluorescence. In other embodiments, gold nanoparticles are attached to labelling bodies, such as binding with oligos containing thiolated linkers that are covalently bonded to the gold nanoparticles. Small nanoparticles are visualized using darkfield scattering microscopy to create a physical map and are then subsequently interrogated at high resolution using Scattering-type scanning near-field optical microscopy (s-SNOM), where individual particles are visible.

In some embodiments DNA is labeled by binding with DNA bending proteins such as IHF (E. coli), HU (B. stearothermophilus) und TF1 (B. subtilis). The fine scale mapping is performed by AFM imaging of DNA to detect sharp bends in the contour of the DNA.

In some embodiments, the physical position of the tip (in x-y) may follow the path coordinates of the ROI determined from the fluorescent physical map. In some embodiments, the physical position of the tip (in x-y) may dwell, or circle, or scan perpendicular to the local axis of the molecule.

In some embodiments, the at least a portion of the long nucleic acid molecule may be exposed to a solution, a reagent, a photon of a certain wavelength, or an environmental condition after the generating of the fluorescent physical map, but before the interrogation with the contact probe, or during the interrogation with the contact probe. In particular, in some embodiments, an additional labelling body may be bound to the molecule allowing for additional, or a higher resolution physical map to be generated within at least a portion of the ROI by the contact probe interrogation. In some embodiments, a least a portion of the ROI may be processed to allow for greater ease of contact probe access to a single-strand portion of the ROI. Such processes may comprise nicking of the double-strand molecule in at least one location, thermally melting at least a portion of the double strand molecule, chemically or enzymatically melting at least a portion of the double strand molecule.

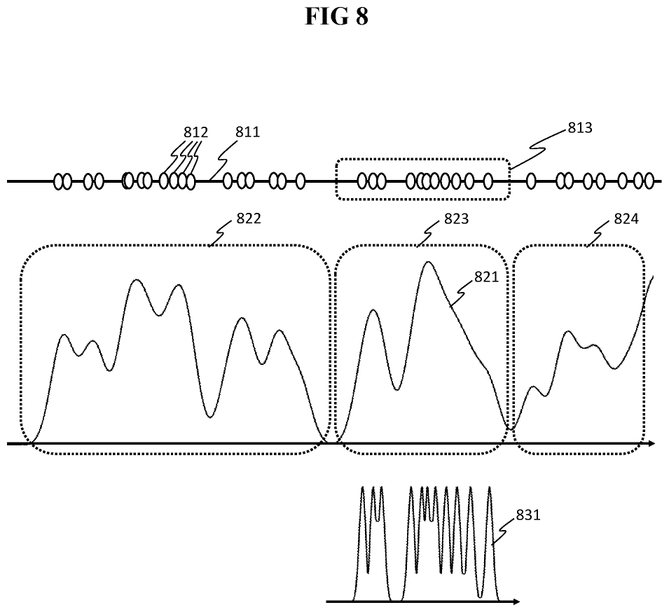

FIG. 8 demonstrates an embodiment where-by the contact probe is used to generate a topological physical map that is of a much higher resolution than the fluorescent physical map within the ROI. In such an embodiment, the fluorescent labelling bodies (812) are bound along the length of the long nucleic acid molecule (811), such that after optical interrogation of the molecule, a digitized representation of the physical map from the fluorescent signal along the length of molecule is generated (821), after said map under-going processing for stretch correction of the molecule and noise reduction. In this embodiment, the physical map is a melt map, and the labelling bodies are intercalating dyes bound to regions of the molecule with relatively high GC content. In this example embodiment, the physical map can be aligned to a melt map in-silco reference where regions 822 and 824 of the physical map align with high confidence to a reference, however section 823 cannot be aligned with confidence due to its lack of sufficient unique content to align to any location in a reference with confidence. To further elucidate the genomic content of region 823, this region is tagged as ROI for further investigation with a contact probe, where the interrogation of this ROI by contact probe is able to identify the individual label bodies along the length of the molecule, effectively converting the ROI's analog fluorescent signature physical melt map into a digital signature physical melt map, thus providing a much richer and higher resolution physical map of this ROI. This high-resolution ROI physical map can then be aligned to a suitable collection of references to better assess the genomic nature of the ROI.

In some embodiments, the contact probe is used to interrogate higher order structure within the ROI. In particular, in some embodiments, the contact probe is used to elucidate the nature of various topological structures which may not be resolvable via fluorescent interrogation. Such structures may comprise loops, knots, folds, forks, bubbles. In some embodiments, the interrogation of the higher order structure may comprise a 3D map of the ROI, including any bound labeling bodies and/or binding proteins or enzymes.

In some embodiments, the contact probe is used to interrogate the topological nature of a long nucleic acid molecule, for example, to determine if the molecule has loops, or is circular in nature. In one particular embodiment, the contact probe is used to identify circular ecDNA molecules. In the preferred embodiment, the contact probe is used to identify the ecDNA from other non-circular long nucleic acid molecules. In some embodiments, the ecDNA and non-circular long nucleic acid molecules all originate from the same cell.

A region of interest is identified for further in-depth analysis through a number of approaches as disclosed herein. A region of interest may be identified by comparison to a reference physical map or by direct identification within a sample physical map as discussed herein. Alternately or in combination, in some cases a region of interest is identified by the detection of an associated Landmark in a physical map. The landmark is variously coequal to, overlapping with, or distal to a region of interest. A landmark may indicate the presence of a local ROI such as a disease locus, variable region, SNP, or other ROI of relevance to a disease, phenotype or other condition. A landmark is often selected as a readily identifiable feature in a physical map that may point to a less readily identifiable ROI, such as an ROI that is distinguishable only upon investigation at a higher level of resolution or using an alternate physical mapping approach relative to an approach or method used to establish an initial physical map. Exemplary landmarks are loop structures, large GC or AT regions, distinct heterochromatin or euchromatin regions, or other readily distinguishable physical map features that may help one identify or locate a region of interest for subsequent analysis as disclosed herein.

In some cases, a Landmark is a feature in a physical map that is localized nearby a region that contains information accessible to high resolution interrogation. The Landmark can be identified solely from experimental data, from a match to a reference, a combination of partial match to a reference and partial discordance from the same or different reference and from a match to a reference in combination with prior knowledge of the presence or absence of a disease or phenotype, and also from a combination of a known position on a reference with knowledge of structural variability.

Examples of purely experimentally determined Landmarks are not limited to the identification of regions of chromatin that have a level of activity or repression that is above, at or below reference levels of activity, regions of DNA that exhibit a highly looped or condensed structure measured relative to a previously determined expectation of higher order structure, density of loops, gross topology of chromatin that is linear, circular, linear with loops, circular with supercoiling or other predetermined topological structures. Extended chromatin that exhibits bends greater, less than or equal to one or more threshold angles can be a landmark. Further examples of experimentally determined Landmarks include first acquiring a multiplicity of physical maps, before subjecting the maps to a pairwise association analysis to identify cluster of the maps such that each cluster represents a substantially similar portion of chromatin, for example regions of duplicated DNA present on different chromosomes or repeated sequences within the same chromosomal region. Other examples include DNA or chromatin molecules that are in a size range that is consistent with expectations, such as bodies of chromatin smaller than 5 Mbp.

Examples of referenced matched Landmarks are not limited to the locations of telomeric regions of chromosomes known to exist in proximity to a location on a physical map and extended in a direction closer or further away from the first landmark as determined by the presence of a second landmark such as a centromere or region known to be in proximity to the first landmark. Other examples include entire specific chromosomes, specific p arms or q arms of entire chromosomes, or extrachromosomal bodies such as ecDNA, or molecules that do not contain a centromere within the extent of observed molecule, or contain more than one centromere per molecule. Other examples include multiple degenerate Landmarks or features of physical maps across multiple portions of the reference that are substantially the same or similar or difficult to distinguish and can be distinguished by higher resolution probing of the region of interest.

Examples of reference+prior knowledge Landmarks include the location of a gene product or transcription initiation site that is located next to a region that contains a variable number of repeats that is in between an enhancer region that influences the expression of the gene. Other examples include sites bearing evidence of genomic insertions such as viral DNA insertions or genetic engineering such as CRISPR mediated gene editing, and prior knowledge of the anticipated structure or sequence of the inserted DNA. Other examples include regions of stable or relatively stable DNA that are known to be adjacent to regions of high structural variability.

Numbered Embodiments. The disclosure herein is further delineated by the following numbered embodiments. 1. A method of characterizing a region of interest of a nucleic acid molecule, comprising i) attaching the nucleic acid molecule to a surface of at least one point on the nucleic acid ii) determining a physical map of at least a portion of the nucleic acid molecule iii) comparing the physical map of at least a portion of the nucleic acid molecule to a Reference to identify a segment of the physical map that has a co-relationship to the at least a segment of the Reference iv) correlating the segment of the physical map of at least a portion of the nucleic acid molecule that differs from the correlating Reference to a region of interest on the nucleic acid molecule; v) subjecting the region of interest on the nucleic acid molecule to a second physical characterization. 2. The method of any of the previous embodiments, wherein the surface is exposed. 3. The method of any of the previous embodiments, wherein the surface is not interior to a flow cell. 4. The method of any of the previous embodiments, wherein the surface is not interior to a fluidic device. 5. The method of any of the previous embodiments, wherein the surface is accessible to exterior mechanical manipulation. 6. The method of any of the previous embodiments, wherein attaching the nucleic acid molecule comprises binding a chromatin constituent associated with the nucleic acid molecule to a chromatin constituent affinity partner. 7. The method of any of the previous embodiments, wherein attaching comprises immobilizing the nucleic acid to the surface. 8. The method of any of the previous embodiments, wherein determining a physical map of at least a portion of the nucleic acid molecule comprises determining an AT concentration of the at least a portion of the nucleic acid molecule. 9. The method of any of the previous embodiments, wherein determining a physical map of at least a portion of the nucleic acid molecule comprises determining a GC concentration of the at least a portion of the nucleic acid molecule. 10. The method of any of the previous embodiments, wherein determining a physical map of at least a portion of the nucleic acid molecule comprises determining a nucleic acid subsequence pattern for a recurring subsequence of the at least a portion of the nucleic acid molecule. 11. The method of any of the previous embodiments 10, wherein the nucleic acid subsequence pattern comprises a repeat element pattern. 12. The method of any of the previous embodiments 11, wherein the repeat element comprises a transposon. 13. The method of any of the previous embodiments 11, wherein the repeat element comprises a retroelement. 14. The method of any of the previous embodiments 11, wherein the repeat element comprises an Alu repeat. 15. The method of any of the previous embodiments 11, wherein the repeat element comprises an octomer. 16. The method of any of the previous embodiments 11, wherein the repeat element comprises a hexamer. 17. The method of any of the previous embodiments, wherein determining a physical map of at least a portion of the nucleic acid molecule comprises determining a nucleic acid higher order structure pattern. 18. The method of any of the previous embodiments, wherein the nucleic acid higher order structure pattern comprises a nucleic acid knot pattern. 19. The method of any of the previous embodiments, wherein the nucleic acid higher order structure pattern comprises a nucleic acid binding protein binding pattern. 20. The method of any of the previous embodiments, wherein the nucleic acid higher order structure pattern comprises a topological pattern. 21. The method of any of the previous embodiments, wherein determining a physical map of at least a portion of the nucleic acid molecule comprises determining a nucleic acid associate protein binding pattern. 22. The method of any of the previous embodiments, wherein the nucleic acid associate protein binding pattern is a chromatin protein binding pattern. 23. The method of any of the previous embodiments, wherein the nucleic acid associate protein binding pattern is an exogenous protein binding pattern. 24. The method of any of the previous embodiments, wherein the nucleic acid associate protein binding pattern is a CRISPR protein complex binding pattern. 25. The method of any of the previous embodiments, wherein the nucleic acid associate protein binding pattern is a transcription factor binding pattern. 26. The method of any of the previous embodiments, wherein the nucleic acid associate protein binding pattern is a histone binding pattern. 27. he method of any of the previous embodiments, wherein the nucleic acid associate protein binding pattern is a modified histone binding pattern. 28. The method of any of the previous embodiments, wherein determining a physical map of at least a portion of the nucleic acid molecule comprises determining a nucleic acid modification pattern. 29. The method of any of the previous embodiments 28, wherein the nucleic acid modification pattern results from contacting bound labelling bodies. 30. The method of any of the previous embodiments 28, wherein the nucleic acid modification pattern is a DNA methylation pattern. 31. The method of any of the previous embodiments, wherein determining a physical map of at least a portion of the nucleic acid molecule does not comprise sequencing the at least a portion of the nucleic acid molecule. 32. The method of any of the previous embodiments, wherein determining a physical map of at least a portion of the nucleic acid molecule requires no more than 1 second. 33. The method of any of the previous embodiments, wherein determining a physical map of at least a portion of the nucleic acid molecule requires no more than 1/100 of a second. 34. The method of any of the previous embodiments, wherein the comparing comprises aligning. 35. The method of any of the previous embodiments, wherein aligning the physical map of at least a portion of the nucleic acid molecule to a reference comprises identifying a segment of the physical map of at least a portion of the nucleic acid molecule that is absent from the reference. 36. The method of any of the previous embodiments, wherein aligning the physical map of at least a portion of the nucleic acid molecule to a Reference comprises identifying a segment of the physical map of at least a portion of the nucleic acid molecule that is inverted relative to the Reference. 37. The method of any of the previous embodiments, wherein aligning the physical map of at least a portion of the nucleic acid molecule to a Reference comprises identifying a segment of the physical map of at least a portion of the nucleic acid molecule is translocated relative to the Reference. 38. The method of any of the previous embodiments, wherein aligning the physical map of at least a portion of the nucleic acid molecule to a Reference comprises identifying a segment of the physical map of at least a portion of the nucleic acid molecule that that is duplicated relative to the Reference. 39. The method of any of the previous embodiments, wherein aligning the physical map of at least a portion of the nucleic acid molecule to a Reference comprises identifying a segment of the physical map of at least a portion of the nucleic acid molecule that differs by at least 5% relative to the Reference 40. The method of any of the previous embodiments, wherein aligning the physical map of at least a portion of the nucleic acid molecule to a Reference comprises identifying a segment of the physical map of at least a portion of the nucleic acid molecule that that differs by at least 10% relative to the Reference 41. The method of any of the previous embodiments, wherein aligning the physical map of at least a portion of the nucleic acid molecule to a Reference comprises identifying a segment of the physical map of at least a portion of the nucleic acid molecule that differs by at least 20% relative to the Reference 42. The method of any of the previous embodiments, wherein aligning the physical map of at least a portion of the nucleic acid molecule to a Reference to identify a segment of the physical map of at least a portion of the nucleic acid molecule that differs by at least 50% relative to the Reference 43. The method of any of the previous embodiments, wherein the Reference comprises a predictive physical map. 44. The method of any of the previous embodiments, wherein the Reference is derived from a nucleic acid sequence. 45. The method of any of the previous embodiments, wherein the nucleic acid sequence is a genomic sequence. 46. The method of any of the previous embodiments, wherein the nucleic acid sequence is derived from a reference organism. 47. The method of any of the previous embodiments, wherein the nucleic acid sequence is derived from a cancer-free cell. 48. The method of any of the previous embodiments, wherein the Reference is previously obtained. 49. The method of any of the previous embodiments, wherein the Reference is concurrently obtained. 50. The method of any of the previous embodiments, wherein the Reference is obtained from a tissue distal to a tissue from which the nucleic acid molecule is obtained. 51. The method of any of the previous embodiments, wherein the tissue and the nucleic acid are obtained from a common individual. 52. The method of any of the previous embodiments, wherein the tissue is disease free. 53. The method of any of the previous embodiments, wherein the tissue is cancer free. 54. The method of any of the previous embodiments, wherein the nucleic acid molecule is obtained from a cancerous cell. 55. The method of any of the previous embodiments, wherein the tissue is cancerous. 56. The method of any of the previous embodiments, wherein the tissue exhibits a disease. 57. The method of any of the previous embodiments, wherein the nucleic acid molecule is obtained from a healthy cell. 58. The method of any of the previous embodiments, wherein the nucleic acid molecule is obtained from a disease-free cell. 59. The method of any of the previous embodiments, wherein the tissue and the nucleic acid differ in age. 60. The method of any of the previous embodiments, wherein the tissue is a preserved tissue. 61. The method of any of the previous embodiments, wherein the nucleic acid is from a later obtained cell. 62. The method of any of the previous embodiments, wherein the nucleic acid is from an earlier obtained cell. 63. The method of any of the previous embodiments, wherein correlating the segment of the physical map of at least a portion of the nucleic acid molecule that differs from the Reference to a region of interest on the nucleic acid molecule comprises identifying a location of the region of interest on the nucleic acid molecule on the surface. 64. The method of any of the previous embodiments, wherein subjecting the region of interest on the nucleic acid molecule to a second physical characterization comprises removing a cover slip covering the nucleic acid molecule. 65. The method of any of the previous embodiments, wherein subjecting the region of interest on the nucleic acid molecule to a second physical characterization occurs on an exposed area of the surface. 66. The method of any of the previous embodiments, wherein subjecting the region of interest on the nucleic acid molecule to a second physical characterization comprises generating a second physical characterization of the region of interest on the nucleic acid molecule. 67. The method of any of the previous embodiments, wherein the second physical characterization depicts a characteristic different from that initially characterized. 68. The method of any of the previous embodiments, wherein the second physical characterization depicts an AT pattern. 69. The method of any of the previous embodiments, wherein the second physical characterization depicts a purine/pyrimidine pattern. 70. The method of any of the previous embodiments, wherein the second physical characterization depicts a protein binding pattern. 71. The method of any of the previous embodiments, wherein the second physical characterization depicts secondary structure concentration. 72. The method of any of the previous embodiments, wherein the second physical characterization depicts a histone modification pattern. 73. The method of any of the previous embodiments, wherein the second physical characterization depicts a nucleic acid modification pattern. 74. The method of any of the previous embodiments, wherein the second physical characterization depicts an octomer distribution pattern. 75. The method of any of the previous embodiments, wherein the second physical characterization depicts a hexamer distribution pattern. 76. The method of any of the previous embodiments, wherein the second physical characterization depicts a transposable element pattern. 77. The method of any of the previous embodiments, wherein the second physical characterization comprises a nucleic acid probe binding pattern. 78. The method of any of the previous embodiments, wherein the second physical characterization presents the number of repeats of a repeated element. 79. The method of any of the previous embodiments, wherein the nucleic acid probe binding pattern is assayed using a fluorophore bound to a nucleic acid probe. 80. The method of any of the previous embodiments, wherein the nucleic acid probe binding pattern is assayed using a barcode tag bound to a nucleic acid probe. 81. The method of any of the previous embodiments, wherein the second physical characterization comprises obtaining a nucleic acid sequence. 82. The method of any of the previous embodiments, wherein the second physical characterization comprises subjecting the region to a contact probe. 83. The method of any of the previous embodiments, wherein the contact probe determines a nucleic acid sequence for at least a portion of the region. 84. The method of any of the previous embodiments, wherein the contact probe is an atomic force microscopy probe. 85. The method of any of the previous embodiments, wherein the contact probe determines a position of the region in an axis perpendicular to the region. 86. The method of any of the previous embodiments, wherein the second physical characterization comprises physically manipulating the region. 87. A method of analyzing a nucleic acid, comprising generating a physical map of the nucleic acid in no more than 1 second, comparing the physical map to a reference, and generating a second physical map of a portion of the nucleic acid. 88. The method of any of the previous embodiments, wherein the portion of the nucleic acid that differs from the reference is inverted relative to the reference. 89. The method of any of the previous embodiments, wherein the portion of the nucleic acid that differs from the reference is translocated relative to the reference. 90. The method of any of the previous embodiments, wherein the portion of the nucleic acid that differs from the reference is duplicated relative to the reference. 91. The method of any of the previous embodiments, wherein the portion of the nucleic acid that differs from the reference is absent from the reference. 92. The method of any of the previous embodiments, wherein the second physical map comprises a sequence of the portion of the nucleic acid that differs from the reference. 93. The method of any of the previous embodiments, wherein the sequence is determined in situ. 94. The method of any of the previous embodiments, wherein the sequence is determined by direct manipulation of the nucleic acid on the surface. 95. The method of any of the previous embodiments, wherein the sequence is determined using atomic force microscopy. 96. The method of any of the previous embodiments, wherein the sequence is determined using hybridization to a probe of known sequence. 97. The method of any of the previous embodiments, wherein the nucleic acid is fixed to a surface. 98. The method of any of the previous embodiments, wherein the surface is exposed. 99. The method of any of the previous embodiments, wherein the surface is not a flow cell interior. 100. The method of any of the previous embodiments, wherein the surface is accessible to physical manipulation. 101. The method of any of the previous embodiments, wherein the surface is covered by a removable cover slip. 102. A system for analyzing a nucleic acid comprising an open surface to which the nucleic acid is attached (immobilized), a lens for capturing an optical signal indicative of a physical map of the nucleic acid, and a contact probe for determining a characteristic of a subregion of the nucleic acid. The system of any of the previous embodiments, wherein the system incorporates an element of a method recited in this section or elsewhere throughout the present disclosure. 103. The system of any of the previous embodiments, comprising a stored reference physical map and a processing unit to compare the stored reference physical map to a nucleic acid physical map generated from the fluorescence. 104. The system of any of the previous embodiments, wherein the processing unit is configured to identify a difference between the stored reference physical map to the nucleic acid physical map generated from the optical signal. 105. A method of analyzing a nucleic acid, comprising a. attaching the nucleic acid to a surface; b. determining a physical map for at least a portion of the nucleic acid; c. using the physical map to identify a region of interest in the nucleic acid molecule; and d. subjecting the region of interest on the nucleic acid molecule to a second physical characterization. 106. The method of any of the previous embodiments, wherein using the physical map to identify a region of interest comprises comparing the physical map to a reference, and correlating a landmark on the reference to the physical map to identify a region of interest in the nucleic acid molecule. 107. The method of any of the previous embodiments, wherein the physical map does not differ from the reference. 108. The method of any of the previous embodiments, wherein the physical map differs from the reference. 109. The method of any of the previous embodiments, wherein the landmark is a known variable region on the reference. 110. The method of any of the previous embodiments, wherein the landmark aligns with the region of interest. 111. The method of any of the previous embodiments, wherein the landmark is removed a known distance from a region on the reference that corresponds to the region of interest on the nucleic acid molecule. 112. The method of any of the previous embodiments, wherein the second physical characterization comprises a higher resolution map at the region of interest on the nucleic acid molecule than the physical map. 113. The method of any of the previous embodiments, wherein the second physical characterization comprises a nucleic acid sequence of the region of interest of the nucleic acid. 114. The method of any of the previous embodiments, wherein the second physical characterization comprises determining a second physical map of the region of interest. 115. The method of any of the previous embodiments, wherein determining the physical map on the nucleic acid molecule does not preclude subjecting the region of interest on the nucleic acid molecule to a second physical characterization. 116. The method of any of the previous embodiments, wherein the reference is a physical map of a nucleic acid from a non-diseased cell. 117. The method of any of the previous embodiments, wherein the reference is a physical map of a nucleic acid from a diseased cell. 118. The method of any of the previous embodiments, wherein the reference is a physical map of a nucleic acid from a cell exhibiting a phenotype of interest. 119. The method of any of the previous embodiments, wherein the reference is derived from a nucleic acid sequence. 120. The method of any of the previous embodiments, wherein the nucleic acid sequence is a genomic nucleic acid sequence. 121. A method of analyzing a population of nucleic acids, comprising generating distinct physical maps of members of the population of nucleic acids, and directing a contact probe to a region within at least one physical map, wherein at least one physical map is generated per molecule within the population per second. 122. The method of any of the previous embodiments, wherein the physical maps are generated successively, or alternately, concurrently. 123. The method of any of the previous embodiments, comprising generating second physical maps of a portion of at least some of the nucleic acids. 124. The method of any of the previous embodiments, wherein the second physical maps represent subsets of the distinct physical maps. 125. The method of any of the previous embodiments, wherein the second physical maps target regions identified through comparison to at least one reference. 126. The method of any of the previous embodiments, wherein the second physical maps target regions that differ among the distinct physical maps of members of the population of nucleic acids. 127. A method of characterizing a region of interest of a nucleic acid molecule, comprising a. attaching the nucleic acid molecule to a surface of at least one point on the nucleic acid b. determining a physical map of at least a portion of the nucleic acid molecule c. identifying at least one landmark by comparing the physical map of at least a portion of the nucleic acid molecule to a reference d. calculating the spatial extent of a region of interest relative to the landmark e. subjecting the region of interest on the nucleic acid molecule to a second physical characterization. 128. The method of any of the previous embodiments, wherein attaching comprises immobilizing. 129. The method of any of the previous embodiments, wherein comparing comprises aligning. 130. The method of any of the previous embodiments, wherein calculating the spatial extent of a region of interest comprises calculating the smallest rectangle inclusive of two or more landmarks. 131. The method of any of the previous embodiments, wherein calculating the spatial extent of a region of interest comprises calculating the coordinates of an enclosed area containing the landmark whereby the landmark is not closer than 1 um to any point in the periphery. 132. The method of any of the previous embodiments, wherein calculating the spatial extent of a region of interest comprises calculating the coordinates of an enclosed area that is a fixed distance upstream or downstream of the landmark. 133. The method of any of the previous embodiments, wherein calculating the spatial extent of a region of interest comprises calculating the coordinates of an enclosed area based on a landmark and scaled by the observed distances between two or more landmarks. 134. The method of any of the previous embodiments, wherein calculating the spatial extent of a region of interest comprises calculating the coordinates of an enclosed area to be a fixed distance from a landmark and excluding regions devoid of nucleic acids. 135. The method of any of the previous embodiments, wherein identifying comprises finding regions of the physical map that differ from the Reference. 136. The method of any of the previous embodiments, wherein identifying comprises finding regions of the physical map that are similar to a specific portion of the Reference.

### EXAMPLES

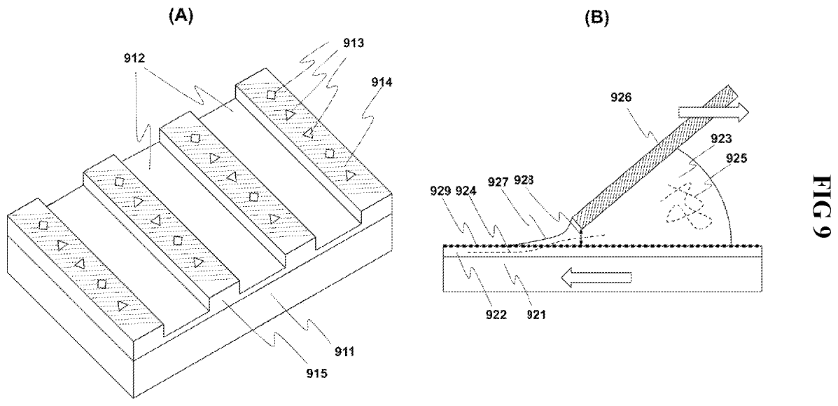

Example #1: Fabrication of an open fluidic device for combing DNA. As an initial proof of concept, a model system for an open fluidic device for preparation of long nucleic acid molecules for fluorescent and AFM interrogation is developed in a geometry similar to the embodiment shown in FIG. 9(A). The intended device geometries, including the channels into which the long nucleic acid molecules will be combed, and the fiducials for registering x-y coordinates, are first defined using a CAD software program such that a contact photomask can be specified for order from a mask vendor. Once the mask is obtained, a silicon wafer (911) with surface roughness less than 0.2 nm RMS is coated with a low pressure chemical vapor deposition (LPCVD) film of silicon oxide at a thickness of 50 nm, deposited at a temperature of 600 C. Next, a layer of positive photoresist is spin coated over the surface of the silicon oxide, and then prepared for exposure according to the resist manufactures instructions. Operating a mask aligner in contact mode, the resist on the wafer is exposed through the mask to UV light, after which the resist is developed according to the instructions and chemicals recommended by the manufacturer to remove the UV exposed resist and expose the silicon oxide film surface where the channels and fiducials will be formed. In this example, the channels are 1 micron wide, with pitch of 3 microns. Between the channels are various geometric shapes: circles, squares, triangles, etc (913) that can provide fiducials for registering ROIs between the optical and contact probe interrogation. The exposed silicon oxide is then anisotropically etched in reactive ion etcher (RIE) plasma consisting mostly of CHF3 as the etchant gas, approximately 45 nm deep into the 50 nm film of silicon oxide, using the photoresist as an etch mask. A final wet etch of diluted buffered hydrofluoric acid then used to etch the remaining silicon oxide in the channel, with minimal impact on the underlying silicon wafer roughness. The photoresist is then removed in a solution according to the manufacturer's instructions. The silicon wafer is then thoroughly cleaned in a heated bath of Ammonium hydroxide and Hydrogen peroxide in water, and then a thin 5 nm film of silicon oxide is thermally grown on the exposed silicon by dry thermal oxidation at 1100 to maintain a surface roughness <0.3 nm RMS.

Next, the top silicon oxide surface (914) is treated with a hydrophobic silane monolayer to silanize the surface. This will both allow for the receding meniscus of solution to wet into the channels, and for containment of solution and long nucleic acid molecules within the channels. Silane treatment is performed by contact printing against a PDMS film that was previously submerged in a solvent of silane molecules, thus transferring the molecules to the elevated silicon oxide regions between the channels via direct physical contact. The contact printing does not modify the channels, which due to their depressed topography, retain the silicon oxide's hydrophilic nature. After a 50 C anneal for 1 hour, the device is ready for use, consisting of 1 micron wide hydrophilic channels (912) formed in silicon dioxide (915) separated from each other by a 2 micron wide topologically elevated spacer (914).

Example #2: Preparing combed DNA with optical maps in preparation for interrogation. Human genomic DNA is isolated from blood samples by embedding purified nuclei in low melting point agarose plugs [Zhang, 2012]. The sample is electroeluted into low salt denaturing buffer (0.1×TBE, 20 mM NaCl, 2% Beta-mercaptoethanol) with YOYO-1 at a ratio of 1 dye per 10 nucleotide pairs and incubated at 18 C overnight. The sample is diluted 1:1 with formamide with minimal manipulation and heated to 31 C for 10 minutes [Tegenfeldt, 2009, U.S. Pat. No. 10,434,512] before quenching on ice. The sample is immediately added to the device which is kept at temperature of 16-19 C.

Referring the example demonstrated in FIG. 9(B), approximately 5 micro litters of solution (923) that includes the long nucleic acid molecules (925) prepared in Example #2 is dispensed onto the surface of the open fluidic device (921) that was manufactured in Example #1. The device is positioned on a motorized translation stage equipped with glass blade (926) that is brought into contact with the solution, held a distance of 100 microns (928) from the surface of the open fluidic device, and maintained at a 45 degree angle with respect to the open fluidic chip surface. The glass blade is then moved across the surface of the open fluidic device at a speed of 25 microns per second via the motorized stage, combing the long nucleic acid molecules (924) into the channels (922) by the receding meniscus (927) of the sample solution moved by the blade, while avoiding contact with the topologically prominent spacers between the channels due to hydrophobic coating (929).

Example #3: Operating a control instrument for interrogating combed DNA. A control instrument consists of a precision motorized xyz stage capable of <0.1 micron positional movement accuracy over 100 mm of xy travel on to which the open fluidic device is positioned, and allows for the open fluidic device to be selectively positioned under an objective for optical interrogation or an AFM tip for contact probe interrogation. The optical interrogation system is capable of bright field and fluorescent imaging with a selection of different excitation wavelengths and dichroic filters. The objective consists of a CFI Apo TIRF 60XC oil immersion objective, and the camera consists of a QHYCCD QHY294M-PRO Camera with a Sony IMX492 sensor operated in 2×2 binning mode. The instrument has a field of view (FoV) of 190 um×250 um, allowing 750 kb of fully stretched DNA to be visualized with an optical resolution of 500 bp. The contact probe interrogation system consists of an AFM tip operating in non-contact or tapping mode with a silicon cantilever (resonance frequency=70 kHz, spring constant=0.4 N m−1, and tip radius=2 nm).

Once the open fluidic device with combed long nucleic acid molecules is loaded into the control instrument, software is used to autofocus the fiducials via image analysis of the fluidic device to register the positions of the fiducials in 3D space. Next, at least 2 fiducials are positioned under the AFM tip and rastered scanned by the AFM system via movement the of stage in the xy plane. This allows for fine-tuning of the registration of the relative fixed distance between the AFM and the objective, allowing for the xyz stage to translate any selected position on the fluidic device's surface between the objective's focal position and under the AFM tip with less than 1 micron positional error.

Next, the control instrument, switching between bright-field mode and fluorescent mode, images the combed long nucleic acid on the surface of the open fluidic chip by raster scanning, in steps equivalent to the optical FoV, and stitches the images together. The fluorescent images are used for molecule identification, and the overlapping bright-field mode images are used to capture the locations of the channels, and the fiducials within the channels.

The backbone of each combed molecule is then identified computationally from the stitched images, and a trace of the intensity profiles is generated in each channel. The traces are background subtracted and a cumulative brightness histogram is generated for each channel. The traces are normalized to generate a best estimate of GC content and the physical map of the DNA strand under analysis. A map of each position along the physical map of the long nucleic acid molecule to physical coordinates on the surface of the open fluidic device is also obtained.

The physical map of each molecule is aligned to a pre-computed reference physical maps that are derived from sequences of the human genome assembly GRCh37 analyzed for melting state by the method of [Tostesen, 2005]. Reference map segments are sampled at intervals corresponding to one pixel of detected image and each pixel worth of GC ratio information is normalized as a signed 8 bit integer, where −128 represents 100% AT, 127 represents 100% GC. The reference map is pre-computed for a variety (up to 20) DNA stretch ratios, so the same sequence is present multiple times. Observed maps are compared with the physical map references in two steps, first each molecule is artificially segmented into 32 pixel segments starting every other pixel. This corresponds to approximately 8-13 kbp depending on DNA stretch. The dot product of each segment and a 32 pixel tile of the reference map segments is computed. The top 4 k matches are passed to the second stage, which repeats the dot product on neighboring regions in both the map and the sample and scores them with a Smith-Waterman algorithm to permit local insertions and deletions. Detection cutoffs are determined empirically.

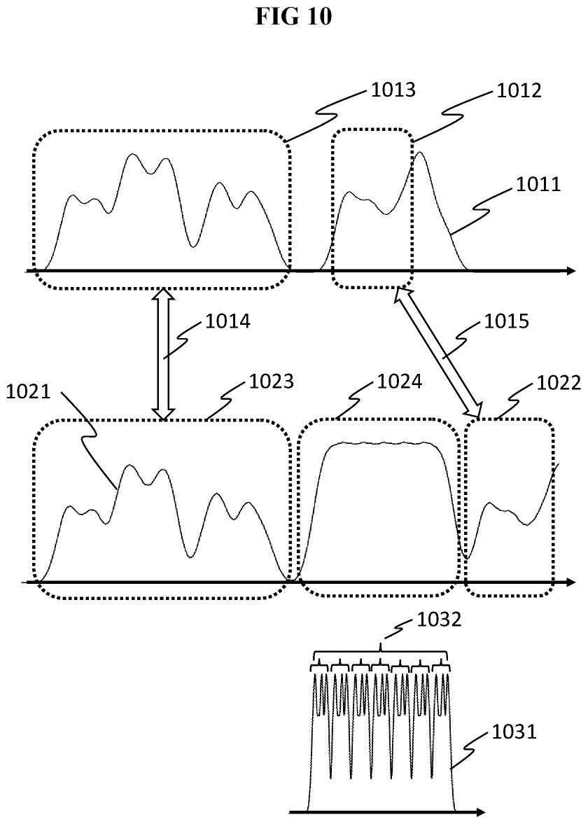

In this example demonstrated in FIG. 10, an analysis of the physical map of one molecule (1021) demonstrates an observed insertion of nucleic acid of approximate length 1200 base-pairs +/−500 bp (1024), when aligned to the in-silico reference (1011), demonstrated by the high-confidence alignment (1014) between regions 1013 of the reference and 1023 of the molecule's digitized optical physical map, and with the high-confidence alignment (1015) between regions 1012 of the reference and 1022 of the molecule's digitized physical map. In this example, an analysis of the portion of the digitized optical physical map within the insertion (1024) demonstrates a mostly uniform fluorescent signature, suggesting a uniform density of GC content within the insertion, within the 500 bp resolution of the optical interrogation system. This insertion is then flagged as an ROI for further investigation by the AFM as it lacks sufficient uniqueness to determine any additional genomic information of the ROI's nature via additional alignment analysis with a reference, in addition, the ROI's location within the genome as determine by the previous alignment, is in close proximity to regions of the genome known to be associated with specific diseases that may be relevant to the patient's health from which the sample originated. In this example, the ROI is 2.5 microns long, along the length of the molecule, where the insertion is 0.5 microns in length, and the ROI extends by 1 micron on both sides.

The xy stage then positions the ROI coordinates under the AFM tip, and first performs a fast, low-resolution tapping mode scan at 10 hz, 64 pixel/scan. Over a region of 5×5 microns, centered on the ROI to locate the channels and the fiducials between the channels, allowing the AFM to target the desired ROI location with less than 0.2 microns error in x and y direction. A high resolution scan 0.5 microns wide with 512 pixels at 0.5 hz is then made at the top of the ROI to register the location of the long nucleic acid strand, and once located, a high resolution raster scan of the ROI at 0.5 hz proceeds along the length of the ROI, performing multiple scans along each trajectory path to collect and process the data until the noise level falls below a required minimum. Using a combination of the coordinates of the ROI determined via the optical interrogation, and the on-the-fly contact probe data, the scanning parameters of the AFM tip are constantly adjusted with a feedback system to ensure as much as possible, that the scan direction is along the length of the molecule. As the molecule is scanned, the control instrument software follows the trace of the molecule along the backbone, and detects the signature of molecule topology changing in regions where YOYO dye is intercalated, producing a higher resolution physical map of the ROI where the location of individual dyes along the ROI can be registered. In this particular example, a high-resolution physical map (1031) generated by the AFM and processed to reduce the background noise of the surface and molecule itself, and enhance the detection of YOYO dyes bound to the ROI section of the molecule, shows evidence of a repeating region with 7 distinct copies.

Example 4: Oncogenic ecDNA pathology using combined atomic force microscopy and fluorescence imaging. A resection sample from a neuroblastoma patient is cryopreserved and transported to a pathology lab. Tissue is broken up into single nuclei by chopping, washing in Tween with salts and Tris (TST) buffer and filtered though a 35 um filter. Dilute nuclei are centrifuged down onto a silicon substrate patterned with regular fiducial markers and subjected to a cocktail of RNase H, lipase, hypotonic concentrations of monovalent salts and EDTA to loosen up the nucleus. The nuclei are quickly washed 10 mM MES pH 5.7 and the contents are combed onto the surface by removing the substrate from liquid at 20 um/s. The sample is dried in a gaseous nitrogen stream, washed thoroughly with 3:1 methanol:acetic acid and nitrogen dried again.

Immunofluorescence is used to detect regions of unusually active chromatin. Three categories of DNA corresponding to transcription start sites, active enhancers and gene bodies of actively transcribed genes are identified by fluorescent antibodies raised against H3K4me3 & H3K27ac, H3K4me1& H3K27ac and H3K36me3 respectively. Each category is labeled with a different sized quantum dot, which fluoresces at a wavelength corresponding to its size. The center wavelengths are 500 nm, 600 nm, and 750 nm. The substrate is additionally incubated non-specific intercalating DNA stain POPO-1, washed extensively, and subjected to a dehydration series of 70%, 85% and 100% ethanol.

The substrate is imaged with a combination of brightfield microscopy to register fiducial markers and multichannel fluorescence imaging to locate modified chromatin and intercalators on all DNA strands. The locations of the fluorescence are registered relative to the substrate fiducial markers. Landmarks are identified regions of overly bright fluorescence staining using empirical cutoffs. Extra weight is attributed to regions that exhibit overlapping fluorescence signals corresponding to activity in multiple categories. Regions of interest are enumerated for each landmark by calculating the smallest rectangle that contains the extent of the portion of the fluorescence that exceeds the empirical cutoff, and all non-specifically stained DNA that is contiguous with the landmark DNA strand within a 5 Mbp region.

An AFM tip is brought into contact with the extent of each region of interest, and the chromatin is scanned. For each piece of DNA it is determined whether the DNA is linear (likely chromosomal) or circular (likely ecDNA). The analysis is repeated for multiple nuclei, resulting in an analysis report quantifying the number of times the active chromatin was found in linear and circular forms, both in absolute counts and as a ratio to the total number of nuclei.

A fine scale topological map is constructed by scanning the contour of the chromatin, and counting the presence of loops. The quantum dots corresponding to transcription, gene body and enhancer sites are resolved by their respective sizes. The distances between transcription start sites and enhancers are measured both in physical distance across the substrate as well as contour distance along the DNA backbone, measured via the shortest path around any loops that are observed.

A report is generated, summarizing the statistics of circular DNA vs. linear, the number of active chromatin sites, the distances between start sites and enhances, and combinations thereof. Particular attention is given to circular DNA that is highly active and contains close transcription sites and enhances. The report is analyzed by a pathologist.

What is claimed is:

1. A method of characterizing a region of interest of a nucleic acid molecule, comprising
i) attaching the nucleic acid molecule to a surface of at least one point on the nucleic acid
ii) determining a physical map of at least a portion of the nucleic acid molecule
iii) comparing the physical map of at least a portion of the nucleic acid molecule to a Reference to identify a segment of the physical map that has a co-relationship to the at least a segment of the Reference
iv) correlating the segment of the physical map of at least a portion of the nucleic acid molecule that differs from the correlating Reference to a region of interest on the nucleic acid molecule;
v) subjecting the region of interest on the nucleic acid molecule to a second physical characterization.

2. The method of claim 1, wherein the surface is exposed.

3. The method of claim 1, wherein the surface is not interior to a flow cell.

4. The method of claim 1, wherein the surface is not interior to a fluidic device.

5. The method of claim 1, wherein the surface is accessible to exterior mechanical manipulation.

6. The method of claim 1, wherein attaching the nucleic acid molecule comprises binding a chromatin constituent associated with the nucleic acid molecule to a chromatin constituent affinity partner.

7. The method of claim 1, wherein attaching comprises immobilizing the nucleic acid to the surface.

8. The method of claim 1, wherein determining a physical map of at least a portion of the nucleic acid molecule comprises determining an AT concentration of the at least a portion of the nucleic acid molecule.

9. The method of claim 1, wherein determining a physical map of at least a portion of the nucleic acid molecule comprises determining a GC concentration of the at least a portion of the nucleic acid molecule.

10. The method of claim 1, wherein determining a physical map of at least a portion of the nucleic acid molecule comprises determining a nucleic acid subsequence pattern for a recurring subsequence of the at least a portion of the nucleic acid molecule.

11. The method of claim 10, wherein the nucleic acid subsequence pattern comprises a repeat element pattern.

12. The method of claim 11, wherein the repeat element comprises a transposon.

13. The method of claim 11, wherein the repeat element comprises a retroelement.

14. The method of claim 11, wherein the repeat element comprises an Alu repeat.

15. The method of claim 11, wherein the repeat element comprises an octomer.

16. The method of claim 11, wherein the repeat element comprises a hexamer.

17. The method of claim 1, wherein determining a physical map of at least a portion of the nucleic acid molecule comprises determining a nucleic acid higher order structure pattern.

18. The method of claim 17, wherein the nucleic acid higher order structure pattern comprises a nucleic acid knot pattern.

19. The method of claim 17, wherein the nucleic acid higher order structure pattern comprises a nucleic acid binding protein binding pattern.

20. The method of claim 17, wherein the nucleic acid higher order structure pattern comprises a topological pattern.

21. The method of claim 1, wherein determining a physical map of at least a portion of the nucleic acid molecule comprises determining a nucleic acid associate protein binding pattern.

22. The method of claim 21, wherein the nucleic acid associate protein binding pattern is a chromatin protein binding pattern.

23. The method of claim 21, wherein the nucleic acid associate protein binding pattern is an exogenous protein binding pattern.

24. The method of claim 21, wherein the nucleic acid associate protein binding pattern is a CRISPR protein complex binding pattern.

25. The method of claim 21, wherein the nucleic acid associate protein binding pattern is a transcription factor binding pattern.

26. The method of claim 21, wherein the nucleic acid associate protein binding pattern is a histone binding pattern.

27. he method of claim 21, wherein the nucleic acid associate protein binding pattern is a modified histone binding pattern.

28. The method of claim 1, wherein determining a physical map of at least a portion of the nucleic acid molecule comprises determining a nucleic acid modification pattern.

29. The method of claim 28, wherein the nucleic acid modification pattern results from contacting bound labelling bodies.

30. The method of claim 28, wherein the nucleic acid modification pattern is a DNA methylation pattern.

31. The method of claim 1, wherein determining a physical map of at least a portion of the nucleic acid molecule does not comprise sequencing the at least a portion of the nucleic acid molecule.

32. The method of claim 1, wherein determining a physical map of at least a portion of the nucleic acid molecule requires no more than 1 second.

33. The method of claim 1, wherein determining a physical map of at least a portion of the nucleic acid molecule requires no more than 1/100 of a second.

34. The method of claim 1, wherein the comparing comprises aligning.

35. The method of claim 34, wherein aligning the physical map of at least a portion of the nucleic acid molecule to a reference comprises identifying a segment of the physical map of at least a portion of the nucleic acid molecule that is absent from the reference.

36. The method of claim 34, wherein aligning the physical map of at least a portion of the nucleic acid molecule to a Reference comprises identifying a segment of the physical map of at least a portion of the nucleic acid molecule that is inverted relative to the Reference.

37. The method of claim 34, wherein aligning the physical map of at least a portion of the nucleic acid molecule to a Reference comprises identifying a segment of the physical map of at least a portion of the nucleic acid molecule is translocated relative to the Reference.

38. The method of claim 34, wherein aligning the physical map of at least a portion of the nucleic acid molecule to a Reference comprises identifying a segment of the physical map of at least a portion of the nucleic acid molecule that that is duplicated relative to the Reference.

39. The method of claim 34, wherein aligning the physical map of at least a portion of the nucleic acid molecule to a Reference comprises identifying a segment of the physical map of at least a portion of the nucleic acid molecule that differs by at least 5% relative to the Reference.

40. The method of claim 34, wherein aligning the physical map of at least a portion of the nucleic acid molecule to a Reference comprises identifying a segment of the physical map of at least a portion of the nucleic acid molecule that that differs by at least 10% relative to the Reference.

41. The method of claim 34, wherein aligning the physical map of at least a portion of the nucleic acid molecule to a Reference comprises identifying a segment of the physical map of at least a portion of the nucleic acid molecule that differs by at least 20% relative to the Reference.

42. The method of claim 34, wherein aligning the physical map of at least a portion of the nucleic acid molecule to a Reference to identify a segment of the physical map of at least a portion of the nucleic acid molecule that differs by at least 50% relative to the Reference.

43. The method of claim 1, wherein the Reference comprises a predictive physical map.

44. The method of claim 1, wherein the Reference is derived from a nucleic acid sequence.

45. The method of claim 44, wherein the nucleic acid sequence is a genomic sequence.

46. The method of claim 44, wherein the nucleic acid sequence is derived from a reference organism.

47. The method of claim 44, wherein the nucleic acid sequence is derived from a cancer-free cell.

48. The method of claim 1, wherein the Reference is previously obtained.

49. The method of claim 1, wherein the Reference is concurrently obtained.

50. The method of claim 1, wherein the Reference is obtained from a tissue distal to a tissue from which the nucleic acid molecule is obtained.

51. The method of claim 50, wherein the tissue and the nucleic acid are obtained from a common individual.

52. The method of claim 50, wherein the tissue is disease free.

53. The method of claim 50, wherein the tissue is cancer free.

54. The method of claim 50, wherein the nucleic acid molecule is obtained from a cancerous cell.

55. The method of claim 50, wherein the tissue is cancerous.

56. The method of claim 50, wherein the tissue exhibits a disease.

57. The method of claim 50, wherein the nucleic acid molecule is obtained from a healthy cell.

58. The method of claim 50, wherein the nucleic acid molecule is obtained from a disease-free cell.

59. The method of claim 50, wherein the tissue and the nucleic acid differ in age.

60. The method of claim 59, wherein the tissue is a preserved tissue.

61. The method of claim 59, wherein the nucleic acid is from a later obtained cell.

62. The method of claim 59, wherein the nucleic acid is from an earlier obtained cell.

63. The method of claim 1, wherein correlating the segment of the physical map of at least a portion of the nucleic acid molecule that differs from the Reference to a region of interest on the nucleic acid molecule comprises identifying a location of the region of interest on the nucleic acid molecule on the surface.

64. The method of claim 1, wherein subjecting the region of interest on the nucleic acid molecule to a second physical characterization comprises removing a cover slip covering the nucleic acid molecule.

65. The method of claim 1, wherein subjecting the region of interest on the nucleic acid molecule to a second physical characterization occurs on an exposed area of the surface.

66. The method of claim 1, wherein subjecting the region of interest on the nucleic acid molecule to a second physical characterization comprises generating a second physical characterization of the region of interest on the nucleic acid molecule.

67. The method of claim 66, wherein the second physical characterization depicts a characteristic different from that initially characterized.

68. The method of claim 66, wherein the second physical characterization depicts an AT pattern.

69. The method of claim 66, wherein the second physical characterization depicts a purine/pyrimidine pattern.

70. The method of claim 66, wherein the second physical characterization depicts a protein binding pattern.

71. The method of claim 66, wherein the second physical characterization depicts secondary structure concentration.

72. The method of claim 66, wherein the second physical characterization depicts a histone modification pattern.

73. The method of claim 66, wherein the second physical characterization depicts a nucleic acid modification pattern.

74. The method of claim 66, wherein the second physical characterization depicts an octomer distribution pattern.

75. The method of claim 66, wherein the second physical characterization depicts a hexamer distribution pattern.

76. The method of claim 66, wherein the second physical characterization depicts a transposable element pattern.

77. The method of claim 66, wherein the second physical characterization comprises a nucleic acid probe binding pattern.

78. The method of claim 66, wherein the second physical characterization presents the number of repeats of a repeated element.

79. The method of claim 77, wherein the nucleic acid probe binding pattern is assayed using a fluorophore bound to a nucleic acid probe.

80. The method of claim 77, wherein the nucleic acid probe binding pattern is assayed using a barcode tag bound to a nucleic acid probe.

81. The method of claim 66, wherein the second physical characterization comprises obtaining a nucleic acid sequence.

82. The method of claim 66, wherein the second physical characterization comprises subjecting the region to a contact probe.

83. The method of claim 82, wherein the contact probe determines a nucleic acid sequence for at least a portion of the region.

84. The method of claim 82, wherein the contact probe is an atomic force microscopy probe.

85. The method of claim 82, wherein the contact probe determines a position of the region in an axis perpendicular to the region.

86. The method of claim 66, wherein the second physical characterization comprises physically manipulating the region.

87. A method of analyzing a nucleic acid, comprising generating a physical map of the nucleic acid in no more than 1 second, comparing the physical map to a reference, and generating a second physical map of a portion of the nucleic acid.

88. The method of claim 87, wherein the portion of the nucleic acid that differs from the reference is inverted relative to the reference.

89. The method of claim 87, wherein the portion of the nucleic acid that differs from the reference is translocated relative to the reference.

90. The method of claim 87, wherein the portion of the nucleic acid that differs from the reference is duplicated relative to the reference.

91. The method of claim 87, wherein the portion of the nucleic acid that differs from the reference is absent from the reference.

92. The method of claim 87, wherein the second physical map comprises a sequence of the portion of the nucleic acid that differs from the reference.

93. The method of claim 92, wherein the sequence is determined in situ.

94. The method of claim 92, wherein the sequence is determined by direct manipulation of the nucleic acid on the surface.

95. The method of claim 92, wherein the sequence is determined using atomic force microscopy.

96. The method of claim 92, wherein the sequence is determined using hybridization to a probe of known sequence.

97. The method of claim 87, wherein the nucleic acid is fixed to a surface.

98. The method of claim 97, wherein the surface is exposed.

99. The method of claim 97, wherein the surface is not a flow cell interior.

100. The method of claim 97, wherein the surface is accessible to physical manipulation.

101. The method of claim 97, wherein the surface is covered by a removable cover slip.

102. A system for analyzing a nucleic acid comprising an open surface to which the nucleic acid is attached, a lens for capturing an optical signal indicative of a physical map of the nucleic acid, and an contact probe for determining a characteristic of a subregion of the nucleic acid.

103. The system of claim 102, comprising a stored reference physical map and a processing unit to compare the stored reference physical map to a nucleic acid physical map generated from the fluorescence.

104. The system of claim 103, wherein the processing unit is configured to identify a difference between the stored reference physical map to the nucleic acid physical map generated from the optical signal.

105. A method of analyzing a nucleic acid, comprising
a. attaching the nucleic acid to a surface;
b. determining a physical map for at least a portion of the nucleic acid;
c. using the physical map to identify a region of interest in the nucleic acid molecule; and
d. subjecting the region of interest on the nucleic acid molecule to a second physical characterization.

106. The method of claim 105, wherein using the physical map to identify a region of interest comprises comparing the physical map to a reference, and correlating a landmark on the reference to the physical map to identify a region of interest in the nucleic acid molecule.

107. The method of claim 106, wherein the physical map does not differ from the reference.

108. The method of claim 106, wherein the physical map differs from the reference.

109. The method of claim 106, wherein the landmark is a known variable region on the reference.

110. The method of claim 106, wherein the landmark aligns with the region of interest.

111. The method of claim 106, wherein the landmark is removed a known distance from a region on the reference that corresponds to the region of interest on the nucleic acid molecule.

112. The method of claim 106, wherein the second physical characterization comprises a higher resolution map at the region of interest on the nucleic acid molecule than the physical map.

113. The method of claim 106, wherein the second physical characterization comprises a nucleic acid sequence of the region of interest of the nucleic acid.

114. The method of claim 106, wherein the second physical characterization comprises determining a second physical map of the region of interest.

115. The method of claim 105, wherein determining the physical map on the nucleic acid molecule does not preclude subjecting the region of interest on the nucleic acid molecule to a second physical characterization.

116. The method of claim 105, wherein the reference is a physical map of a nucleic acid from a non-diseased cell.

117. The method of claim 105, wherein the reference is a physical map of a nucleic acid from a diseased cell.

118. The method of claim 105, wherein the reference is a physical map of a nucleic acid from a cell exhibiting a phenotype of interest.

119. The method of claim 105, wherein the reference is derived from a nucleic acid sequence.

120. The method of claim 119, wherein the nucleic acid sequence is a genomic nucleic acid sequence.

121. A method of analyzing a population of nucleic acids, comprising generating distinct physical maps of members of the population of nucleic acids, and directing a contact probe to a region within at least one physical map, wherein at least one physical map is generated per molecule within the population per second.

122. The method of claim 121, wherein the physical maps are generated successively.

123. The method of claim 121, wherein the physical maps are generated concurrently.

124. The method of claim 121, comprising generating second physical maps of a portion of at least some of the nucleic acids.

125. The method of claim 122, wherein the second physical maps represent subsets of the distinct physical maps.

126. The method of claim 125, wherein the second physical maps target regions identified through comparison to at least one reference.

127. The method of claim 125, wherein the second physical maps target regions that differ among the distinct physical maps of members of the population of nucleic acids.

128. A method of characterizing a region of interest of a nucleic acid molecule, comprising
a. attaching the nucleic acid molecule to a surface of at least one point on the nucleic acid
b. determining a physical map of at least a portion of the nucleic acid molecule
c. identifying at least one landmark by comparing the physical map of at least a portion of the nucleic acid molecule to a reference
d. calculating the spatial extent of a region of interest relative to the landmark
e. subjecting the region of interest on the nucleic acid molecule to a second physical characterization.

129. The method of claim 128, wherein attaching comprises immobilizing.

130. The method of claim 128, wherein comparing comprises aligning.

131. The method of claim 128, wherein calculating the spatial extent of a region of interest comprises calculating the smallest rectangle inclusive of two or more landmarks.

132. The method of claim 128, wherein calculating the spatial extent of a region of interest comprises calculating the coordinates of an enclosed area containing the landmark whereby the landmark is not closer than 1 um to any point in the periphery.

133. The method of claim 128, wherein calculating the spatial extent of a region of interest comprises calculating the coordinates of an enclosed area that is a fixed distance upstream or downstream of the landmark.

134. The method of claim 128, wherein calculating the spatial extent of a region of interest comprises calculating the coordinates of an enclosed area based on a landmark and scaled by the observed distances between two or more landmarks.

135. The method of claim 128, wherein calculating the spatial extent of a region of interest comprises calculating the coordinates of an enclosed area to be a fixed distance from a landmark and excluding regions devoid of nucleic acids.

136. The method of claim 128, wherein identifying comprises finding regions of the physical map that differ from the Reference.

137. The method of claim 128, wherein identifying comprises finding regions of the physical map that are similar to a specific portion of the Reference.
